(19) United States
(12) Patent Application Publication
(10) Pub. No.: US 2026/0179717 A1
(43) Pub. Date: Jun. 25, 2026

(54) MULTI-SCALE FOOTPRINTING OF DNA-PROTEIN INTERACTIONS

(71) Applicants: President and Fellows of Harvard College, Cambridge, MA (US); The Broad Institute, Inc., Cambridge, MA (US); The Children’s Medical Center Corporation, Boston, MA (US)

(72) Inventors: Jason Daniel Buenrostro, Belmont, MA (US); Maximilian Alexander Horlbeck, Brookline, MA (US); Ruochi Zhang, Cambridge, MA (US); Yan Hu, Cambridge, MA (US)

(73) Assignees: President and Fellows of Harvard College, Cambridge, MA (US); The Broad Institute, Inc., Cambridge, MA (US); The Children’s Medical Center Corporation, Boston, MA (US)

(21) Appl. No.: 19/169,896

(22) Filed: Apr. 3, 2025

Related U.S. Application Data
(60) Provisional application No. 63/737,511, filed on Dec. 20, 2024.

(51) Int. Cl. G16B 5/00; G16B 40/00

(52) U.S. Cl. CPC G16B 5/00; G16B 40/00

(57) ABSTRACT

Multi-scale footprinting of DNA-protein interactions is described. Multi-scale footprint scores may be generated based on chromatin accessibility data, the multi-scale footprint scores indicating protein binding to positions of a genome at different protein size scales. A deep learning model may be trained using the multi-scale footprint scores and corresponding DNA sequences. DNA-protein interactions for a DNA sequence of interest may be predicted using the trained deep learning model. The prediction may include generating sequence attribution scores for the DNA sequence of interest using the trained deep learning model and predicting transcription factor binding sites of the DNA sequence of interest based on the sequence attribution scores.

### RELATED APPLICATION

This application claims priority to U.S. Provisional Patent Application Ser. No. 63/737,511, filed Dec. 20, 2024, entitled “Multi-Scale Footprinting of DNA-Protein Interactions,” the entire disclosure of which is hereby incorporated by reference herein in its entirety.

### STATEMENT REGARDING GOVERNMENT SUPPORT

This invention was made with government support under Grant Nos. HL131477, HL151353, HG011986, GM007748, and HD052896 awarded by the National Institutes of Health. The government has certain rights in the invention.

### BACKGROUND

Chromatin accessibility and the binding of regulatory proteins to DNA are involved in gene regulation and cellular function. Understanding the organization and dynamics of these interactions across different cell types and conditions may help decipher the complexities of gene expression and cellular identity. Traditional methods for studying protein-DNA interactions, such as chromatin immunoprecipitation followed by sequencing (ChIP-seq), have provided valuable insights but are limited in the ability to comprehensively map the binding of all regulatory proteins across diverse cellular contexts.

High-throughput sequencing technologies have enabled methods such as deoxyribonuclease I hypersensitive sites sequencing (DNase-seq) and assay for transposase-accessible chromatin using sequencing (ATAC-seq) to probe genome-wide chromatin accessibility. These techniques have advanced the identification of regulatory regions and enable transcription factor binding to be inferred. However, existing computational approaches for analyzing this data often struggle to accurately detect protein binding events, particularly for factors that do not leave strong footprints or in the context of single-cell experiments.

Furthermore, current methods typically focus on a narrow size range of protein-DNA interactions, primarily centered around transcription factor binding sites. As a result, the positioning of larger protein complexes, such as nucleosomes, may not be observed, and the understanding of the interplay between different classes of DNA-binding proteins on gene regulation may be incomplete.

### SUMMARY

Multi-scale footprinting of DNA-protein interactions is described. Multi-scale footprint scores for may be generated based on chromatin accessibility data, the multi-scale footprint scores indicating protein binding to positions of a genome at different protein size scales. A deep learning model may be trained using the multi-scale footprint scores and corresponding DNA sequences. DNA-protein interactions for a DNA sequence of interest may be predicted using the trained deep learning model. The prediction may include generating sequence attribution scores for the DNA sequence of interest using the trained deep learning model and predicting transcription factor binding sites of the DNA sequence of interest based on the sequence attribution scores.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ multi-scale footprinting of DNA-protein interactions.

FIG. 2 depicts an example overview of generating the DNA-protein interaction predictions.

FIG. 3 illustrates an example training process that may be used in generating the at least one sequence model of the DNA-protein interaction analysis module of FIG. 1.

FIG. 4 depicts an example implementation of using the at least one sequence model to analyze DNA sequences for DNA-protein interaction predictions.

FIG. 5 depicts an example procedure for multi-scale footprinting of DNA-protein interactions as described herein.

FIG. 6 illustrates an example system including various components of an example device that can be implemented as any type of computing device as described and/or utilized with reference to FIGS. 1-5 to implement the techniques described herein.

FIGS. 7A and 7B show an overview of the multi-scale footprinting workflow and a bar plot comparing the performance of different Tn5 bias correction models.

FIG. 8 shows a schematic illustration of the Tn5 bias prediction model.

FIG. 9 depicts predicted Tn5 bias, observed Tn5 insertion, and multi-scale footprints on BAC DNA incubated with 0 nM (upper) or 100 nM (lower) MYC/MAX at example region chr2:238237173-238237972.

FIG. 10 depicts aggregate multi-scale footprints on BAC DNA incubated with 0 nM or 100 nM MYC/MAX at MYC/MAX motif sites for a previous ATAC-footprinting method (TOBIAS) and PRINT.

FIG. 11 depicts box plots showing footprint scores at motif sites or background (non-motif) sites with 0 nM or 100 nM TFs for a previous ATAC-footprinting method (TOBIAS) and PRINT.

FIG. 12 shows multi-scale footprints at two adjacent MYC/MAX motif sites with 0 nM (upper), 50 nM (middle), and 100 (lower) nM MYC/MAX.

FIG. 13 shows multi-scale footprints in the cCRE region of chr6:154732971-154733770 in HepG2.

FIG. 14 shows aggregate multi-scale footprints for example TFs including AR, CREB1, TFE3, and NFIA.

FIG. 15 schematically shows an overview of footprint-to-object prediction and sequence-to-footprint prediction (seq2PRINT) models and their applications.

FIG. 16 shows a schematic illustration of the seq2PRINT model.

FIG. 17 shows observed and predicted multi-scale footprints in the example region chr4:39181940-39182739.

FIG. 18 depicts a bar plot showing median precision of TF binding prediction by different methods.

FIG. 19 shows a schematic illustration of using low-rank adaptation (LoRA) to scale up seq2PRINT training on large numbers of samples (pseudo-bulks).

FIG. 20 shows a percentage of all cCREs with the given complexity of TF binding modes.

FIG. 21 shows a line plot showing TF binding scores of representative TFs within erythroid cCREs across the pseudo-time of erythroid differentiation and a change in the maximum distance between adjacent nucleosomes in erythroid cCREs across differentiation pseudo-time.

FIG. 22 shows an example comparing the timing of TF binding and the relative position of TF binding sites to cCRE centers.

FIG. 23 shows a schematic overview of data generation and analysis of HSC and progenitor cells.

FIG. 24 depicts a bar plot showing differential RNA results for partner TFs forming composite motifs with Ets1.

FIG. 25 shows age-associated nucleosome changes.

FIG. 26 shows TF motif enrichment at nucleosome footprints lost during aging (absolute difference in footprint score >1 and FDR<0.01).

FIG. 27 shows a schematic of age-associated cCRE reorganization.

FIG. 28 shows Tn5 bias and observed Tn5 insertion for a genomic region that is footprinted.

### DETAILED DESCRIPTION

### Overview

Cis-regulatory elements (CREs) are regions of non-coding deoxyribonucleic acid (DNA) involved in controlling gene expression and cellular function. These DNA sequences, which may include enhancers, promoters, and other regulatory regions, can serve as binding sites for various proteins, such as transcription factors and chromatin modifiers. CREs may be located proximal to or distal from the genes they regulate, and their activity can be influenced by chromatin accessibility and the binding of regulatory proteins. The term “cis” indicates these elements are located on the same DNA molecule as the gene they regulate.

CREs may exhibit dynamic behavior, with their structure and function changing in response to cellular processes, environmental stimuli, or developmental cues. The organization and composition of proteins bound to CREs can vary across different cell types and conditions, potentially contributing to the specificity and plasticity of gene regulation.

Understanding the complex interplay between CREs, their associated proteins, and the resulting effects on gene expression may be valuable for deciphering the mechanisms underlying cellular identity, differentiation, and disease states. However, comprehensively mapping and characterizing CREs across diverse cellular contexts has remained challenging with traditional experimental approaches.

There is a need for improved approaches that can leverage chromatin accessibility data to provide a more comprehensive and accurate picture of protein-DNA interactions across multiple scales. Such methods could enable deeper insights into the dynamic reorganization of regulatory elements during cellular processes like differentiation and aging, which may reveal new mechanisms of gene regulation and cellular function.

To overcome these issues, multi-scale footprinting of DNA-protein interactions is described herein. In accordance with the described techniques, multi-scale footprint scores for a genome of a sample are generated based on chromatin accessibility data, such as ATAC-seq data. These multi-scale footprint scores indicate protein binding to positions of the genome at different protein size scales, such as size scales ranging from 4 base pairs to 200 base pairs. As used herein, a “footprint” represents a region of DNA that is protected from cleavage or degradation due to the binding of a transcription factor or other DNA-binding protein, resulting in reduced accessibility to enzymes like transposases used in ATAC-seq experiments. A “footprint score” refers to a quantitative measure of protein binding at a specific genomic position. For example, a higher footprint score indicates a greater likelihood of protein occupancy at that position. In at least one implementation, a multi-scale footprint may be visualized as a matrix or heatmap, corresponding to footprint scores at a specific genomic position (e.g., x-axis) and size scale (e.g., y-axis). The multi-scale nature of this analysis allows for the simultaneous detection and characterization of DNA-binding proteins of diverse sizes, from small transcription factors to larger complexes like nucleosomes. Accordingly, a comprehensive view of protein-DNA interactions across the genome may be provided.

The footprint scores, along with corresponding DNA sequences, may be used to train a deep learning model. Through training, the deep learning model, which may be implemented as a convolutional neural network (CNN), “learns” to predict protein binding patterns from DNA sequence information. The trained deep learning model may generate sequence attribution scores for DNA sequences, which can be further analyzed to predict transcription factor binding sites and identify de novo motifs. A “sequence attribution score” refers to a numerical value assigned to individual nucleotides or short sequences within a genomic region that quantifies the contribution of those nucleotides or short sequences to protein binding, for example, as observed in the multi-scale footprint. By way of example, a higher attribution score indicates a DNA sequence that has a greater influence on protein binding, as inferred by the trained deep learning model.

In one or more implementations, the trained deep learning model may be used to generate additional fine-tuned models for specific cell states or conditions using, for example, low rank adaptation (LoRA). By way of example, an initial model may be trained using aggregated data from multiple cell states. Subsequently, a fine-tuned model may be generated from the initial model using data specific to particular cell type(s) and/or condition(s). The fine-tuned parameters may be represented as a low-rank decomposition, allowing for computationally efficient adaptation of the initial model to diverse cellular contexts compared with separately training multiple models.

By leveraging chromatin accessibility data and deep learning approaches, the techniques described herein enable the genome-wide prediction of protein binding events without individual ChIP-seq experiments for each transcription factor, thus reducing experimental resources and cost and increasing the amount of information that can be determined from the chromatin accessibility data. The multi-scale nature of the footprinting allows for the detection of various protein-DNA interactions of different size scales, from small transcription factors to larger complexes like nucleosomes, which may provide insights into the organization and dynamics of regulatory elements. Furthermore, the use of deep learning models allows for the discovery of novel DNA binding motifs and the capture of complex sequence patterns that may not be apparent through conventional motif analysis. As a result, insights into gene regulation, cellular differentiation, and disease mechanisms at high genomic and cell-state resolution may be obtained.

The techniques described herein provide a technology-based solution for studying protein-DNA interactions by enabling comprehensive, genome-wide predictions of DNA-protein interactions and regulatory element identification at multiple spatial scales in a manner that is not possible with manual analysis by a human. By way of example, the trained deep learning model may identify patterns and correlations in the data that would be impractical or impossible for a human to discern manually, especially given the vast quantities of data produced in chromatin accessibility experiments. Furthermore, the ability to fine-tune models for specific cell states using low-rank adaptation allows for efficient adaptation to diverse cellular contexts, which would be prohibitively time-consuming and complex, if not impossible, via manual analysis. The integration of de novo motif discovery with deep learning predictions enables the identification of novel regulatory elements and transcription factor binding sites without relying on existing databases, which overcomes the limitations of traditional manual curation approaches. Overall, these computational techniques enable rapid, scalable, and precise analyses of gene regulation mechanisms across entire genomes and multiple cell types and/or conditions, providing insights that would not be possible to obtain through manual human data analysis.

In some aspects, the techniques described herein relate to a method for predicting DNA-protein interactions, including: receiving chromatin accessibility data; generating multi-scale footprint scores based on the chromatin accessibility data, the multi-scale footprint scores indicating protein binding to positions of a genome at different protein size scales; training a deep learning model using the multi-scale footprint scores and corresponding DNA sequences; and predicting the DNA-protein interactions for a DNA sequence of interest using the trained deep learning model, the predicting including: generating sequence attribution scores for the DNA sequence of interest using the trained deep learning model; and predicting transcription factor binding sites of the DNA sequence of interest based on the sequence attribution scores.

In some aspects, the techniques described herein relate to a method, wherein the chromatin accessibility data includes an assay for transposase-accessible chromatin using sequencing (ATAC-seq) data, and wherein generating the multi-scale footprint scores includes: defining a center footprint region and two flanking regions for a position of the genome; calculating a ratio of transposase insertions in the center footprint region to total insertions in both the center footprint region and the two flanking regions; and determining a footprint score for the position of the genome by comparing the calculated ratio to a background distribution defining an expected distribution of transposon insertion ratios when no protein is bound at the position of the genome.

In some aspects, the techniques described herein relate to a method, wherein the multi-scale footprint scores are generated for window sizes ranging from 4 base pairs to 200 base pairs.

In some aspects, the techniques described herein relate to a method, wherein training the deep learning model includes: encoding DNA sequences of the genome into one-hot encoded matrices; generating, by the deep learning model, a predicted multi-scale footprint based on the one-hot encoded matrices; and adjusting parameters of the deep learning model based on the predicted multi-scale footprint to minimize a difference with a corresponding multi-scale footprint score generated from the chromatin accessibility data.

In some aspects, the techniques described herein relate to a method, wherein the deep learning model includes a convolutional neural network, and adjusting the parameters of the deep learning model includes adjusting weights and biases of the convolutional neural network.

In some aspects, the techniques described herein relate to a method, further including: identifying de novo motifs for the transcription factor binding sites based on the sequence attribution scores by clustering and aligning regions of high sequence attribution scores.

In some aspects, the techniques described herein relate to a method, wherein predicting the transcription factor binding sites of the DNA sequence of interest based on the sequence attribution scores includes: generating transcription factor binding scores for regions of the DNA sequence of interest using a transcription factor binding prediction model; and indicating whether a given portion of the DNA sequence of interest is predicted to bind a transcription factor based on a corresponding transcription factor binding score.

In some aspects, the techniques described herein relate to a method, further including: training the transcription factor binding prediction model using the sequence attribution scores and chromatin immunoprecipitation followed by sequencing (ChIP-seq) data for a training sample, wherein the ChIP-seq data provides ground truth labels for bound transcription factors with respect to the sequence attribution scores.

In some aspects, the techniques described herein relate to a method, wherein the chromatin accessibility data includes single-cell ATAC-seq data, and the method further includes: defining cell states of the single-cell ATAC-seq data by clustering single cells; and generating a low-rank adaptation (LoRA) model for a specific cell state by: fine-tuning a subset of parameters of the trained deep learning model using the single-cell ATAC-seq data corresponding to the specific cell state; and representing the fine-tuned subset of parameters as a low-rank decomposition.

In some aspects, the techniques described herein relate to a method, further including: generating multiple LoRA models for a plurality of different cell states represented in the single-cell ATAC-seq data; and analyzing DNA-protein interactions across diverse cellular contexts by comparing outputs of the multiple LoRA models.

In some aspects, the techniques described herein relate to a system for predicting DNA-protein interactions, including: a DNA-protein interaction analysis module implemented as instructions stored in a non-transitory computer-readable storage medium that, when executed by a processor, cause the processor to perform operations including: generating, using a deep learning model trained with multi-scale footprint data indicating protein binding to positions of a genome at different protein size scales, sequence attribution scores for a DNA sequence of interest; predicting transcription factor binding sites of the DNA sequence of interest based on the sequence attribution scores; and predicting de novo motifs for the transcription factor binding sites based on the sequence attribution scores.

In some aspects, the techniques described herein relate to a system, wherein the multi-scale footprint data are generated from transposase-accessible chromatin using sequencing (ATAC-seq) data, and wherein generating the multi-scale footprint data includes: defining, via a footprinting model of the DNA-protein interaction analysis module, a center footprint region and two flanking regions for a position of the genome; calculating, via the footprinting model of the DNA-protein interaction analysis module, a ratio of transposase insertions in the center footprint region to total insertions in both the center footprint region and the two flanking regions; and outputting, by the footprinting model of the DNA-protein interaction analysis module, a footprint score for the position of the genome by comparing the calculated ratio to a background distribution defining an expected distribution of transposon insertion ratios when no protein is bound at the position of the genome.

In some aspects, the techniques described herein relate to a system, wherein the operations further include: training the deep learning model by: encoding DNA sequences of the genome into one-hot encoded matrices; generating, by the deep learning model, a predicted multi-scale footprint based on the one-hot encoded matrices; and adjusting parameters of the deep learning model based on the predicted multi-scale footprint compared to a corresponding multi-scale footprint of the multi-scale footprint data, the multi-scale footprint data generated from chromatin accessibility data.

In some aspects, the techniques described herein relate to a system, wherein predicting the de novo motifs includes: clustering, via a de novo motif discovery algorithm of the DNA-protein interaction analysis module, regions of the DNA sequence of interest with high sequence attribution scores; aligning, via the de novo motif discovery algorithm, the clustered regions; and outputting, by the de novo motif discovery algorithm, recurring sequence patterns corresponding to previously uncharacterized DNA binding sites for proteins based on the aligned clustered regions.

In some aspects, the techniques described herein relate to a system, wherein the operations further include: generating a low-rank adaptation (LoRA) model for a specific cell state from the deep learning model by: fine-tuning a subset of parameters of the trained deep learning model using single-cell ATAC-seq data corresponding to the specific cell state; and representing the fine-tuned subset of parameters as a low-rank decomposition; and predicting DNA-protein interactions for the DNA sequence of interest using the LoRA model.

In some aspects, the techniques described herein relate to a method for analyzing DNA-protein interactions in single-cell data, including: training a deep learning model using multi-scale footprint scores and corresponding DNA sequences, the multi-scale footprint scores derived from bulk chromatin accessibility data corresponding to multiple cell states; defining cell states by clustering single cells; generating a plurality of low-rank adaptation (LoRA) models, each of the plurality of LoRA models corresponding to a specific cell state, by fine-tuning a subset of parameters of the trained deep learning model using single-cell chromatin accessibility data corresponding to the specific cell state for a given model of the plurality of LoRA models; and predicting cell state-specific DNA-protein interactions for a target DNA sequence via the plurality of LoRA models.

In some aspects, the techniques described herein relate to a method, wherein the fine-tuned subset of parameters are represented as a low-rank decomposition.

In some aspects, the techniques described herein relate to a method, further including: training the deep learning model by: encoding DNA sequences into one-hot encoded matrices; generating, by the deep learning model, a predicted multi-scale footprint based on the one-hot encoded matrices; and adjusting parameters of the deep learning model based on the predicted multi-scale footprint compared to a corresponding multi-scale footprint score generated from the bulk chromatin accessibility data.

In some aspects, the techniques described herein relate to a method, wherein predicting the cell state-specific DNA-protein interactions for the target DNA sequence via the plurality of LoRA models includes: generating sequence attribution scores for the target DNA sequence using a LoRA model corresponding to a cell state of interest; and predicting transcription factor binding sites of the target DNA sequence based on the sequence attribution scores.

In some aspects, the techniques described herein relate to a method, further including: identifying cell state-specific regulatory elements by comparing DNA-protein interactions predicted by different LoRA models of the plurality of LoRA models; and tracking changes in transcription factor binding patterns across a cellular differentiation trajectory.

In the following discussion, an example environment is first described that may employ the techniques described herein. Example implementation details and procedures are then described which may be performed in the example environment as well as other environments. Consequently, performance of the example procedures is not limited to the example environment and the example environment is not limited to performance of the example procedures.

### Example Environment

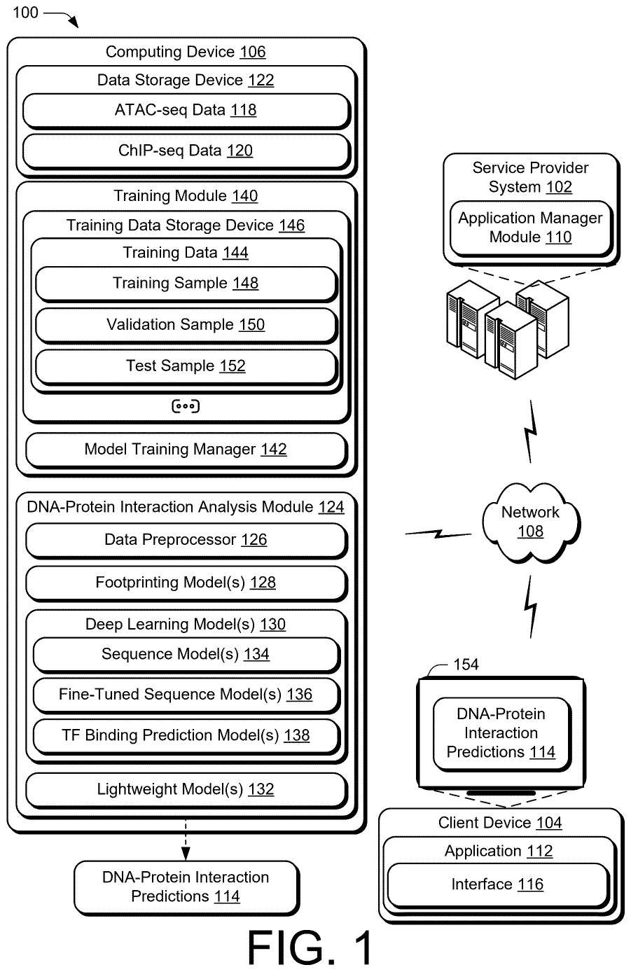

FIG. 1 is an illustration of an environment 100 in an example implementation that is operable to employ multi-scale footprinting of DNA-protein interactions. The illustrated environment 100 includes a service provider system 102, a client device 104, and a computing device 106 that are communicatively coupled, one to another, via a network 108. The network 108 may enable wired and/or wireless electronic communication, for example. Although the computing device 106 is illustrated as separate from the service provider system 102 and the client device 104, this functionality may be incorporated as part of the service provider system 102 and/or the client device 104, further divided among other entities, and so forth. By way of example, an entirety of or portions of the functionality of the computing device 106 may be incorporated as part of the service provider system 102 and/or the client device 104. Additionally or alternatively, an entirety of or portions of the client device 104 may be incorporated as part of the service provider system 102 and/or the computing device 106.

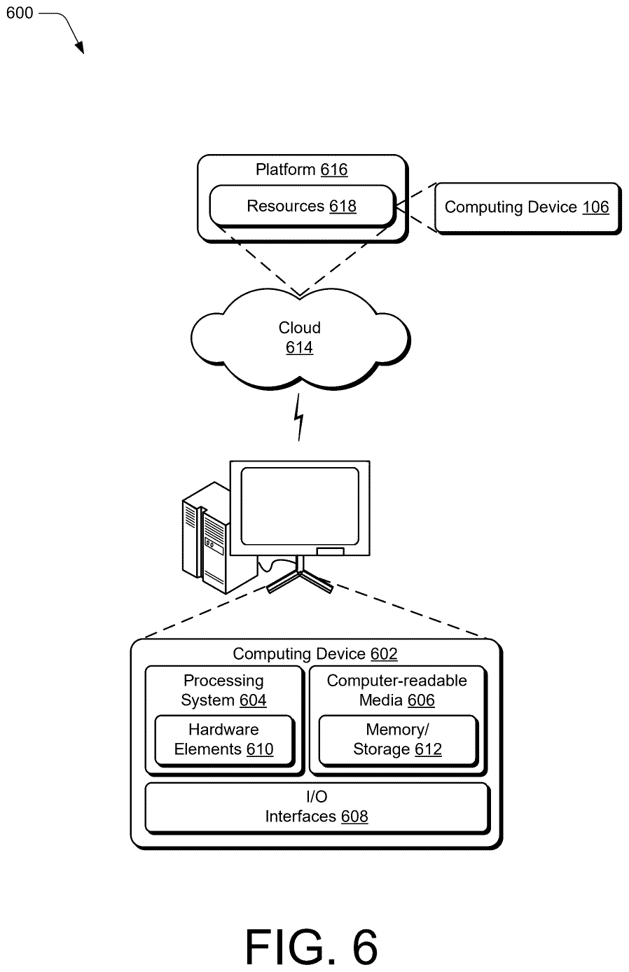

Computing devices that are usable to implement the service provider system 102, the client device 104, and the computing device 106 may be configured in a variety of ways. A computing device, for instance, may be configured as a desktop computer, a laptop computer, a mobile device (e.g., assuming a handheld configuration such as a tablet or mobile phone), and so forth. Thus, the computing device may range from full resource devices with substantial memory and processor resources (e.g., personal computers) to a low-resource device with limited memory and/or processing resources (e.g., mobile devices). Additionally, a computing device may be representative of a plurality of different devices, such as multiple servers utilized to perform operations “over the cloud,” as further described in relation to FIG. 6.

The service provider system 102 is illustrated as including an application manager module 110 that is representative of functionality to provide access to the computing device 106 to a user of the client device 104 via the network 108. The application manager module 110, for instance, may expose content or functionality of the computing device 106 that is accessible via the network 108 by an application 112 of the client device 104. The application 112 may be configured as a network-enabled application, a browser, a native application, and so on, that exchanges data with the service provider system 102 via the network 108. The data can be employed by the application 112 to enable the user of the client device 104 to communicate with the service provider system 102, such as to receive application updates and features when the service provider system 102 provides functionality to manage the application 112.

In the context of the described techniques, the application 112 includes functionality to train and/or use machine learning models to analyze chromatin accessibility data and output DNA-protein interaction predictions 114, as will be elaborated herein. By way of example, the DNA-protein interaction predictions 114 may include footprint scores indicating the likelihood of protein binding at specific genomic locations. Additionally, the predictions may provide information on transcription factor binding sites, nucleosome positioning, and other DNA-protein interactions. In some cases, the DNA-protein interaction predictions 114 may also include an indication of binding strength or occupancy. The predictions may further incorporate specific DNA sequence features or patterns that contributed to the interaction assessment.

In the illustrated example, the application 112 includes an interface 116 that is implemented at least partially in hardware of the client device 104 for facilitating communication between the client device 104 and the computing device 106. By way of example, the interface 116 includes functionality to receive inputs to the computing device 106 from the client device 104 (e.g., from a user of the client device 104) and output information, data, and so forth from the computing device 106 to the client device 104, including the DNA-protein interaction predictions 114.

The computing device 106 illustrated in FIG. 1 is further configured to receive an assay for transposase-accessible chromatin using sequencing (ATAC-seq) data 118 and chromatin immunoprecipitation followed by sequencing (ChIP-seq) data 120, which are stored in a data storage device 122. The data storage device 122 may represent one or more databases and other types of storage capable of storing the ATAC-seq data 118 and ChIP-seq data 120. The data storage device 122 may also store a variety of other data, such as genomic annotations, cell type information, and so forth. The ATAC-seq data 118 may comprise individual genomic regions as well as genome-wide accessibility profiles. The ChIP-seq data 120 may comprise protein binding locations across the genome. In at least one implementation, the ATAC-seq data 118 and ChIP-seq data 120 are stored in a manner to facilitate retrieval thereof according to its order or time of acquisition.

The data storage device 122 may comprise any known data storage medium. It is to be appreciated that while the data storage device 122 is illustrated as part of the computing device 106, in at least one variation, the data storage device 122 is part of another computing device, such as an external storage component or data server. By way of example, the components of the computing device 106 may be coupled to one another to form a single structure, may be separate but located within a common room, or may be remotely located with respect to one another. For example, one or more of the modules described herein may operate in a data server that has a distinct and remote location with respect to other components of the computing device 106. Optionally, the computing device 106 may be a unitary system that is capable of being moved (e.g., portably) from room to room. For example, the computing device 106 may be transported (e.g., on a cart) or comprise a desktop or laptop device.

In at least one implementation, the ATAC-seq data 118 and/or the ChIP-seq data 120, or a portion thereof, may be processed by a DNA-protein interaction analysis module 124. By way of example, the DNA-protein interaction analysis module 124 is representative of the functionality implemented at least partially in hardware of the computing device 106 to analyze the ATAC-seq data 118 and ChIP-seq data 120, such as one or more sequencing datasets, and output the DNA-protein interaction predictions 114. In the example shown in FIG. 1, the DNA-protein interaction analysis module 124 includes a data preprocessor 126, at least one footprinting model 128, at least one deep learning model 130, at least one lightweight model 132 for analyzing the input data to generate the DNA-protein interaction predictions 114. The at least one deep learning model 130, for instance, may further include at least one sequence model 134, at least one fine-tuned sequence model 136, and at least one transcription factor (TF) binding prediction model 138. The at least one deep learning model 130, for instance, is a trained machine learning model. By way of example, the DNA-protein interaction analysis module 124 may include multiple different deep learning models that correspond to different types of machine learning models, where the underlying models learn using different approaches (e.g., supervised learning, unsupervised learning, and/or reinforcement learning), and/or multiple different deep learning models having a same model architecture but that are trained using different input data and/or to output different types of DNA-protein interaction predictions 114. By way of example, these models may include regression models (e.g., linear, polynomial, and/or logistic regression models), classifiers, transformer networks, neural networks (e.g., convolutional neural networks, recurrent neural networks), state space models, and reinforcement learning based models, to name just a few.

The at least one deep learning model 130 may be configured as (or include) other types of models without departing from the spirit or scope of the described techniques. These different machine learning models may be built or trained (or the model otherwise learned), respectively, using different inputs and/or different algorithms due, at least in part, to different architectures and/or learning paradigms. Accordingly, it is to be appreciated that the following discussion of the functionality of the DNA-protein interaction analysis module 124 is applicable to a variety of machine learning models. For explanatory purposes, however, the functionality of the at least one deep learning model 130 will be described generally with respect to a convolutional neural network (CNN). The CNN, for instance, may include a 1D CNN architecture to process DNA sequences input as one-hot encoded matrices. Additional details of the CNN will be described herein. In one or more implementations, the CNN is combined with additional architectures and/or model portions to produce the DNA-protein interaction predictions 114.

In one or more implementations, the at least one fine-tuned sequence model 136 may be derived from the at least one sequence model 134 using low rank adaptation (LoRA). In LoRA, an initial deep learning model (e.g., the at least one sequence model 134) is first trained using the ATAC-seq data 118 aggregated from multiple cell states. Subsequently, a subset of parameters of this initial model is fine-tuned using the ATAC-seq data 118 specific to a particular cell state or condition. By way of example, cell states of single-cell ATAC-seq data may be defined by clustering single cells. The fine-tuned parameters may be represented as a low-rank decomposition. This approach may reduce the number of trainable parameters compared to fine-tuning the entire at least one sequence model 134, which may be more computationally efficient for adapting the at least one sequence model 134 to specific cellular contexts. The LoRA technique may be applied to generate multiple fine-tuned models for different cell types or conditions, which may enable the analysis of DNA-protein interactions across diverse cellular contexts. In at least one implementation, the LoRA fine-tuning process may use single-cell ATAC-seq data 118 aggregated into pseudo-bulks representing specific cell states or conditions. This may allow the at least one fine-tuned sequence model 136 to capture cell type-specific or condition-specific protein-DNA interaction patterns.

The computing device 106 further includes a training module 140 that is implemented at least partially in hardware of the computing device, at least in part, to deploy deep learning to generate the at least one deep learning model 130. By way of example, the training module 140 includes a model training manager 142 that is configured to manage the deep learning model 130. This model management may include, for example, building the deep learning model 130, training the deep learning model 130, updating the model(s), and so forth. For instance, the model training manager 142 may be configured to carry out this model management using, at least in part, training data 144 maintained in a training data storage device 146. For example, the model training manager 142 may use at least a portion of the training data 144 as input for training the at least one deep learning model 130. The training data 144 may include ATAC-seq and ChIP-seq data from one or more data sources, for example. As such, the test sample 152 may be used to verify that the deep learning model 130 achieves performance goals. Ellipses denote that more than one training data set may be stored in the training data storage device 146.

It is to be appreciated that although the training data 144 is shown in a single training data storage device 146, in at least one variation, the training data 144 may be distributed among multiple storage locations. Alternatively, or in addition, the training data storage device 146 may be stored in a location that is external to the computing device 106 and accessed by the computing device 106 (e.g., over the network 108). As such, it is to be appreciated that the relative arrangement of the various modules and data storage devices in FIG. 1 is non-limiting, and variations are possible.

In one or more implementations, the training data 144 is further subdivided into a training sample 148, a validation sample 150, and a test sample 152. By way of example, the training sample 148 may comprise a largest portion of the training data 144, while the validation sample 150 and/or the test sample 152 may comprise a smallest portion of the training data 144. As a non-limiting example, the training sample 148 comprises 80% of the training data 144, the validation sample 150 comprises 10% of the training data 144, and the test sample 152 comprises 10% of the training data 144, although other divisions are possible. The training sample 148, for instance, may comprise between 50% and 80% of the training data 144, the validation sample 150 may comprise between 10% and 40% of the training data 144, and the test sample 152 may comprise between 5% and 30% of the training data 144.

Broadly speaking, the training sample 148 may be input to the deep learning model 130 during a training process, where the at least one deep learning model 130 learns patterns and relationships in the data. During the training process, weights and parameters of the deep learning model 130 may be adjusted to reduce (e.g., minimize) errors between an output of the model and a ground truth label associated with corresponding ATAC-seq and ChIP-seq data (e.g., the DNA-protein interaction predictions, as determined by experimental validation). Following completion of the training process, the at least one deep learning model 130 is able to accurately predict the DNA-protein interaction predictions 114 of the training sample 148.

The validation sample 150 may be input to the at least one deep learning model 130 during a model refinement process, where the at least one deep learning model 130 is adjusted to prevent or reduce overfitting/underfitting of the model to the training sample 148. By way of example, the model refinement process may be performed following each round (or epoch) of training to evaluate how well the deep learning model 130 performs on data that is different from the training sample 148. During the model refinement process, for instance, a complexity, learning rate, and/or regularization of the at least one deep learning model 130 may be adjusted (e.g., by the model training manager 142, automatically and/or based on user input) based on the performance of the at least one deep learning model 130 with the validation sample 150. As an illustrative example, if the deep learning model 130 accurately predicts the DNA-protein interaction predictions 114 of the training sample 148 but not the validation sample 150, overfitting of the at least one deep learning model 130 to the training sample 148 is indicated. As such, the model refinement process enables settings of the at least one deep learning model 130 and/or its training to be fine-tuned so that the deep learning model 130 can be generalized to unseen data (e.g., data that the deep learning model 130 has not been trained on).

The test sample 152 may be input to the deep learning model 130 during an internal validation process that is performed after the at least one deep learning model 130 is trained and fine-tuned. The test sample 152 comprises data that was unseen by the deep learning model 130 during the training and model refinement processes described above but that is derived from the same dataset. The internal validation process evaluates the performance of the at least one deep learning model 130 on similar data as to that used during the training and model refinement processes. If the at least one deep learning model 130 does not meet acceptable or desired performance criteria (e.g., as defined by model developers) during the internal validation process, the at least one deep learning model 130 may be returned to the training and/or model refinement processes so that changes can be made. For example, changes may be made to feature selection, the model architecture, regularization techniques, hyperparameter tuning, and the like.

The model training manager 142 may leverage the functionality of the data preprocessor 126 to process the training data 144 during the training, refinement, and validation processes described above. The data preprocessor 126, for instance, may remove sample identifying information and standardize the ATAC-seq and ChIP-seq data input into the at least one deep learning model 130 such as by normalizing, upsampling, and/or padding the input chromatin accessibility and protein binding data.

In response to the deep learning model 130 meeting desired or acceptable performance metrics, the at least one deep learning model 130 may be deployed for determining the DNA-protein interaction predictions 114 of newly obtained ATAC-seq and ChIP-seq data, including data for which there are no ground truth labels. By way of example, the ATAC-seq data 118 and ChIP-seq data 120 may correspond to data that have not been experimentally validated with respect to the DNA-protein interaction predictions 114. The ATAC-seq data 118 and/or the ChIP-seq data 120 may be input into the (trained and validated) at least one deep learning model 130 at or around the time of acquisition, and the at least one deep learning model 130 may output the DNA-protein interaction predictions 114 accordingly, thus enabling a streamlined chromatin accessibility analysis workflow.

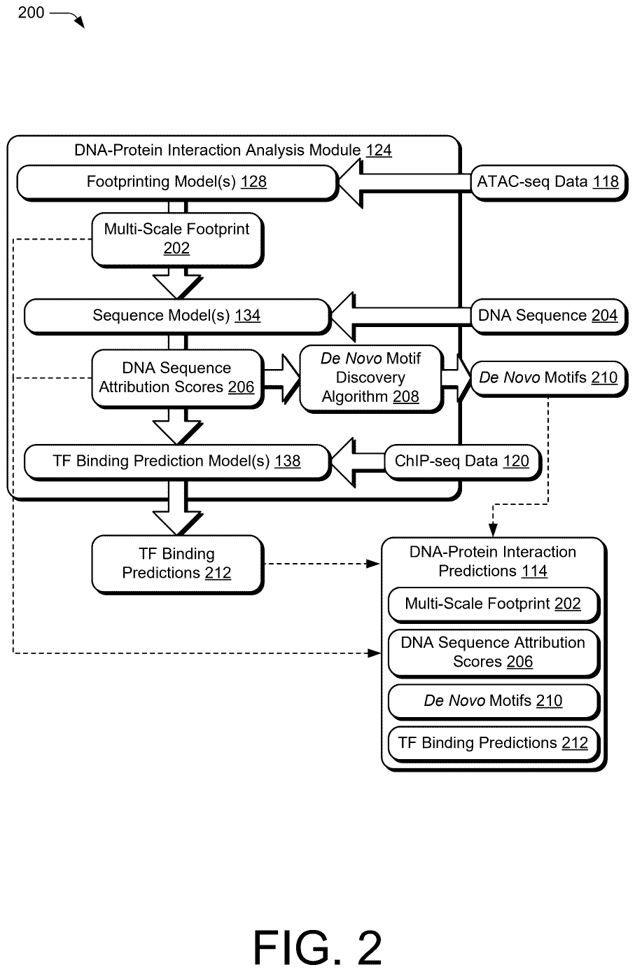

In at least one implementation, the DNA-protein interaction predictions 114 include potential interactions between DNA and various proteins within candidate cis-regulatory elements (cCREs). As will be further elaborated herein, e.g., with respect to FIG. 2, the DNA-protein interaction predictions 114 may include multi-scale footprints, which are patterns of DNA protection from enzymatic cleavage indicative of protein binding at various spatial scales. This may further include sequence-based predictions of these footprints, where the DNA sequence is used to predict the multi-scale footprint patterns that would be observed experimentally. Additionally, or alternatively, the DNA-protein interaction predictions 114 may include predictions of transcription factor binding and/or de novo motifs for protein binding sequences. The DNA-protein interaction predictions 114 may provide a comprehensive characterization of the protein-binding landscape within cCREs. Such predictions may offer insights into gene regulation and chromatin structure at high genomic and cell-state resolution, which may enable the identification and functional annotation of regulatory elements across diverse cellular contexts.

The client device 104 is shown displaying, via a display device 154, the DNA-protein interaction predictions 114. Alternatively, or in addition, the client device 104 may display, via the display device 154, the ATAC-seq data 118 or ChIP-seq data 120. It is to be appreciated that the DNA-protein interaction predictions 114 may be also stored in a memory of the computing device 106 and/or the client device 104 for subsequent access.

In this way, the DNA-protein interaction analysis module 124 enables automated analysis of chromatin accessibility data for identifying and/or predicting DNA-protein interactions, which may be used in studying gene regulation, cellular differentiation, and disease mechanisms.

### Multi-Scale Footprinting of DNA-Protein Interactions

FIG. 2 depicts an example overview 200 of generating the DNA-protein interaction predictions. The overview 200 shows one implementation of the DNA-protein interaction analysis module 124 of FIG. 1 in greater detail.

The at least one footprinting model 128 receives the ATAC-seq data 118 as an input. The ATAC-seq data 118 is processed by the at least one footprinting model 128 to generate a multi-scale footprint 202. The multi-scale footprint 202 represents protein-DNA interactions derived from chromatin accessibility data across multiple spatial scales. By way of example, the multi-scale footprint 202 may include footprint scores calculated for a range of window sizes, typically from 4 base pairs to 200 base pairs, centered on a given genomic position. These scores indicate the likelihood and strength of protein binding at that position for proteins of various sizes, from smaller transcription factors to larger complexes like nucleosomes. As such, a footprint score refers to a quantitative measure of protein binding at a specific genomic position. The footprint score for a given genomic position may be calculated by the at least one footprinting model 128 by comparing an observed ratio of transposase insertions in a center region to flanking regions against an expected background distribution. A higher footprint score indicates a greater likelihood of protein occupancy at that position. The multi-scale nature of this representation allows for the simultaneous detection and characterization of DNA-binding proteins of diverse sizes.

The multi-scale footprint 202 is provided as an input to the at least one sequence model 134, which also receives a DNA sequence 204, which may be any DNA sequence of interest. By way of example, the sequence of interest may be a specific promoter region associated with a gene under investigation, an enhancer region, or a coding sequence. Intergenic regions may be examined in some implementations. In at least one implementation, the DNA sequence 204 may correspond to an entire chromosome or whole genome. As yet another example, the DNA sequence 204 may correspond to a specific locus associated with a disease, condition, or other phenotype. The DNA sequence 204 may be a newly sequenced genomic region from an uncharacterized organism. Additionally, or alternatively, the DNA sequence 204 may be a synthetic and/or engineered DNA sequence. By way of example, a known DNA sequence may be perturbed in silico, and the resulting modified DNA sequence may be evaluated to investigate the effects of the perturbation on DNA-protein interactions. The at least one sequence model 134 may thus enable analysis of various genomic contexts, which may provide insights into transcriptional regulation mechanisms, long-range gene regulation, novel regulatory elements, protein binding patterns across large genomic scales, genetic factors in health and disease, regulatory functions maintained across species, regulatory elements in novel genetic contexts, and so forth.

The at least one sequence model 134 generates DNA sequence attribution scores 206. The DNA sequence attribution scores 206 include numerical values assigned to individual nucleotides or short sequences within a genomic region that quantify the contribution of those nucleotides or short sequences to the multi-scale footprint 202. Higher DNA sequence attribution scores 206 indicate DNA sequences that have a greater influence on protein binding, as inferred by the at least one sequence model 134.

It is to be appreciated that in at least one variation, the at least one fine-tuned sequence model 136 is used in addition to or as an alternative to the at least one sequence model 134. By way of example, the at least one fine-tuned sequence model 136 may be tailored to a specific cell type or state and may be derived from the at least one sequence model 134. The at least one fine-tuned sequence model 136 may improve prediction accuracy for the particular biological context being analyzed and/or may capture cell type-specific or condition-specific DNA-protein interaction patterns in the DNA sequence attribution scores 206.

In the example of the overview 200, the DNA sequence attribution scores 206 are processed by a de novo motif discovery algorithm 208 to identify de novo motifs 210. The de novo motifs 210 represent previously uncharacterized or novel DNA binding sites for proteins (e.g., transcription factors). By way of example, the de novo motifs 210 are computationally determined rather than relying on pre-existing databases of known protein binding sequences. In at least one implementation, the de novo motif discovery algorithm 208 includes functionality for analyzing regions having high values for the sequence attribution scores 206. The de novo motif discovery algorithm 208 may cluster and align these high-scoring regions to identify recurring sequence patterns and may further apply statistical methods to evaluate the significance of these patterns compared to background genomic sequences. The de novo motif discovery algorithm 208 may filter and refine the identified patterns to produce the de novo motifs 210. The DNA-protein interaction analysis module 124 thus enables the discovery of novel DNA binding motifs without prior knowledge of protein-DNA interactions, which may reveal new regulatory elements or transcription factor binding sites that were not previously characterized in existing databases.

The DNA sequence attribution scores 206 are also input to the at least one TF binding prediction model 138, which outputs TF binding predictions 212. The TF binding predictions 212 are computational estimates of where transcription factors are likely to bind within the DNA sequence 204. By way of example, the TF binding predictions 212 may provide a probabilistic assessment of transcription factor occupancy at specific genomic locations, allowing for genome-wide mapping of potential regulatory interactions without the need for individual ChIP-seq experiments for each TF. In at least one implementation, the de novo motifs 210 may be further utilized by the at least one TF binding prediction model 138 to improve the accuracy of the TF binding predictions 212.

In one or more implementations, the at least one TF binding prediction model 138 also receives the ChIP-seq data 120, which may provide training data for the at least one TF binding prediction model 138. By way of example, the TF binding predictions 212 output by the at least one TF binding prediction model 138 may be compared to the ChIP-seq data 120, which may serve as experimentally validated ground truth labels for the TF binding predictions 212.

One or more or each of the multi-scale footprint 202, the DNA sequence attribution scores 206, the de novo motifs 210, and the TF binding predictions 212 are output as the DNA-protein interaction predictions 114. As such, the DNA-protein interaction analysis module 124 allows for a flexible analysis of the DNA-protein interaction predictions 114.

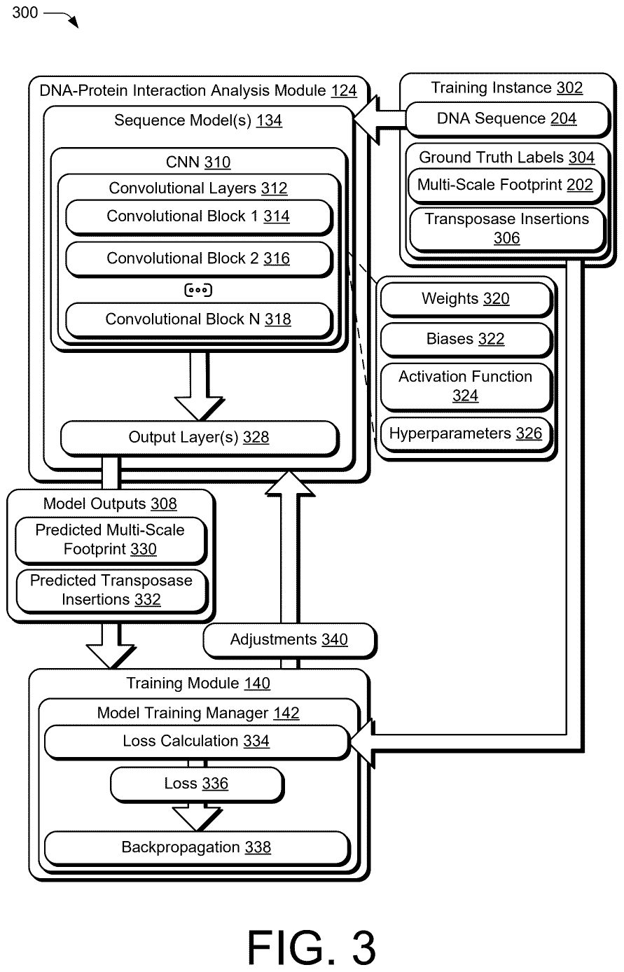

FIG. 3 illustrates an example training process 300 that may be used in generating the at least one sequence model 134 of the DNA-protein interaction analysis module 124 of FIG. 1. It is to be appreciated that the example training process 300 denotes one implementation of a training process that may be used in generating at least a portion of the at least one sequence model 134.

In the example training process 300, a training instance 302 includes a DNA sequence 204 and ground truth labels 304 associated with the DNA sequence 204. The DNA sequence 204, for instance, includes a one-hot encoded DNA sequence obtained from genomic data. In at least one implementation, the DNA sequence 204 comprises a sequence of length L encoded into an L×4 matrix, where each row has one element set to 1 representing the specific nucleotide. During the training, the DNA sequence 204 is part of a corresponding portion of the training data 144 (e.g., the training sample 148).

Each DNA sequence 204 may be separately evaluated by the deep learning model 134 to generate model outputs 308, which correspond to the single DNA sequence 204. As such, the training instance 302 includes an input portion (e.g., the DNA sequence 204) and an associated expected output portion (e.g., the ground truth labels 304), and a great many training instances 302 may be used during the training process. The model outputs 308 correspond to predictions for multi-scale footprints at base-pair resolution. The ground truth labels 304 define true or expected outputs of the at least one sequence model 134 for the DNA sequence 204. In the training process 300, the ground truth labels 304 include the multi-scale footprint 202 (e.g., as generated via the at least one footprinting model 128) and transposase insertions 306. By way of example, the transposase insertions 306 may be observed transposase (e.g., Tn5) insertion sites from ATAC-seq data and may thus provide information about chromatin accessibility. The multi-scale footprint 202 represents the protein binding patterns at different spatial scales, as derived from the chromatin accessibility data, for instance.

In the example training process 300 shown in FIG. 3, the DNA sequence 204 is provided to the at least one sequence model 134. The at least one sequence model 134 comprises a convolutional neural network (CNN) 310 having convolutional layers 312. The convolutional layers 312 are shown as including a first convolutional block 314 (e.g., “convolutional block 1”), a second convolutional block 316 (e.g., “convolutional block 2”), and an N^{th} convolutional block 318 (e.g., “convolutional block N”), where N is an integer representing the total number of convolutional blocks. Ellipses denote that additional convolutional blocks may be present between the second convolutional block 316 and the N^{th} convolutional block 318. As a non-limiting example, there may be eight convolutional blocks. In general, the convolutional layers 312 may be configured to extract features of the DNA sequence 204.

The convolutional layers 312 may comprise weights 320, a bias 322, an activation function 324, and hyperparameters 326. By way of example, the weights 320 and the bias 322 may be randomly initialized and then “learned” during the training process. The CNN 310, for instance, performs a series of convolutions. A convolution is a mathematical operation where a kernel (e.g., filter) slides over an input DNA sequence and performs element-wise multiplication with the values of the sequence at each position. The results are summed up to produce a single output value for that position, and this process is repeated across the DNA sequence to produce a feature map.

The first convolutional layer of the convolutional layers 312, for example, may include 1,024 filters of width 21 bp as the weights 320 with Gaussian Error Linear Units (GELUs) activation as the activation function 324, which may capture informative sequence patterns from the DNA sequence 204 (i.e., sequence motifs). The output may be subsequently passed to eight layers of convolutional blocks with residual connection. Each convolutional block may include one grouped dilated convolutional layer (n_filters=1,024, width=3, groups=8, dilation=2\hat{ }i, i=1, . . . , 8) followed by a position-wise feed forward layer (implemented as a convolutional layer with n_filters=1,024, width=1). Batch normalization layers with GELU activations may be inserted between these convolutional blocks.

The hyperparameters 326 are not learned during the training process but can be adjusted to increase performance. The hyperparameters 326 may comprise depth, stride, and zero-padding. For example, the increased dilation rate may result in an expanding receptive field for the CNN 310, capturing the relationship of the sequence patterns and their context.

In at least one implementation, the CNN 310 outputs a feature map, which may be a multi-dimensional representation of the features extracted from the DNA sequence 204. The feature map may capture various characteristics of the DNA sequence at different levels of abstraction. This feature map is then processed through one or more output layers 328 to produce the model outputs 308. The one or more output layers 328 may include fully connected layers, pooling layers, and/or additional convolutional layers, depending on the specific architecture of the at least one sequence model 134.

In at least one implementation, the at one or more output layers 328 includes two output layers. A first output layer generates a predicted multi-scale footprint 330, and a second output layer generates predicted transposase insertions 332. The predicted multi-scale footprint 330 may be a matrix of size L times the number of scales (e.g., multiple footprints may be calculated at different size scales). The predicted transposase insertions 332 may be scalar values for each region of the DNA sequence 204, for instance. The model outputs 308 are received by the model training manager 142, which may perform a loss calculation 334. The loss calculation 334 may use a loss function to compute the difference between the model outputs 308 and the ground truth labels 304, e.g., a loss 336. Various types of loss functions may be used depending on the specific task and model architecture.

In at least one implementation, the loss calculation 334 may be a sum of losses for the model outputs 308. That is, a first loss (e.g., “Loss_1”) may be calculated by comparing the predicted multi-scale footprint 330 and the multi-scale footprint 202, and a second loss (e.g., “Loss_2”) may be calculated by comparing the predicted transposase insertions 332 and the transposase insertions 306 such that the total loss is Loss=Loss_1+Loss_2. In one of more implementations, the first loss and/or the second loss may utilize a mean-squared error loss function. This composite loss function may allow the at least one sequence model 134 to simultaneously learn multiple aspects of DNA-protein interactions.

A goal of the training is to minimize the loss 336 by adjusting the weights 320 and biases 322 of the at least one sequence model 134. In order to do so, the model training manager 142 may employ backpropagation 338 to compute how the parameters are to be updated based on a gradient of the loss 336 with respect to each parameter. The backpropagation 338 results in adjustments 340, which are used to update the weights 320 and biases 322 of the at least one sequence model 134.

As such, following many rounds of training with a large number of training instances 302, the model outputs 308 become consistent with the ground truth labels 304 due to the at least one sequence model 134 “learning” to minimize the loss 336 between the model outputs 308 and the ground truth labels 304.

In this way, the training process 300 provides a comprehensive framework for developing and refining the at least one sequence model 134 for DNA-protein interaction predictions. This approach may enable efficient learning from large datasets of genomic sequences, which may improve the accuracy and generalizability of the DNA-protein interaction predictions.

It is to be appreciated that the at least one TF binding prediction model 138 may be trained similarly in one or more implementations. By way of example, the ChIP-seq data 120 may provide the ground truth labels 304 for a given training instance 302. The ChIP-seq data 120 may indicate bound transcription factors with respect to the DNA sequence attribution scores 206, which may provide the input of the training instance 302.

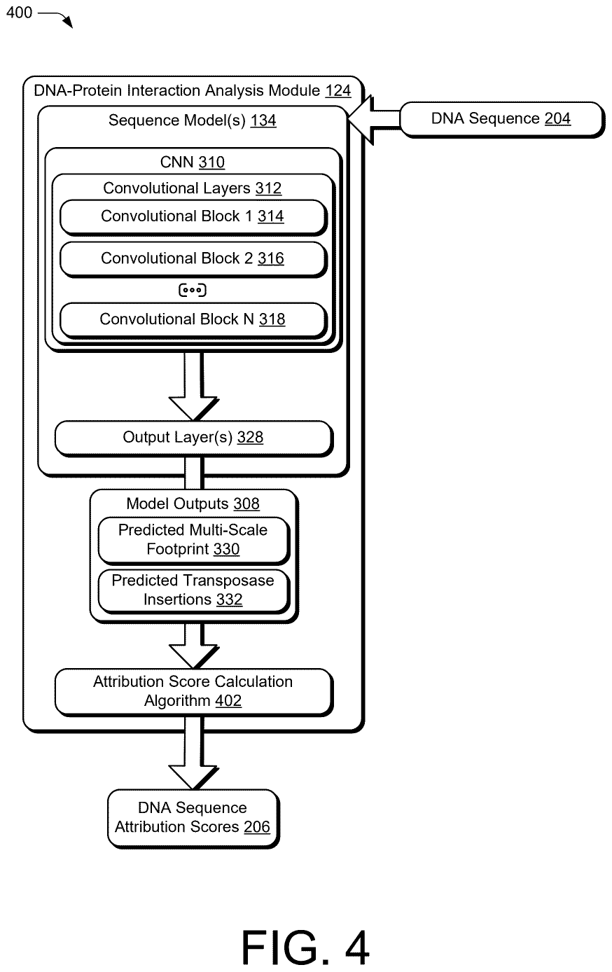

FIG. 4 depicts an example implementation 400 of using the at least one sequence model 134 to analyze DNA sequences for DNA-protein interaction predictions. Components previously introduced in FIGS. 1-3 are numbered the same and function as previously described. It is to be appreciated that the at least one fine-tuned sequence model 136 may operate similarity to the at least one sequence model 134.

The implementation 400 includes processing the DNA sequence 204, which may be a newly acquired DNA sequence data for which there may be no ground truth labels, via the at least one sequence model 134 after the at least one sequence model 134 has been trained.

The model outputs 308 are processed through an attribution score calculation algorithm 402, which analyzes the model outputs 308 to determine the contribution of different DNA sequence features to the predicted protein binding patterns. The attribution score calculation algorithm 402, for instance, may use techniques such as DeepLIFT to calculate attribution scores for each base pair in the DNA sequence 204 based on the predicted multi-scale footprint 330 and/or the predicted transposase insertions 332. The attribution score calculation algorithm 402 may sum footprint scores across entire peak regions for genome-wide calculations, resulting in the DNA sequence attribution scores 206. As such, in at least some implementations, the at least one sequence model 134 is combined with the attribution score calculation algorithm 402 after the training process 300 to output the DNA sequence attribution scores 206.

Having discussed example details of the techniques for the multi-scale footprinting of DNA-protein interactions, consider now an example procedure to illustrate additional aspects of the techniques.

### Example Procedure

This section describes an example procedure for multi-scale footprinting of DNA-protein interactions in one or more implementations. Aspects of the procedure may be implemented in hardware, firmware, or software, or a combination thereof. The procedure is shown as a set of blocks that specify operations performed by one or more devices and are not necessarily limited to the orders shown for performing the operations by the respective blocks. In at least some implementations, at least a portion of the procedure is performed by a suitably configured device, such as the computing device 106 of FIG. 1, by executing instructions stored in a non-transitory computer-readable storage medium.

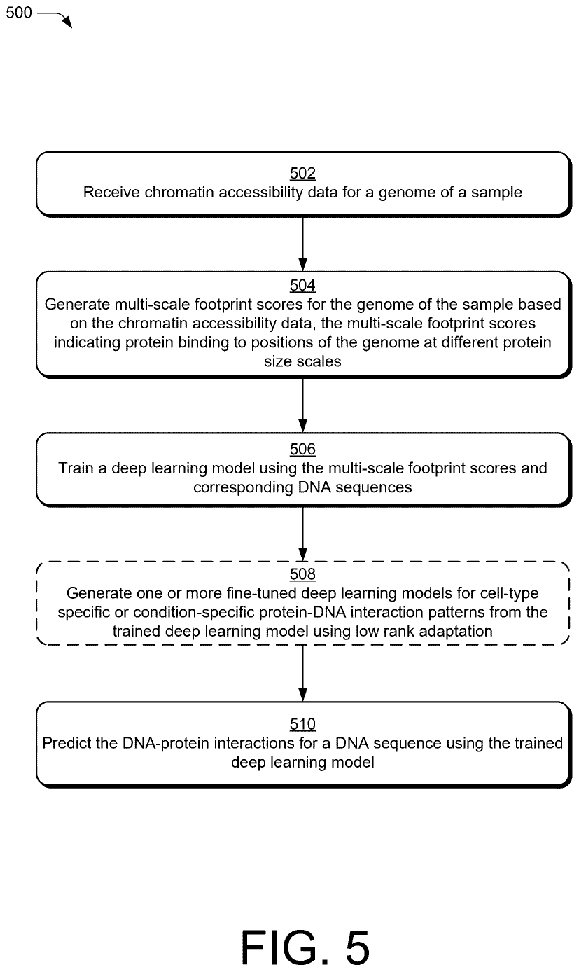

FIG. 5 depicts an example procedure 500 for multi-scale footprinting of DNA-protein interactions as described herein.

Chromatin accessibility data for a genome of a sample is received (block 502). By way of example, the chromatin accessibility data may comprise ATAC-seq data 118, which includes information on regions of open chromatin across the genome. The ATAC-seq data 118 may be stored in the data storage device 122, for instance. The ATAC-seq data 118 reveals areas of the genome that are accessible to regulatory proteins, indicating potential sites of protein-DNA interactions. In at least one implementation, the data preprocessor 126 may apply normalization techniques to account for potential biases in the ATAC-seq data 118. This preprocessing may involve correcting for sequence-specific transposase bias using computational models.

Multi-scale footprint scores are generated for the genome of the sample based on the chromatin accessibility data (block 504). By way of example, the multi-scale footprint scores (e.g., the multi-scale footprint 202 of FIG. 2) indicate protein binding to positions of the genome at different protein size scales. For instance, the at least one footprinting model 128 processes the ATAC-seq data 118 to generate the multi-scale footprint 202, which represents protein binding patterns across various spatial scales.

The at least one footprinting model 128, for instance, may define center footprint regions and flanking regions for positions in the genome. The at least one footprinting model 128 may then calculate ratios of transposase insertions in the center footprint regions to total insertions in the center and flanking regions. These ratios may be compared to background distributions to determine footprint scores for the genomic positions. In at least one implementation, the multi-scale footprint scores are generated for window sizes ranging from 4 base pairs to 200 base pairs in order to detect binding events for various protein sizes, from small transcription factors to larger complexes such as nucleosomes. The resulting multi-scale footprint 202 may comprise a matrix of size L times the number of scales, where L represents the length of the analyzed genomic region. Each element in this matrix may correspond to a footprint score at a specific genomic position and scale.

A deep learning model is trained using the multi-scale footprint scores and corresponding DNA sequences (block 506). By way of example, the deep learning model may be the at least one sequence model 134 and/or the at least one TF binding prediction model 138. As a non-limiting example, the at least one sequence model 134 is trained using the DNA sequence 204 as an input and may output the predicted multi-scale footprint 330 and the predicted transposase insertions 332. The training process may include adjusting weights 320 and biases 322 to minimize the loss 336 between the predicted multi-scale footprint 330 and the biases 322 and the ground truth labels 304. As another example, the at least one TF binding prediction model 138 may be trained using the ChIP-seq data 120 of the same cell line(s) as used to train the at least one sequence model 134. The at least one sequence model 134 may be trained using the ATAC-seq data 118 aggregated from multiple cell states, for instance.

In some implementations, the at least one TF binding prediction model 138 may be trained using the DNA sequence attribution scores 206 along with the ChIP-seq data 120. The ChIP-seq data 120 may provide ground truth labels for bound transcription factors, allowing the at least one TF binding prediction model 138 to “learn” the relationship between sequence features and actual transcription factor binding events.

Optionally, one or more fine-tuned deep learning models for cell-type specific or condition-specific protein-DNA interaction patterns are generated from the trained deep learning model using low rank adaptation (block 508). By way of example, a subset of parameters of the at least one sequence model 134 may be fine-tuned using the ATAC-seq data 118 specific to a particular cell state or condition. The fine-tuned parameters may be represented as a low-rank decomposition. This approach, referred to as low rank adaptation or LoRA, may reduce the number of trainable parameters compared to fine-tuning the entire at least one sequence model 134. As a result, the fine-tuning may be more computationally efficient for adapting the at least one sequence model 134 to specific cellular contexts.

In at least one implementation, the LoRA technique is applied to generate multiple fine-tuned models for different cell types or conditions, which may enable the analysis of DNA-protein interactions across diverse cellular contexts. In at least one implementation, the LoRA fine-tuning process may use single-cell ATAC-seq data 118 aggregated into pseudo-bulks representing specific cell states or conditions. This may allow the at least one fine-tuned sequence model 136 to capture cell type-specific or condition-specific protein-DNA interaction patterns.

DNA-protein interactions are predicted for a target genomic region using the trained deep learning model (block 510). By way of example, after training, the at least one sequence model 134 and/or the at least one fine-tuned sequence model 136 may be combined with the attribution score calculation algorithm 402 to generate DNA sequence attribution scores 206. For example, the attribution score calculation algorithm 402 may receive the model outputs 308 of the at least one sequence model 134 and covert these to the DNA sequence attribution scores 206. In one or more implementations, the DNA sequence attribution scores 206 may be further used to identify de novo motifs 210 via the de novo motif discovery algorithm 208 and/or to produce the TF binding predictions 212 via the at least one TF binding prediction model 138. One or more of these outputs may be combined to form the DNA-protein interaction predictions 114.

By way of example, the de novo motif discovery algorithm 208 may analyze regions of the DNA sequence with high attribution scores. These high-scoring regions are likely to be involved in protein binding and may contain recurring sequence patterns that represent novel binding motifs. The de novo motif discovery algorithm 208 may cluster and align these high-scoring regions to identify common sequence patterns. The de novo motif discovery algorithm 208 may apply statistical methods to evaluate the significance of these patterns compared to background genomic sequences. This approach may enable previously uncharacterized DNA binding motifs to be discovered without relying on existing databases of known protein binding sequences.

The identified de novo motifs 210 may represent binding sites for known transcription factors that were not previously well-characterized, or they may indicate the presence of novel regulatory proteins. The de novo motifs 210 may provide insights into the sequence preferences of DNA-binding proteins and may help identify new regulatory elements within the genome. The discovery of de novo motifs 210 may be particularly valuable for understanding gene regulation in less-studied cell types or organisms where comprehensive databases of binding motifs may not be available.

In some implementations, the de novo motifs 210 may be further validated by comparing them to known motif databases or by experimental techniques such as ChIP experiments (e.g., the ChIP-seq data 120). The integration of de novo motif discovery algorithm 208 with the deep learning-based prediction of the DNA sequence attribution scores 206 may provide a powerful approach for comprehensively mapping the regulatory landscape of the genome.

The at least one TF binding prediction model 138 may generate, as the TF binding predictions 212, transcription factor binding scores for specific regions of the DNA sequence. These scores may indicate the probability or strength of transcription factor binding at each position. The TF binding predictions 212 may include information about which transcription factors are likely to bind, where they are predicted to bind, and with what affinity. The TF binding predictions 212 generated by the at least one TF binding prediction model 138 may be used to annotate regulatory regions, identify potential binding sites for specific transcription factors, and/or predict the effects of sequence variations on transcription factor binding. These predictions may be advantageous for understanding gene regulation in contexts where experimental ChIP-seq data is not available or is difficult to obtain.

In at least one implementation, predicting the DNA-protein interactions may further include identifying cell state-specific regulatory elements by comparing DNA-protein interactions predicted by different LoRA models. For example, the DNA-protein interaction analysis module may analyze and compare the outputs of multiple LoRA models (e.g., the at least one fine-tuned sequence model 136), each corresponding to a different cell state or condition. By examining the differences in predicted protein binding patterns across these models, the DNA-protein interaction analysis module 124 may identify regulatory elements that are active or repressed in specific cell states. By way of example, changes in transcription factor binding patterns may be tracked across a cellular differentiation trajectory, such as when the multiple LoRA models are generated using chromatin accessibility data collected at different time points or stages during cellular differentiation. The DNA-protein interaction predictions 114 may thus indicate how the predicted binding patterns of various transcription factors evolve over time, which may provide insights into the dynamic regulatory processes that govern cell fate decisions and lineage commitment.

In this way, DNA-protein interactions may be predicted across the genome using multi-scale footprinting and deep learning approaches, providing insights into gene regulation and chromatin structure at high genomic and cell-state resolution.

Having described example procedures in accordance with one or more implementations, consider now an example system and device that can be utilized to implement the various techniques described herein.

### Example System and Device

FIG. 6 illustrates an example system generally at 600 that includes an example computing device 602 that is representative of one or more computing systems and/or devices that may implement the various techniques described herein. This is illustrated through inclusion of the computing device 106. The computing device 602 may be, for example, a server of a service provider, a device associated with a client (e.g., a client device), an on-chip system, and/or any other suitable computing device or computing system.

The example computing device 602 as illustrated includes a processing system 604, one or more computer-readable media 606, and one or more I/O interfaces 608 that are communicatively coupled, one to another. Although not shown, the computing device 602 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 604 is representative of functionality to perform one or more operations using hardware. Accordingly, the processing system 604 is illustrated as including hardware elements 610 that may be configured as processors, functional blocks, and so forth. This may include implementation in hardware as an application specific integrated circuit or other logic device formed using one or more semiconductors. The hardware elements 610 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable storage media 606 is illustrated as including memory/storage 612. The memory/storage 612 represents memory/storage capacity associated with one or more computer-readable media. The memory/storage 612 may include volatile media (such as random-access memory (RAM)) and/or nonvolatile media (such as read only memory (ROM), Flash memory, optical disks, magnetic disks, and so forth). The memory/storage 612 may include fixed media (e.g., RAM, ROM, a fixed hard drive, and so on) as well as removable media (e.g., flash memory, a removable hard drive, an optical disc, and so forth). The computer-readable media 606 may be configured in a variety of other ways as further described below.

Input/output interface(s) 608 are representative of functionality to allow a user to enter commands and information to computing device 602, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 602 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hard-wired device that performs operations based on hard-wired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 602. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media, and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 602, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 610 and computer-readable media 606 are representative of modules, programmable device logic and/or fixed device logic implemented in a hardware form that may be employed in some examples to implement at least some aspects of the techniques described herein, such as to cause the hardware to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 610. The computing device 602 may be configured to implement particular instructions and/or functions corresponding to the software and/or hardware modules. Accordingly, implementation of a module that is executable by the computing device 602 as software may be achieved at least partially in hardware, e.g., through use of computer-readable storage media and/or hardware elements 610 of the processing system 604. The instructions and/or functions may be executable/operable by one or more articles of manufacture (for example, one or more computing devices 602 and/or processing systems 604) to implement techniques, modules, and examples described herein.

The techniques described herein may be supported by various configurations of the computing device 602 and are not limited to the specific examples of the techniques described herein. This functionality may also be implemented all or in part through use of a distributed system, such as over a “cloud” 614 via a platform 616 as described below.

The cloud 614 includes and/or is representative of a platform 616 for resources 618, which are depicted including the computing device 106. The platform 616 abstracts underlying functionality of hardware (e.g., servers) and software resources of the cloud 614. The resources 618 may include applications and/or data that can be utilized while computer processing is executed on servers that are remote from the computing device 602. Resources 618 can also include services provided over the Internet and/or through a subscriber network, such as a cellular or Wi-Fi network.

The platform 616 may abstract resources and functions to connect the computing device 602 with other computing devices. The platform 616 may also serve to abstract scaling of resources to provide a corresponding level of scale to encountered demand for the resources 618 that are implemented via the platform 616. Accordingly, in an interconnected device example, implementation of functionality described herein may be distributed throughout the system 600. For example, the functionality may be implemented in part on the computing device 602 as well as via the platform 616 that abstracts the functionality of the cloud 614.

Having discussed example details of the techniques for multi-scale footprinting of DNA-protein interactions, consider now an example to illustrate usage of the techniques.

### Example Application: Multi-Scale Footprints Reveal the Organization of Cis-Regulatory Elements

Cis-regulatory elements (CREs) control gene expression and are dynamic in their structure and function, reflecting changes to the composition of diverse effector proteins over time. However, methods for measuring the organization of effector proteins at CREs across the genome are limited, hampering efforts to connect CRE structure to their function in cell fate and disease. PRINT, a computational method, was developed to identify footprints of DNA-protein interactions from bulk and single-cell chromatin accessibility data across multiple scales of protein sizes. Using these multi-scale footprints, the seq2PRINT framework is described herein, which employs deep learning to allow precise inference of transcription factor and nucleosome binding and interprets regulatory logic at CREs. Applying seq2PRINT to single-cell chromatin accessibility data from human bone marrow revealed sequential establishment and widening of CREs centered on pioneer factors across hematopoiesis. Age-associated alterations in the structure of CREs in murine hematopoietic stem cells were discovered, including widespread reduction of nucleosome footprints and gain of de novo-identified Ets composite motifs. Collectively, a method was established for obtaining rich insights into DNA-binding protein dynamics from chromatin accessibility data and revealing the architecture of regulatory elements across differentiation and aging.

Through homeostasis, development, and disease, cells utilize cis-regulatory elements to regulate gene expression. CREs integrate the binding of structurally diverse regulatory proteins that dynamically move to recruit or evict cooperating factors and determine the overall function and potential of cells. One major challenge in functional genomics is to identify the precise genomic locations and dynamics of these regulatory proteins across all cell types in order to understand the logic of genetic networks and decipher the function of non-coding genetic variation. This presents a challenge in complexity and scale; in humans, cells use diverse combinations of ˜2,000 transcription factors (TFs) to modulate the activity of ˜1 million candidate cis-regulatory elements (cCREs) to regulate the expression of ˜30,000 genes. To decode this regulatory complexity, thousands of ChIP-seq experiments have been performed across a broad range of regulatory proteins and cellular contexts. However, ChIP-based methods cannot scale to measure the binding of all regulatory proteins across all cellular contexts.

Single-cell ATAC-seq (scATAC-seq) has emerged as a powerful and scalable tool for measuring the accessibility of cCREs across the full cellular diversity of fetal and adult tissues. As TFs predominantly bind open chromatin, the intersection of TF motifs with accessible regions is often used as a proxy for TF binding. To achieve higher precision, statistical methods use chromatin accessibility to “footprint” protein binding at cCREs by quantifying the protection of DNA from DNase, MNase, or Tn5 cleavage. However, footprinting methods are limited by the sequence bias of enzymes, focus primarily on TF-scale objects (˜20 bp), do not detect a large fraction of TFs, and/or are not well-adapted to single-cell methods. Recent advances in deep learning have been valuable for investigating diverse aspects of gene regulation, allowing for de novo interpretation and in silico manipulation of the sequence features underlying complex patterns in biological data. Motivated by these advances, the precision of DNA footprinting and the inferential power of deep learning were combined via the approach described herein to generate accurate maps of diverse regulatory proteins from scATAC-seq data at high genomic and cell-state resolution.

A two-step decoding of cCREs is described. First, a tool referred to herein as PRINT (Protein-Regulatory element Interactions at Nucleotide resolution using Transposition) was created that corrects for enzymatic sequence bias and defines multi-scale footprint representations of cCREs, revealing regulatory proteins (e.g., TFs and nucleosomes) of diverse sizes. Then, a deep learning framework that parses the sequence-level organization of multi-scale footprints in cCREs, referred to herein as seq2PRINT, was developed. The seq2PRINT framework enables computationally tractable and precise TF binding prediction in bulk and single-cell ATAC-seq. The seq2PRINT framework was applied to single-cell ATAC-seq and RNA-seq analysis of human bone marrow cells, and TF and nucleosome binding dynamics were tracked across human hematopoiesis. Many cCREs exhibit switching of regulatory TFs through differentiation in a manner not reflected by overall accessibility. Tracking regulatory changes through differentiation elucidated a stepwise model of activation of erythroid and lymphoid cCREs. As epigenetic alterations, including aberrant nucleosome remodeling, are a hallmark of aging, cCRE changes across aging in mouse hematopoietic stem cells (HSCs) were examined. Global alteration of nucleosome positioning within cCREs and age-associated TFs across cCREs were identified. These include both decreased activity of nucleosome-associated TFs, such as Yy1 and Nrf1, and increased binding at de novo motifs representing Ets- and Runx-family members in a broad range of co-binding configurations. Together, these results show that combining multi-scale footprinting with deep learning sequence models is a powerful method for predicting TF binding and elucidating the structural dynamics of cCREs at the genome scale.

### Identification of Multi-Scale Footprints

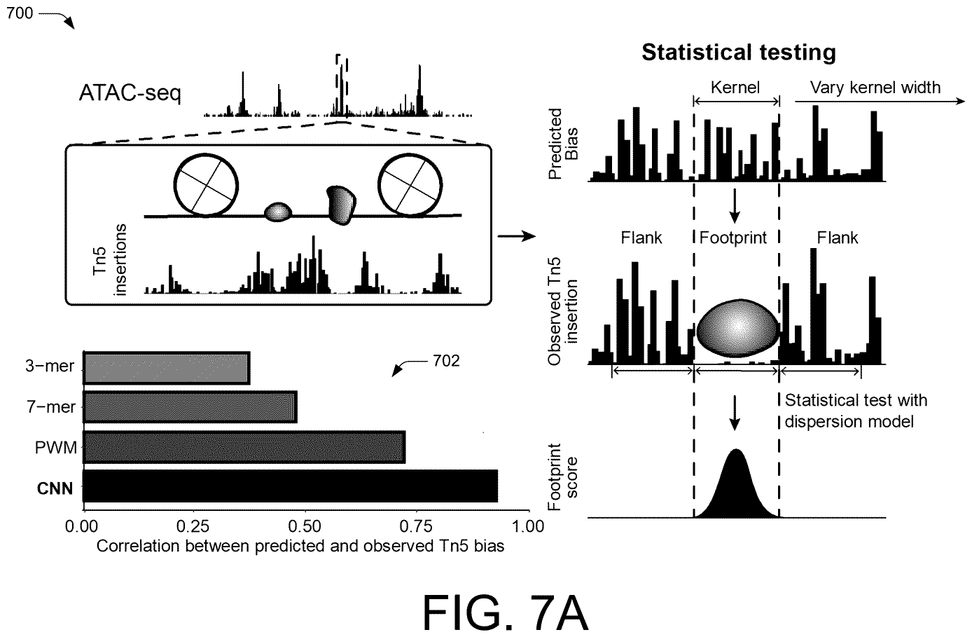

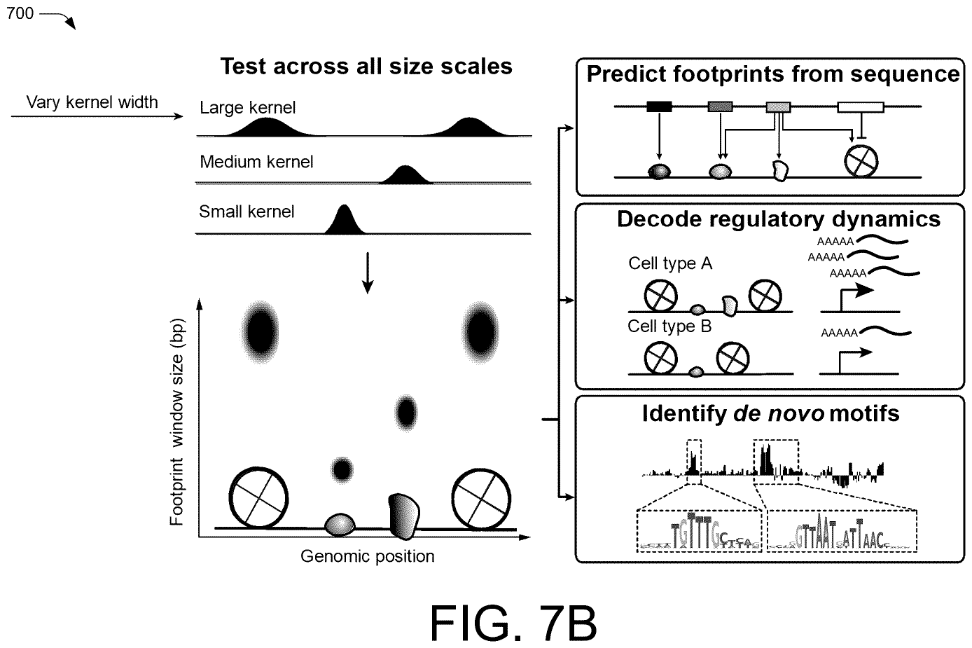

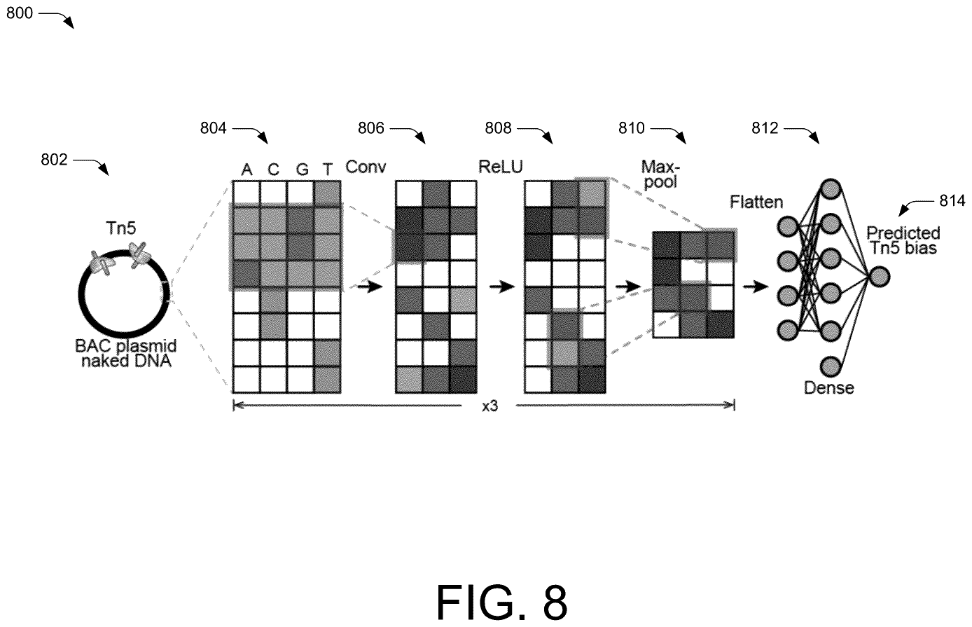

PRINT, a computational approach to detect footprints of DNA-binding proteins of diverse sizes from bulk or single-cell ATAC-seq data (FIGS. 7A and 7B), was developed. To first overcome the sequence bias of Tn5 transposase, which can confound footprint detection, a convolutional neural network (CNN) was trained on Tn5 insertion data on deproteinized DNA from bacterial artificial chromosomes (BACs) (FIG. 8). This model significantly outperformed k-mer and position weight matrix (PWM) models (R=0.94; FIG. 7A), particularly in regions of high GC-content, performed similarly well on Tn5 insertion data from extracted human genomic DNA data (R=0.92), and outperformed Tn5 bias correction by ChromBPNet (FIG. 8).

FIGS. 7A and 7B show an overview of the multi-scale footprinting workflow 700 and a bar plot 702 comparing the performance of different Tn5 bias correction models. Referring first to FIG. 7A, the multi-scale footprinting workflow 700 begins with input ATAC-seq data, which is processed through statistical testing incorporating both a kernel and varying kernel width parameters. The ATAC-seq data is shown with Tn5 insertions displayed as vertical peaks. A schematic representation shows DNA with nucleosomes (e.g., depicted as circular objects) positioned, interspersed with smaller objects representing other DNA-binding proteins. The ATAC-seq data undergoes statistical testing. Both predicted and observed Tn5 insertion patterns are divided into three regions: flank, footprint, and flank. A gray oval shape overlaid on the footprint region in the observed data visualization represents a bound protein. A statistical test may be performed using a dispersion model, represented by a black curve showing the probability distribution.

The multi-scale footprinting workflow 700 is continued in FIG. 7B. Varying the kernel width allows testing across size scales (e.g., large kernel, medium kernel, and small kernel), which enables DNA-binding proteins of varying sizes to be identified. This further enables predicting footprints from the sequence, decoding regulatory dynamics in different cell types, and identifying de novo motifs. The multi-scale footprinting workflow 700 demonstrates how the PRINT method combines statistical testing and deep learning approaches to analyze ATAC-seq data, enabling multi-scale footprinting analysis for predicting and interpreting regulatory element organization and protein-DNA interactions across diverse cellular contexts.

The bar plot 702 (FIG. 7A) compares the correlation between predicted and observed Tn5 bias for different computational approaches: 3-mer, 7-mer, position weight matrix (PWM), and convolutional neural network (CNN). The CNN approach demonstrates the highest correlation, indicating superior performance in predicting Tn5 bias.

FIG. 8 shows a schematic illustration 800 of the Tn5 bias prediction model. The schematic illustration 800 includes a BAC plasmid 802 containing naked DNA. The DNA sequence input 804 generated from the BAC plasmid 802 is represented as an initial matrix containing the nucleotides A, C, G, and T in a one-hot encoded format. The DNA sequence input 804 is passed through a convolutional layer 806, which applies a series of filters to detect sequence patterns. The output of the convolutional layer is processed through a ReLU activation 808, introducing non-linearity to the model. Following activation, the data undergoes a max-pool operation 810, which reduces the spatial dimensions of the processed data. The pooled data is then flattened before being input into a dense layer 812, represented by interconnected nodes. The final output is a predicted Tn5 bias 814, indicating an estimation of transposase bias along the DNA sequence input 804. This seq2PRINT framework demonstrates how the system transforms raw DNA sequence information into predicted bias measurements through multiple computational processing steps, enabling the analysis of DNA-protein interactions and regulatory elements at a sequence level.

PRINT identifies footprints across diverse scales of protein sizes with high sensitivity and specificity. A statistical approach was developed that quantifies the significance of depletion of observed Tn5 insertions relative to an estimated background dispersion at a given position to yield a footprint score, as will be further described in the methods section below. The approach reduces false positive detection on deproteinized DNA by an order of magnitude in contrast to prior footprinting methods. Inspired by earlier methods using MNase or ATAC-seq fragment sizes to infer DNA-bound proteins of different sizes, footprint scores were computed across window sizes ranging between 4-200 bp. This method was validated in vitro on deproteinized DNA incubated with purified MYC/MAX or CEBPA. Strong footprints were detected at TF motif sites only in the presence of the purified TF with very low background signal, whereas a well-established ATAC-seq footprinting method did not detect a distinction between foreground and background. Furthermore, an increase of footprints at low affinity sites at higher concentrations (100 nM vs 50 nM) of MYC/MAX was identified, suggesting footprint scores are sensitive to TF occupancy at a given site.

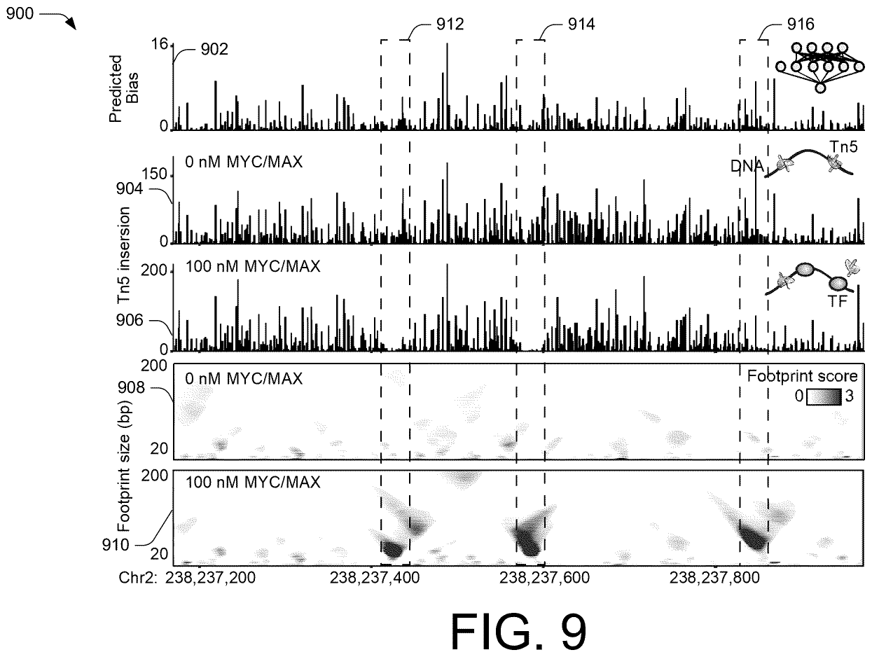

FIG. 9 depicts an example 900 of predicted Tn5 bias, observed Tn5 insertion, and multi-scale footprints on BAC DNA incubated with 0 nM or 100 nM MYC/MAX at example region chr2:238237173-238237972. A predicted bias plot 902 shows vertical bars representing predicted binding bias across a genomic region. A first insertion plot 904 and a second insertion plot 906 display Tn5 insertion patterns at different concentrations (0 nM and 100 nM MYC/MAX, respectively). A first footprint size plot 908 and a second footprint size plot 910 show footprint scores across different genomic positions. The example 900 highlights three specific regions of interest: first region 912, second region 914, and third region 916. These regions may correspond to areas of significant protein binding or regulatory activity. The plots are aligned vertically to allow direct comparison of features at corresponding genomic positions, with the x-axis showing chromosomal coordinates and the y-axis showing the respective measurement values. This visualization demonstrates how the PRINT method can detect and analyze protein-DNA interactions at different scales and under varying experimental conditions, providing insights into the genomic landscape of protein binding and regulatory elements.

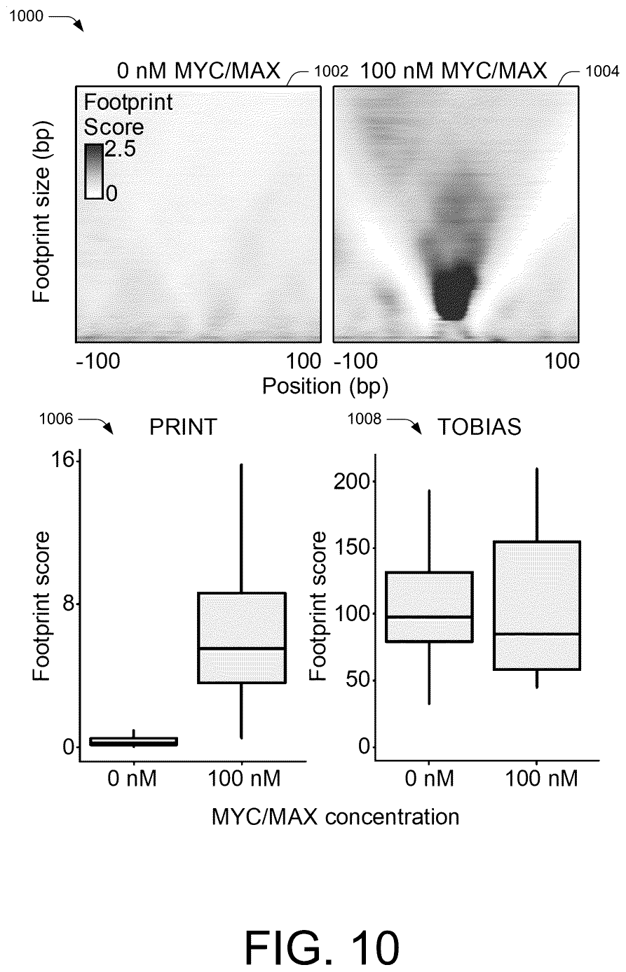

FIG. 10 depicts an example 1000 of aggregate multi-scale footprints on BAC DNA incubated with 0 nM or 100 nM MYC/MAX at MYC/MAX motif sites. A first footprint plot 1002 shows footprint scores at 0 nM MYC/MAX concentration, displaying a relatively uniform pattern with low intensity values. A second footprint plot 1004 shows footprint scores at 100 nM MYC/MAX concentration, revealing a more intense pattern with a distinct V-shaped region of high footprint scores. This comparison demonstrates how the presence of MYC/MAX proteins affects the footprint patterns at their binding motifs. A PRINT box plot 1006 displays a box plot comparing footprint scores between 0 nM and 100 nM MYC/MAX conditions using the PRINT method (n=275 positions). A TOBIAS box plot 1008 displays a box plot analysis showing the distribution of footprint scores across similar conditions using the TOBIAS method (n=158 positions). The TOBIAS method (e.g., Transcription factor Occupancy prediction By Investigation of ATAC-seq Signal) is an alternative framework for predicting transcription factor binding sites based on ATAC-seq data and is provided as a comparison for PRINT. The PRINT box plot 1006 and the TOBIAS box plot 1008 show the 1^{st}, 2^{nd} and 3^{rd} quartiles, while the whiskers of the PRINT box plot 1006 and the TOBIAS box plot 1008 extend to the farthest points falling within the 1st quartile-1.5 interquartile range (IQR) or 3^{rd} quartile+1.5 IQR. This visualization allows for a direct comparison between the PRINT and TOBIAS methods in detecting MYC/MAX binding events, highlighting the sensitivity of the PRINT method in capturing protein-DNA interactions at different protein concentrations.

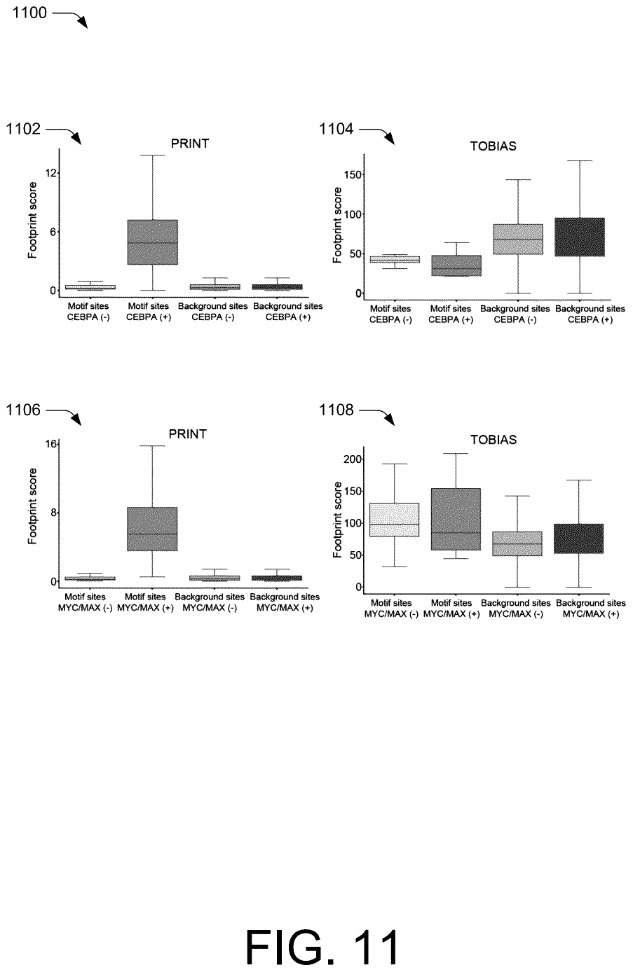

FIG. 11 depicts an example 1100 of box plots showing footprint scores at motif sites or background (non-motif) sites with 0 nM or 100 nM TFs. A first PRINT footprint plot 1102 displays footprint scores for CEBPA binding sites, showing distinct distributions between motif sites and background sites (n=207880). A first TOBIAS footprint plot 1104 presents a similar analysis using the TOBIAS method (n=104895). A second PRINT footprint plot 1106 shows footprint score distributions for MYC/MAX binding sites (n=207880). A second TOBIAS footprint plot 1108 displays the corresponding analysis using the TOBIAS method (n=104896). The first PRINT footprint plot 1102, the first TOBIAS footprint plot 1104, the second PRINT footprint plot 1106, and the second TOBIAS footprint plot 1108 show the 1^{st}, 2^{nd} and 3^{rd} quartiles. The whiskers of the first PRINT footprint plot 1102, the first TOBIAS footprint plot 1104, the second PRINT footprint plot 1106, and the second TOBIAS footprint plot 1108 extend to the farthest points falling within the 1^{st} quartile-1.5 interquartile range (IQR) or 3^{rd} quartile+1.5 IQR. This visualization allows for a direct comparison between the PRINT and TOBIAS methods in detecting CEBPA and MYC/MAX binding events, demonstrating the sensitivity of both methods in distinguishing between motif and background sites under different TF concentrations.

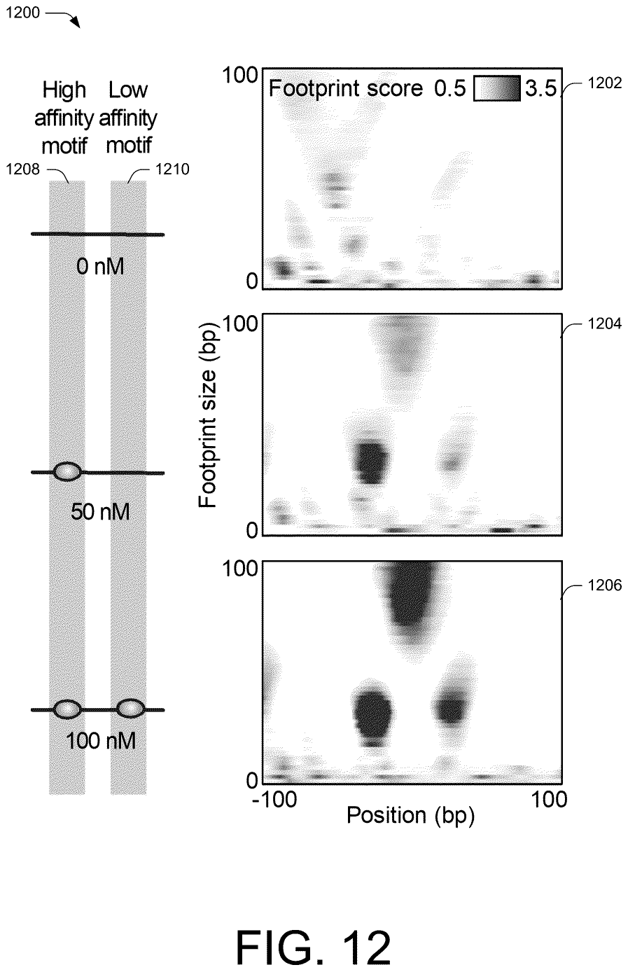

FIG. 12 depicts an example 1200 showing protein binding analysis at different concentrations. A first footprint score plot 1202 displays footprint scores across genomic positions at 0 nM protein concentration. A second footprint score plot 1204 shows footprint scores at 50 nM protein concentration. A third footprint score plot 1206 illustrates footprint scores at 100 nM protein concentration. A high affinity motif 1208 and a low affinity motif 1210 are indicated by gray vertical bars on the left side of the diagram, with binding events represented by ovals at the different concentrations. The example 1200 shows increasing intensity and clarity of binding patterns as the concentration increases from top to bottom, with the strongest footprint signals visible in the third footprint score plot 1206. This visualization demonstrates how the DNA-protein interaction analysis module 124 can detect and analyze protein binding events at different concentrations, revealing the relationship between protein concentration and the strength of footprint signals at both high and low affinity binding sites.

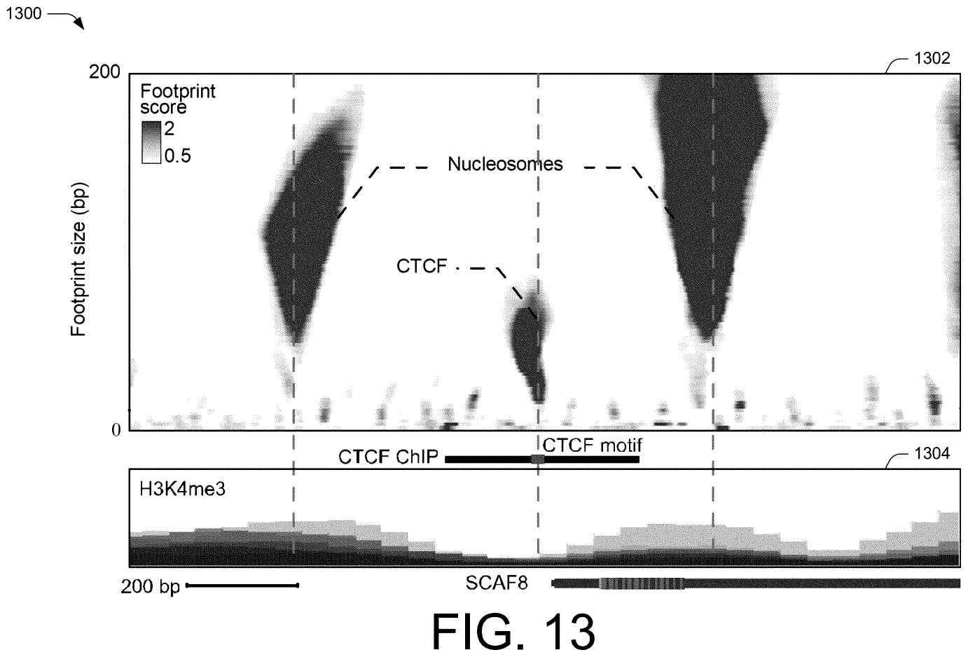

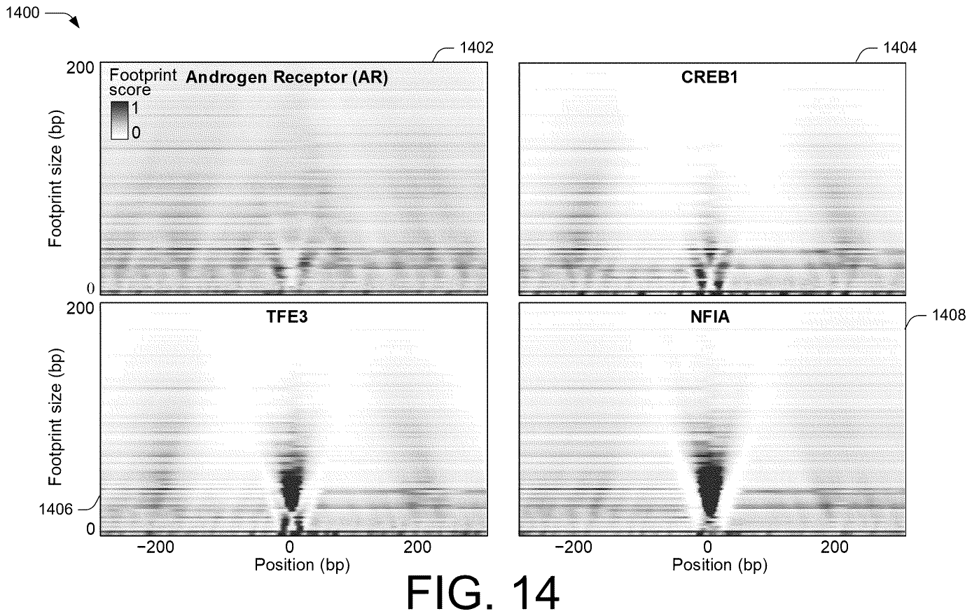

It was found that PRINT can detect footprints in mammalian cells. Distinct footprint patterns corresponding to nucleosomes and specific TFs were observed (FIGS. 13 and 14). TF binding patterns could be clustered into four representative categories. Consistent with previous studies using DNase I, footprint strength varied among TFs, including some that do not leave detectable footprints potentially due to weak or transient binding on DNA. Footprints can also be detected for repressor TFs (FIG. 14). Footprints were validated by benchmarking against ChIP-exo data, finding agreement at TF-bound sites and possible false negatives of ChIP-exo. Taken together, these results show that multi-scale footprinting with PRINT can robustly detect many distinct DNA-binding proteins.

FIG. 13 shows an example 1300 of multi-scale footprints in the cCRE region of chr6:154732971-154733770 in HepG2. A multi-scale footprint plot 1302 displays a pattern of protein binding across the genomic region, with darker regions indicating stronger footprint signals. The multi-scale footprint plot 1302 shows two prominent triangular patterns, with dashed lines indicating nucleosome regions. Between these, there is a smaller signal pattern labeled “CTCF,” which is a zinc finger protein that may be involved in gene regulation. Below the multi-scale footprint plot 1302, histone ChIP signals 1304 show the distribution of H3K4me3 histone modification mark across the same genomic region. The tracks are aligned vertically to allow direct comparison of features at corresponding genomic positions. At the bottom of the figure, a scale bar indicates a 200 bp length, and a label “SCAF8” indicates a position of the SCAF8 gene. The example 1300 demonstrates the relationship between nucleosome positioning, CTCF binding, and histone modifications at a specific genomic locus, illustrating the multi-scale nature of protein-DNA interactions that the PRINT method can detect.

FIG. 14 shows an example 1400 of aggregate multi-scale footprints for example TFs including AR, CREB1, TFE3, and NFIA. The example 1400 includes an androgen receptor footprint plot 1402 a CREB1 footprint plot 1404, a TFE3 footprint plot 1406, and an NFIA footprint plot 1408. Each plot displays footprint scores ranging from 0 to 1 on the y-axis, represented by varying intensities of shading, with darker shading indicating stronger footprint signals. The x-axis spans genomic positions from −200 to +200 base pairs relative to the binding sites. The example 1400 shows distinct binding patterns characteristic of each transcription factor, with the NFIA footprint plot 1408 and TFE3 footprint plot 1406 displaying particularly strong central footprint signals. This visualization demonstrates the ability of the DNA-protein interaction analysis module 124 to detect and differentiate binding patterns for various transcription factors, providing insights into the specific DNA-protein interactions occurring at different genomic locations.

### A DNA Sequence Model for Footprints

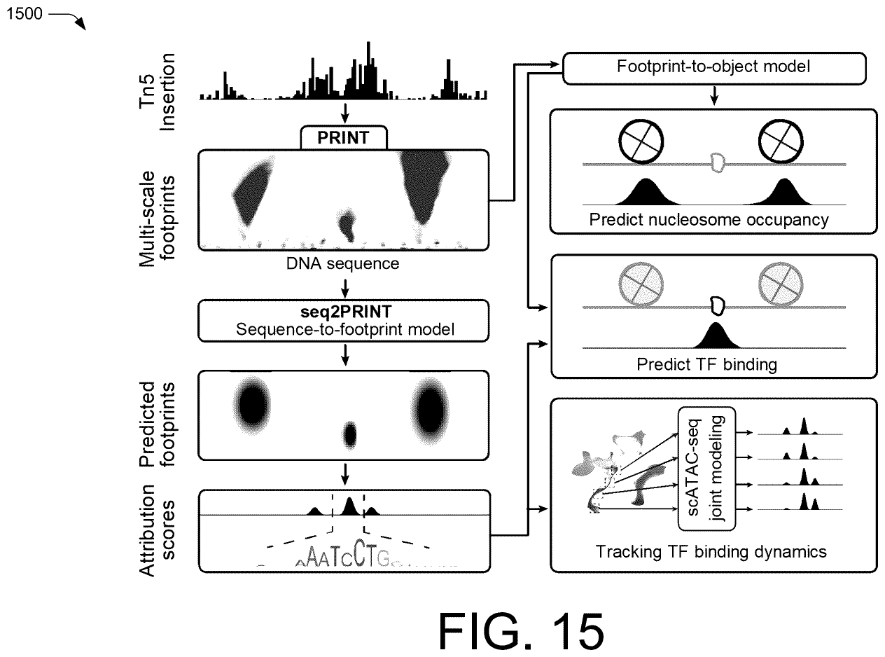

The multi-scale footprints were used to predict the binding of specific proteins to DNA. Models were designed that predict the binding of TFs and nucleosomes (FIG. 15). The nucleosome model uses multi-scale footprints as input to predict nucleosome summits mapped by nucleosome chemical mapping data; this model outperformed prior work.

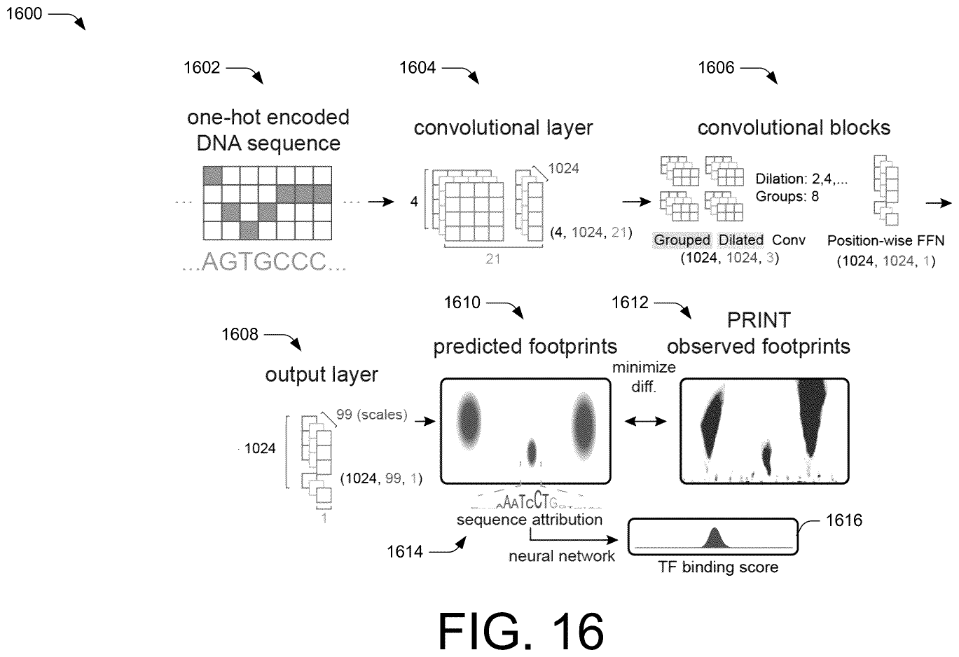

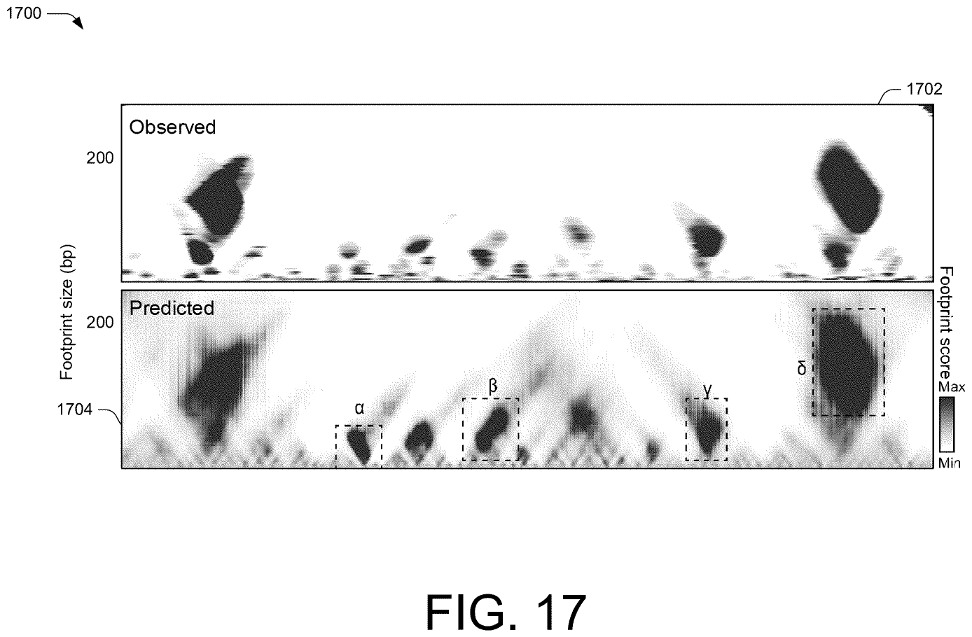

Building upon recent advances in deep learning, a model was created that uses a DNA sequence to predict multi-scale footprints (“seq2PRINT”) (FIGS. 15 and 16). The model was able to predict both nucleosome and TF footprints using local DNA sequence as the sole input (FIG. 17). An overall correlation of 0.75 was observed between predicted and observed multi-scale footprints in ATAC-seq data from HepG2 cells that was robust to sub-sampling of read depth.

The sequence features learned by seq2PRINT were extracted, and the resulting basewise DNA sequence attribution scores enabled the dissection of the TF binding architecture within a cCRE. In an example locus, attribution scores calculated with respect to the whole cCRE highlighted short sequences overlapping with TF motif positions across the region, while calculating scores for specific footprint objects highlighted specific motifs (FIG. 17). For footprints α and γ, the sequence model identified the motif underlying that footprint. For footprint β, the sequence model identified both the NFE2L2 motif underneath the footprint as well as a neighboring NFYB motif, indicating potential binding coordination between nearby TFs. Footprint δ likely represents a nucleosome and is predicted by nearby TF NRF1 and NFYB motifs, showing even longer-range dependencies and revealing the factors most associated with nucleosome positioning. The latter two examples further suggested that some TFs without a strong footprint (e.g., NFYB) can be detected by seq2PRINT, likely due to effects on neighboring elements, and that this approach could be used to model interactions between DNA-binding proteins within a cCRE.

FIG. 15 schematically shows an overview 1500 of footprint-to-object prediction and sequence-to-footprint prediction (seq2PRINT) models and their applications. The overview 1500 includes Tn5 insertion data at the top, which is processed through a PRINT module (e.g., the at least one footprinting model 128) to generate multi-scale footprints. The footprints and DNA sequence are then processed through a seq2PRINT module (e.g., the at least one sequence model 134), which functions as a sequence-to-footprint model. This generates predicted footprints (e.g., the predicted multi-scale footprint 330). Below the predicted footprints, attribution scores (e.g., the DNA sequence attribution scores 206) are displayed with a sequence motif visualization showing the relative importance of different nucleotides. The overview 1500 branches into three parallel analysis paths on the right side. The first path leads to a footprint-to-object model that predicts nucleosome occupancy, with corresponding density plots below. The second path connects to a model that predicts transcription factor (TF) binding, with an associated density plot. The third path leads to a tracking module for TF binding dynamics. The overview 1500 shows the progression from raw insertion data (e.g., the ATAC-seq data 118) through multiple analytical steps to generate predictions about protein-DNA interactions at different scales. Arrows indicate the flow of data between components, with each step transforming or analyzing the data in different ways to characterize various aspects of chromatin organization.

FIG. 16 shows an overview of footprint-to-nucleosome prediction. Tracks showing observed Tn5 insertion, nucleosome occupancy predicted by NucleoATAC and the footprint-to-nucleosome model, and ground truth nucleosome occupancy measured by chemical mapping are shown. Precision-recall curves of NucleoATAC and PRINT for nucleosome mapping are shown. Predicted nucleosome centers are considered to be a true positive prediction if it is within 75 bp (c) or 50 bp (d) from the nearest ground truth nucleosome center. Line plots showing the relationship between model performance and local coverage or distance to nearest cCRE are also shown.

FIG. 16 shows a schematic illustration 1600 of the seq2PRINT model. The schematic illustration 1600 includes a one-hot encoded DNA sequence 1602, which represents DNA sequence data in a matrix format with nucleotides encoded as binary values. The one-hot encoded DNA sequence 1602 is input into a convolutional layer 1604, which processes the one-hot encoded DNA sequence 1602. The output from the convolutional layer 1604 then passes through convolutional blocks 1606, which contain multiple layers with parameters including dilation and grouping values. The processed data moves through an output layer 1608, which generates predicted footprints 1610. The predicted footprints 1610 are compared with PRINT observed footprints 1612 to minimize differences between the predictions (e.g., the predicted footprints 1610) and observations (e.g., the PRINT observed footprints 1612) during training. The schematic illustration 1600 shows a sequence attribution 1614 step that analyzes DNA sequence patterns of the predicted footprints 1610. A TF binding score 1616 is generated by a neural network (e.g., the at least one TF binding prediction model 138), which quantifies the likelihood of transcription factor binding at specific locations along the one-hot encoded DNA sequence 1602. The schematic illustration 1600 demonstrates how an initial DNA sequence is processed to generate predictions about protein-DNA interactions. Arrows indicate the directional flow between components, showing how the data is transformed at each step.

FIG. 17 shows an example 1700 comparing observed and predicted multi-scale footprints in the example region chr4:39181940-39182739. An observed footprint plot 1702 displays a series of regions of varying sizes across a horizontal axis, representing experimental data of protein-DNA interactions. The predicted footprint plot 1704 shows the computational predictions made by the PRINT method described herein. In the predicted footprint plot 1704, there are several distinct footprints labeled with Greek letters (α, β, γ, δ) that correspond between the observed and predicted panels, demonstrating matching between the observed footprint plot 1702 and the predicted footprint plot 1704. Accordingly, the example 1700 demonstrates how the PRINT method can accurately predict protein-DNA interaction patterns that match experimental observations.

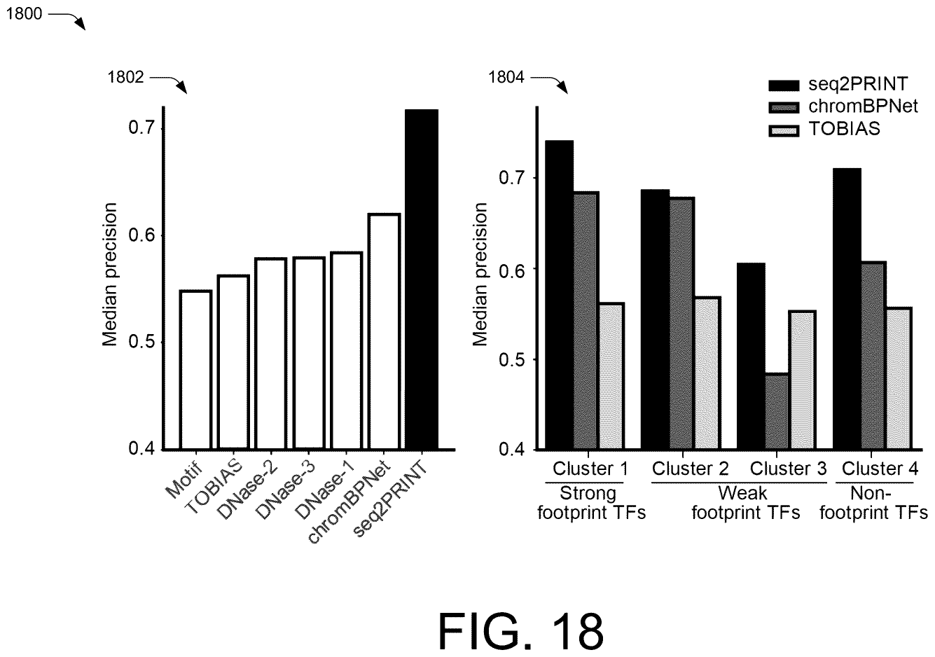

The seq2PRINT approach described herein can be used to predict the binding of TFs genome-wide. The sequence attribution scores from seq2PRINT were used to generate a TF binding score trained to predict ChIP-seq data (see the Methods section below). The TF binding score was able to predict TF binding with high precision and outperformed previous methods (FIG. 18). TFs with weak or no direct footprint were able to be predicted where other methods had particularly low performance (FIG. 18, FIG. 13). To investigate how the model was detecting these TFs, the effects of loss of TF binding were simulated by scrambling their motif sequences. The model predicted changes to nearby footprints across scales such as nucleosomes, which correlated well with experimentally determined footprint changes such as degron-induced CTCF depletion (aggregate R=0.93, median per-locus R=0.66). Similar results were obtained with dexamethasone-induced glucocorticoid receptor relocation and DNA binding, as well as IFN-induced Stat2 binding to DNA. This approach was applied broadly and TFs with strong effects on nucleosomes flanking the TF (e.g., JUN, YY1) or at the same position of the TF (e.g., ZKSCAN1) were identified. The predicted changes in footprint score were highly similar among TF families in a manner largely independent of binding site similarity.

The seq2PRINT attribution scores identified DNA sequence patterns predictive of footprints, enabling the identification of motifs de novo. Using the trained model, local sequence attribution scores were clustered and aligned, and 106 de novo motifs were identified. These de novo motifs recovered known motifs in an unbiased fashion as well as composite motifs such as dimers of SOX. Several de novo motifs were associated with strong TF or nucleosomal footprints despite no significant match to a known motif database.

FIG. 18 depicts an example 1800 comparing different computational methods for analyzing DNA-protein interactions. A precision comparison plot 1802 shows a bar graph comparing the median precision performance of multiple methods including Motif, TOBIAS, DNase-1, DNase-2, DNase-3, chromBPNet, and seq2PRINT. The seq2PRINT method demonstrates the highest median precision score among all compared methods. A cluster comparison plot 1804 displays a bar graph comparing the median precision performance of three different computational methods (seq2PRINT, chromBPNet, and TOBIAS) across four different clusters labeled on the x-axis. The clusters are divided into “Strong-footprint TFs” (Cluster 1), “Weak-footprint TFs” (Clusters 2 and 3), and “Non-footprint TFs” (Cluster 4). The y-axis shows the median precision values. Seq2PRINT consistently shows the highest performance across all clusters, with particularly strong performance in Clusters 1 and 4. ChromBPNet shows similar but slightly lower performance compared to seq2PRINT in Clusters 1 and 2, but underperforms in Clusters 3 and 4. TOBIAS demonstrates the lowest performance across all clusters. This comparison illustrates the superior performance of seq2PRINT in predicting transcription factor binding across different types of TFs and footprint strengths compared to the other computational methods.

cCREs Restructure Across Hematopoiesis

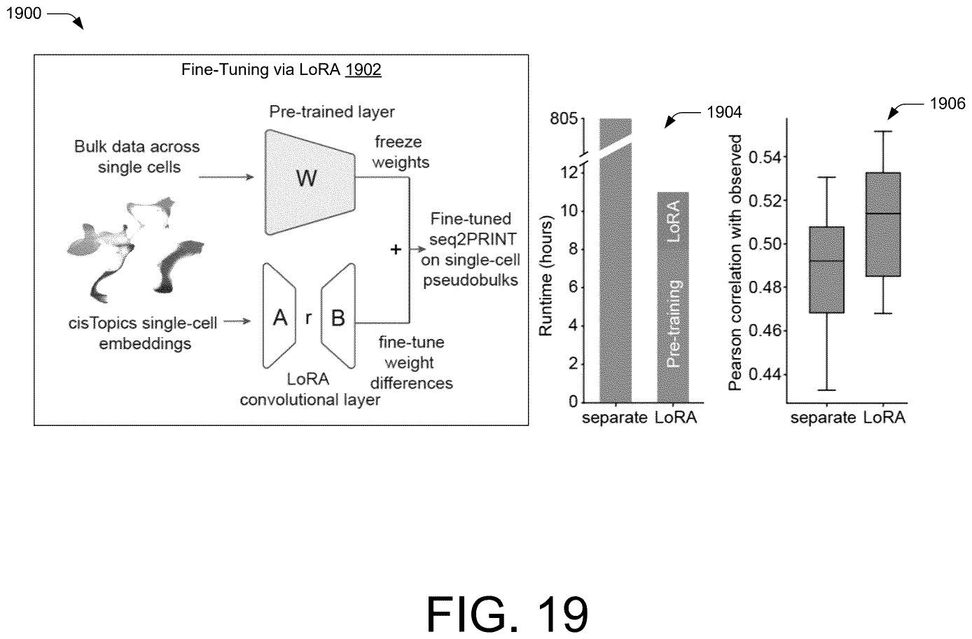

Multi-scale footprinting and seq2PRINT resolved the dynamics of TF binding across hematopoiesis. SHARE-seq was used to generate joint single-cell ATAC-seq and RNA-seq datasets for 874,480 bone marrow mononuclear cells from seven human donors. To enable footprinting, single cells were merged into 1,000 pseudo-bulks representing all major cell types and developmental transitions. A central challenge in applying deep-learning sequence models is that computational intensity scales poorly with the number of cell types or conditions. To overcome this challenge, a common model was trained across all the data representing 2.2 billion reads, and low-rank adaptation of large models (LoRA) was used to fine-tune a sequence model for each pseudo-bulk, achieving ˜80-fold speed improvement and increased prediction accuracy as compared to training separate models for each pseudo-bulk (FIG. 19).

FIG. 19 shows an overview 1900 that schematically illustrates using LoRA to scale up seq2PRINT training on large numbers of samples (pseudo-bulks). The overview 1900 shows a system architecture that incorporates fine-tuning via LoRA 1902, which processes bulk data across single cells and includes a pre-trained layer with frozen weights. A LoRA convolutional layer that processes cisTopics single-cell embeddings through fine-tuned weight differences. The overview 1900 further shows a runtime comparison plot 1904 comparing run time of training separate seq2PRINT models on 1000 pseudo-bulks versus using LoRA fine-tuning, with values shown on a scale from 0 to 12 hours for the fine-tuning via LoRA 1902 versus a scale from 0 to 805 hours for training the separate seq2PRINT models. Boxes show 1^{st}, 2^{nd} and 3^{rd} quartile. Whiskers show the farthest point falling within the 1^{st} quartile-1.5 IQR or 3^{rd} quartile+1.5 IQR. The correlation plot 1906 shows the Pearson correlation coefficient between predicted and observed footprints by either training separate seq2PRINT models on 1000 pseudo-bulks versus using LoRA fine-tuning. As such, the fine-tuning via LoRA 1902 is computationally efficient and accurate compared to training separate seq2PRINT models.

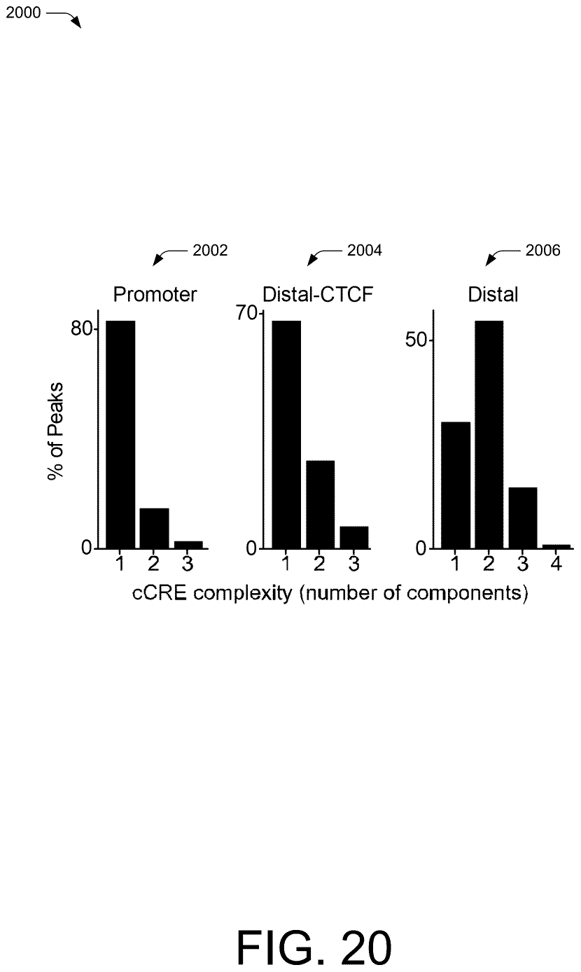

TF binding predictions revealed distinct groups of TFs bound at the same cCRE across cell types. At the promoter for SPI1 (PU.1), a myeloid master regulator, seq2PRINT binding scores were high at SPI1 and AP-1 sites in myeloid cells, while only strong GATA1 binding was predicted in erythroid cells, consistent with the known regulatory relationships of these TFs. Notably, these distinct TF binding patterns and those at other loci are not distinguishable solely by measuring the overall accessibility of the promoter. The complexity of TF binding patterns at each cCRE across the genome was quantified using principal component analysis, and it was found that complex cCREs (>1 component) were highly enriched at distal cCREs relative to promoters (69.8% versus 17.2%, FIG. 20), highlighting the cell type-specific utilization of enhancers.

FIG. 20 depicts an example 2000 showing a cCRE complexity analysis across different genomic regions. The example 2000 includes a promoter plot 2002, a distal CTCF plot 2004, and a distal plot 2006. Each plot shows the percentage of peaks on the y-axis and the cCRE complexity (number of components) of TF binding modes on the x-axis, with bars representing the distribution of complexity across different genomic regions. shows a percentage of all cCREs with the given complexity. Individual cCRE binding patterns were decomposed into principal components, and complexity was defined as the number of components needed to explain 98% of variance. The example 2000 demonstrates how the DNA-protein interaction analysis module 124 can characterize the complexity of cis-regulatory elements (cCREs) in various genomic contexts, providing insights into the organization and potential functional roles of these regulatory regions.

Analysis of TF binding along the erythroid differentiation trajectory demonstrated that the establishment of cCREs occurs sequentially. This is exemplified by the HS3 enhancer within the hemoglobin locus control region (LCR). In hematopoietic stem cells (HSCs) and common myeloid progenitors, nucleosomes were unphased, and low TF binding wase predicted. As cells progressed along erythroid development, as ordered by pseudo-time, and expression of HBB (β-hemoglobin) increased, nucleosome footprints first became phased at the edge of the cCRE with strong TF binding scores at GATA/TAL motif sites. The cCRE then progressively widened, with the addition of KLF1/NFE2 factor binding at the edge. The HBB promoter exhibited the same sequential TF binding patterns, and PRINT/seq2PRINT binding predictions at the locus corresponded well with published massively parallel reporter assay data.

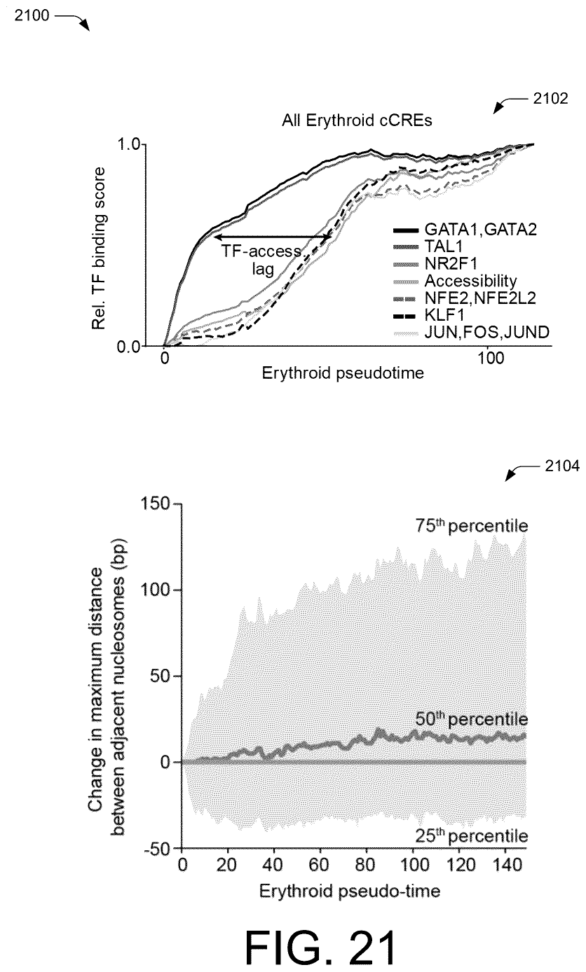

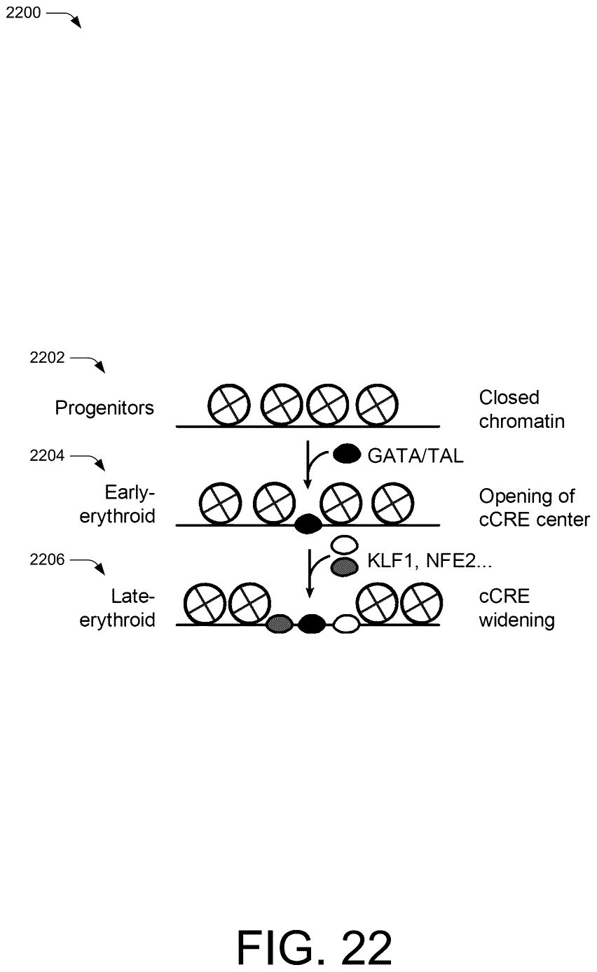

This pattern of cCREs extending outwards from a central TF across erythroid-associated cCREs was found genome-wide. TF binding scores for GATA and TAL factors increased early in erythroid pseudo-time, while appreciable overall cCRE opening and binding at NFE2, KLF1, NR2F1, and AP-1 factors occurred later during differentiation (FIG. 21). TFs predicted to bind before the gain in accessibility and target gene expression also bound closer to the cCRE center, while later-binding TF motifs localized to flanking regions (FIG. 22). These observations were largely independent of cCRE complexity, which is in line with previous studies showing stereotypical TF motif arrangements within cCREs and enrichment of chromatin remodelers at cCRE edges. This analysis was repeated on the B-cell differentiation trajectory, and the same sequential establishment of cCREs with different central and flanking TFs was found. These findings connect cCRE restructuring and TF binding during in vivo differentiation, suggesting core- and flank-binding TFs might contribute differentially to cCRE establishment, expansion, and gene expression.

FIG. 21 shows an example 2100 analysis of TF binding scores of representative TFs within erythroid cCREs across the pseudo-time of erythroid differentiation. An erythroid cCREs plot 2102 includes multiple curves representing different transcription factors and accessibility measurements over erythroid pseudotime (x-axis), with the y-axis corresponding to a relative TF binding score. A change in nucleosome distance plot 2104 shows the variation in maximum distance between adjacent nucleosomes measured in base pairs (bp) (y-axis) across erythroid pseudo-time (x-axis), displaying 25^{th}, 50^{th}, and 75^{th} percentile ranges. The example 2100 demonstrates how the DNA-protein interaction analysis module 124 can track changes in regulatory element activity and nucleosome organization throughout erythroid cell differentiation, which may provide insights into the dynamic nature of chromatin structure during cellular development.

FIG. 22 illustrates an example 2200 showing the stepwise progression of chromatin accessibility and transcription factor binding during erythroid cell differentiation. The example 2200 shows the sequential binding of TFs and widening of cCREs during differentiation, including the progression from progenitor cells through early-erythroid to late-erythroid stages. In a progenitors binding stage 2202, the chromatin is shown in a closed state with four crossed circles (representing nucleosomes) positioned along the DNA, indicating compact chromatin structure. During an early-erythroid binding stage 2204, a GATA/TAL transcription factor (shown as a black oval) binds to the DNA, initiating the opening of the cCRE center. The nucleosome pattern remains similar but shows initial signs of reorganization around the GATA/TAL binding site. In a late-erythroid binding stage 2206, additional transcription factors including KLF1 and NFE2 (shown as a white oval and a gray oval, respectively) bind to the DNA, leading to further widening of the cCRE. The nucleosomes have become more widely spaced, as indicated by the increased distance between the crossed circles, demonstrating the progressive opening and expansion of the cCRE. Thus, the example 2200 shows how sequential binding of specific transcription factors coordinates with nucleosome repositioning to establish and maintain open chromatin regions during erythroid differentiation.

### De Novo Motifs Characterize Aging HSCs

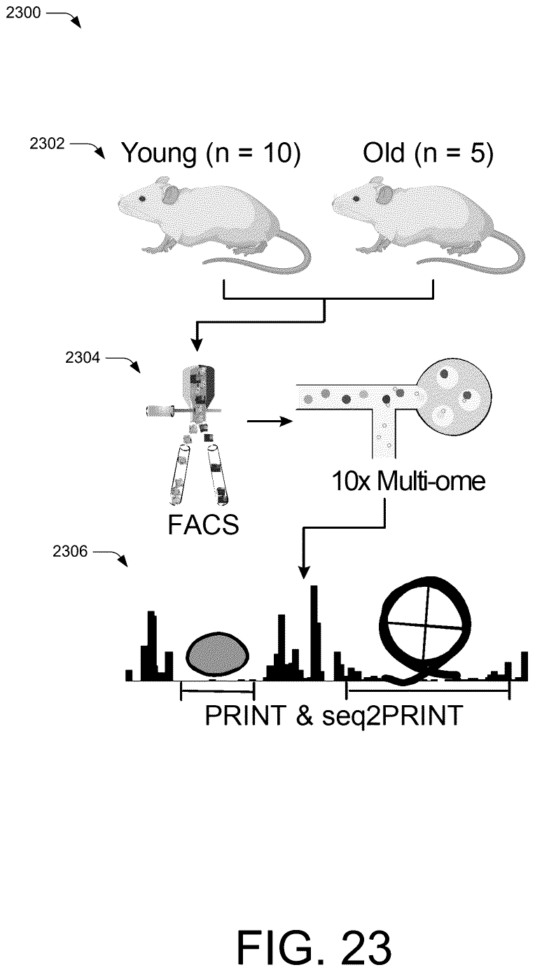

Seq2PRINT was utilized to analyze changes in cCRE organization during aging. Biological aging, a multifactorial process affecting the physiology of a broad range of tissues, includes notable changes to function and proliferation of HSCs. Previous studies have indicated that aging is accompanied by widespread epigenetic changes. It was hypothesized that seq2PRINT would be suitable for detecting differences in TF activity and cCRE structure across aging. Hematopoietic progenitor cells (Lineage-) and HSCs (Lineage⁻ Sca-1⁺ c-Kit⁺ CD48⁻ CD150⁺) were isolated from the bone marrow of young (11 weeks old, n=10) and aged (24 months old, n=5) male mice by FACS, and joint ATAC-RNA profiling of 48,225 cells covering 14,640 HSCs and 33,585 hematopoietic progenitor cells was obtained using the 10× Multiome platform (FIG. 23). HSCs from old and young mice clustered separately and matched previously reported aging signatures and marker genes. To further explore HSC heterogeneity, one-hundred representative cell states among HSCs were identified to generate pseudo-bulks. HSC states were identified by collecting previously described gene sets covering lineage bias and aging as well as gene programs learned from the data. Scoring and clustering of HSC pseudo-bulks using these gene sets identified five HSC subpopulations distinguished by age and lineage potential. These five HSC cell states represent major categories that are consistent with prior findings and can be further partitioned, suggesting additional axes of age-associated variation (e.g., oxidative phosphorylation regulation, unfolded protein response) that may be relevant to the pathogenesis of the aged immune system.

FIG. 23 shows a schematic overview 2300 of data generation of HSC and progenitor cells. The schematic overview 2300 includes mouse groups 2302, including two groups of mice: young mice (n=10) and old mice (n=5). A FACS analysis 2304 is performed, which includes flow cytometry followed by a multi-ome analysis. A footprint analysis 2306 is performed on data generated via the multi-ome analysis, which includes PRINT and seq2PRINT analysis results.

It was found that cCREs undergo extensive changes to TF binding upon aging. Seq2PRINT was applied on all HSC pseudo-bulks, and young and old HSCs were compared to find a strong increase in NF-I, Runx, Ets, Gata, and AP-1 (e.g., Fos, Jun) activity. Considering that the comparison of all young and old HSCs might be confounded by changes in HSC subpopulation composition during aging, this analysis was performed using only Mk-biased and multi-lineage subpopulations, respectively. Subpopulation-specific comparison yielded overall similar results, indicating the majority of changes are shared across subpopulations. However, a subset of Ets motifs, especially Spil/Spib/Spic and Elf motifs, displayed age-associated down-regulation specifically in the multi-lineage subpopulation, indicating subpopulation-specific TF changes in aging.

To examine the sequence motifs learned by seq2PRINT in an unbiased and comprehensive manner, de novo motifs with differential activity were identified, including the loss of many CG-rich de novo motifs in aging, possibly related to DNA-binding factors recognizing methylation changes characteristic of aging in a variety of contexts. An increase in activity at composite motifs containing an Ets homo-dimer or hetero-dimer with Gata, AP-1, and Runx motifs was found. Further, Ets/Runx composite motifs were particularly enriched in old multi-lineage HSCs. Supporting this finding, prior work has proposed a role for Ets/Runx co-occurrence in HSC maintenance and myeloid fate. Mechanistically, Ets binding of DNA is negatively regulated by an autoinhibitory domain that is released upon co-binding with Runx or second Ets-family TF, and physical interactions have been shown in two experimentally determined structures (PDB: 4L0Z, 2NNY). To test whether seq2PRINT-predicted Ets composite motifs could represent similar direct interactions, AlphaFold3 was used to predict structures, and it was found that all seq2PRINT-identified Runx/Ets configurations showed a similar physical interaction regardless of orientation. In support of the validity of the predictions, AlphaFold3 structures based on the known Ets/Ets and Ets/Runx motif configurations were highly concordant with the experimentally measured structure (RMSD between predicted structures for motif 10 and PDB 4L0Z, 0.825 Å), and solvent-inaccessible bases at the interface with each TF matched the correct core motif. Seq2PRINT thus reveals rewiring of TFs during aging, and, through de novo motif analysis complemented by AlphaFold3, predicts a wide diversity of Ets and Runx co-binding arrangements implicated in aging and HSC self-renewal.

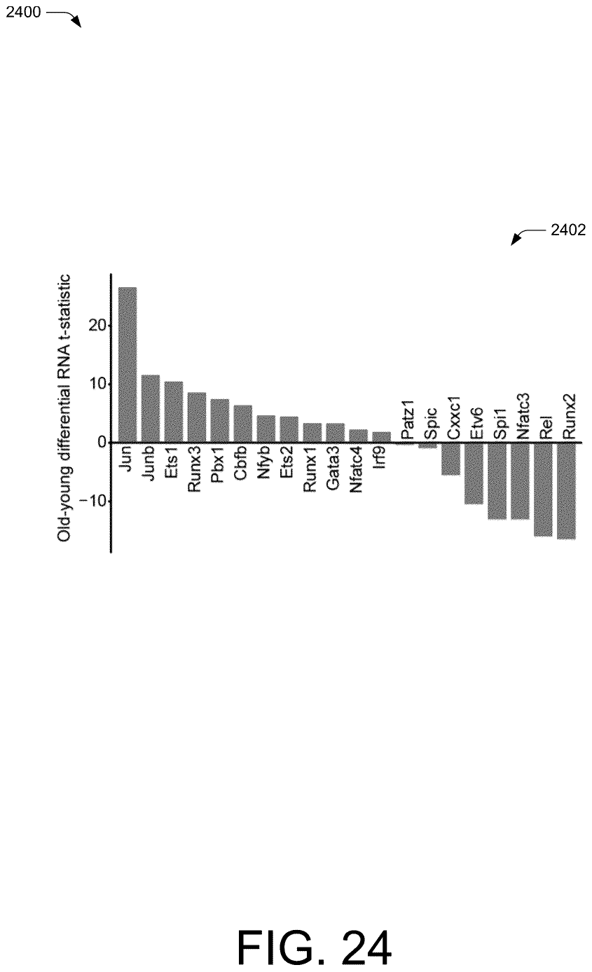

FIG. 24 depicts an example 2400 showing differential RNA expression analysis results for partner TFs forming composite motifs with Ets1. The example 2400 includes a differential RNA expression plot 2402 displaying differential expression values for partner TFs forming composite motifs with Ets1, with Jun showing the highest positive differential expression and Runx2 showing the lowest negative differential expression among the measured TFs for old-young differential RNA t-statistic.

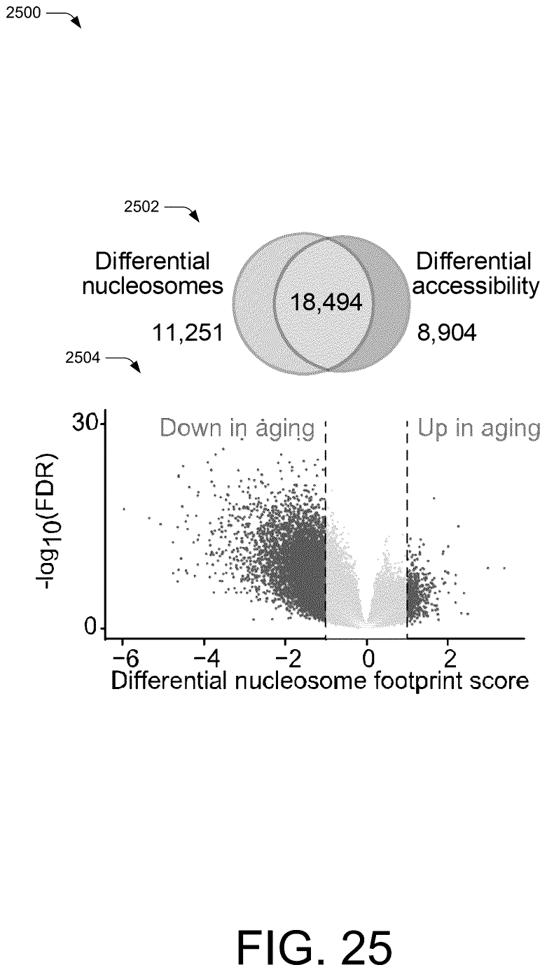

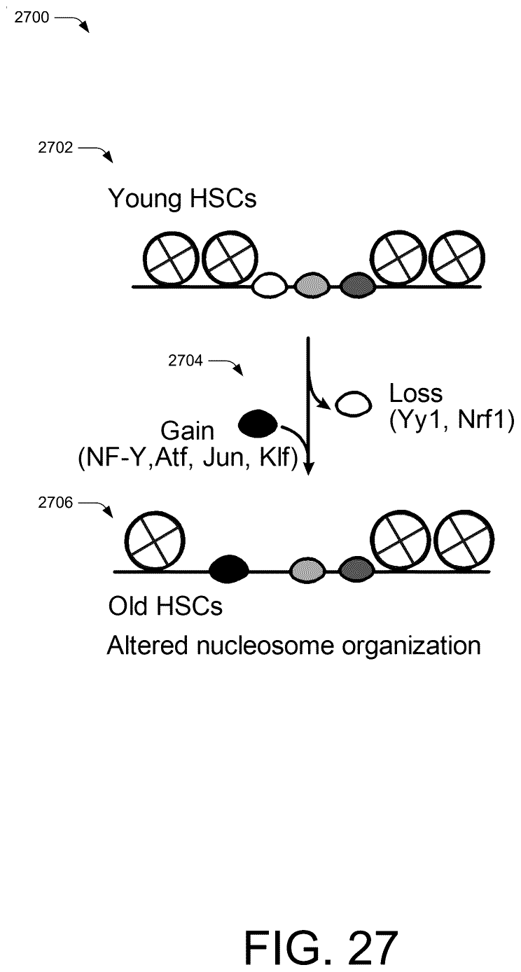

Across multiple systems, studies have described a global loss of nucleosomes associated with aging and senescence, although debate remains as to whether the loss is global or restricted to specific TF-associated sites. To further explore the epigenetic decline of HSCs, PRINT was used to measure nucleosome occupancy across cCREs during aging. A widespread loss of nucleosome footprints across cCREs in old HSCs was observed (FIG. 25). It was noted that 38% of cCREs with differential nucleosome footprints did not show significant accessibility changes, highlighting the utility of footprinting to resolve changes. A reduction in nucleosome footprints does not necessarily indicate a loss of nucleosome number across the genome, however, as this could also result from loss of nucleosome positioning/phasing, and the analysis is mainly limited to cCREs. To determine whether specific TFs were altered at these loci, seq2PRINT motifs were used and strong enrichment for a loss of TFs such as Yy1, Nrf1 and Ctcf and gain of Nfyb, Atf, Jun, Klf was found. Notably, Yy1, Nrf1 and Ctcf were also predicted to regulate nucleosome position in diverse cell lines. Yy1 has been previously shown to be a regulator of HSC self-renewal and quiescence. Overall, widespread alteration of nucleosome footprints and rewiring of TF binding across aging was found, suggesting a dysregulation of cCRE structure in aging (FIG. 27).

FIG. 25 shows an example 2500 of age-associated nucleosome changes. The example 2500 includes a Venn diagram 2502 of the overlap between cCREs with differential nucleosomes and cCREs with differential accessibility during aging, and a volcano plot 2504 of differential nucleosome changes during aging, which are shaded by density. The volcano plot 2504 displays differential nucleosome footprint scores on the x-axis versus −log₁₀(false discovery rate) on the y-axis, with data points separated into “down in aging” and “up in aging” regions by vertical dashed lines. The example 2500 thus demonstrates the relationship between changes in nucleosome positioning and chromatin accessibility during aging, as analyzed by the DNA-protein interaction analysis module 124.

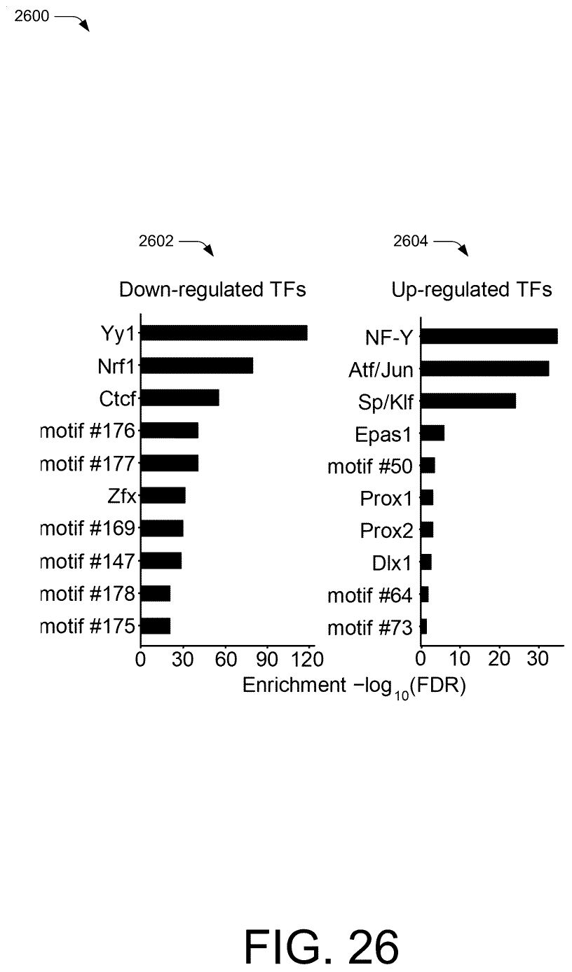

FIG. 26 shows an example 2600 of TF motif enrichment at nucleosome footprints lost during aging (absolute difference in footprint score >1 and FDR<0.01). A down-regulated transcription factors plot 2602 includes TFs Yy1, Nrf1, Ctcf, and Zfx, and several numbered motifs (176, 177, 169, 147, 178, and 175), with their enrichment scores shown on the x-axis ranging from 0 to 120. An up-regulated transcription factors plot 2604 shows another set of transcription factors and motifs, including NF-Y, Atf/Jun, Sp/Klf, Epas1, Prox1, Prox2, and Dlx1, and additional numbered motifs (50, 64, and 73), with enrichment scores ranging from 0 to 30. The top ten enriched motifs that are up-regulated (the up-regulated transcription factors plot 2604) or down-regulated (the down-regulated transcription factors plot 2602) in aging are shown. The example 2600 demonstrates the changes in regulatory patterns between different conditions or cell states.

FIG. 27 shows an overview 2700 schematically illustrating age-associated cCRE reorganization. The overview 2700 is organized vertically in three stages, showing the progression from young to old HSCs. A young HSC state 2702 depicts four circular objects with X-marks (representing nucleosomes) arranged along a DNA region, with three small oval shapes between them representing transcription factors. The young HSC state 2702 represents the initial nucleosome organization in young HSCs. A transition state 2704 illustrates a loss event showing the removal of transcription factors Yy1 and/or Nrf1 (represented by a small white oval), and a gain event showing the addition of transcription factors NF-Y, Atf, Jun, and/or Klf (represented by a black oval). An old HSC state 2706 shows three nucleosomes and different transcription factors, demonstrating the altered nucleosome organization that occurs with aging. The overview 2700 illustrates how the chromatin landscape changes during HSC aging, with specific transcription factors being lost and gained, accompanied by changes in nucleosome positioning and organization.

### DISCUSSION

The results demonstrate complex dynamics of TF binding and nucleosome repositioning at cCREs across cell differentiation and aging. Prior footprinting studies have suggested that TF binding is mostly determined by wholesale opening or closing of cCREs instead of differential binding of TFs within the same cCRE. In contrast, by using the techniques described herein, it was shown that cCREs are occupied by distinct sets of TFs across cell types. This is exemplified by the SPI1/PU.1 and Wasl promoter analysis above in which multiple configurations of nucleosomes and TFs are observed despite similar levels of overall accessibility, revealing mechanisms of gene regulation that would be missed by standard chromatin accessibility analysis. In direct support of this model, studies mapping TF binding by ChIP-seq report that TFs switch in development. Along continuous trajectories of hematopoietic differentiation, cCREs widen sequentially around central TFs, with flanking TFs binding at later stages of development. This suggests that the establishment of enhancers is an analog (e.g., operating on a continuum) rather than a digital (e.g., binary “on” or “off”) process.

More broadly, PRINT generates an image of all DNA-binding proteins simultaneously in a given cell population. Modeling footprints with seq2PRINT infers TF binding regardless of its direct footprint strength, enables de novo identification of TF motifs and cooperative binding, and suggests TF have specialized functions such as remodeling or stabilizing neighboring nucleosomes. These attributes contrast seq2PRINT with ChromBPNet, which is tuned to predict accessibility rather than interpret the sequence features underlying footprints. Using LoRA, the computational burden of footprint prediction is reduced, enabling the extension of seq2PRINT to single-cell epigenomics data. This approach is anticipated to enable methods that connect high-resolution footprinting to diverse epigenomic data types, such as genome structure and local gene expression. Similarly, identification of TF binding and attribution of footprints to specific sequences at base-pair resolution may also ascribe new functions to disease-causing genetic variations previously obscured by peak-based analyses.

It is envisioned that PRINT may be used in combination with other methods, such as methyltransferase-based single-molecule footprinting and DNA sequence mutagenesis assays, to further dissect the structure and function of specific cCREs. However, as ATAC-seq has been broadly adopted and widely used for single-cell assays, seq2PRINT may enable both retrospective and prospective studies that atlas footprints across a broad range of healthy and diseased human tissues. Taken together, the approach described herein extracts a rich multidimensional feature space from unidimensional chromatin accessibility data in order to reveal the dynamic structure of cCREs across high genomic and cell-state resolution.

### Methods

### Cell Culture

HepG2 cells were cultured in Dulbecco's Modified Eagle Medium (DMEM) with the addition of 10% FBS and 1% of penicillin-streptomycin. Cells were incubated at 37° C. in 5% CO₂ and maintained at the exponential phase. Cells were digested with TrypLE express for preparing a single-cell suspension.

### BMMC Sample Processing

Frozen human Bone Marrow Mononuclear Cells (BMMCs) were thawed in a 37° C. water bath for 1 minute and transferred to a centrifuge tube. Pre-warmed DMEM with 10% FBS was added to cells drop-wisely. The cells were spun at 400 times the acceleration due to gravity for 3 minutes at room temperature. After removing the supernatant, the cells were washed twice in phosphate-buffered saline (PBS) with 0.04% BSA. To deplete neutrophils, the cells were resuspended in chilled Dulbecco's PBS (DPBS) with 0.2% BSA and human TrueStain FcX and incubated on ice for 10 minutes to reduce non-specific labeling. The cells were then incubated on ice for another 30 minutes after adding a biotin-conjugated anti-human CD15 antibody. After immunostaining, superparamagnetic beads (e.g., MyOne T1 beads) were added to the sample to capture the neutrophils for 5 minutes at room temperature. DPBS with 0.2% BSA was then added to dilute the sample. The sample was placed on a magnet for 3 minutes, and an aliquot of the sample (e.g., 1 milliliter) was transferred to a new tube while the sample was on the magnet. The cells were then ready for fixation and the SHARE-seq experiment, as described below.

### Fixation

Cells were centrifuged at 300 times the acceleration due to gravity for 5 minutes and resuspended to 1 million cells per milliliter in PBS with iodide (PBSI). Cells were fixed by adding formaldehyde to a final concentration of 1% and incubated at room temperature for 5 minutes. The fixation was stopped by adding 56.1 microliters (μL) of 2.5 molar (M) glycine, 50 μL of 1 M Tris-HCl pH 8.0, and 13.3 μL of 7.5% BSA on ice. The sample was incubated at room temperature for 5 minutes and then centrifuged at 500 times the acceleration due to gravity for 5 minutes to remove supernatant. All centrifugations were performed on a swing bucket centrifuge. The cell pellet was washed twice with PBSI and centrifuged at 500 times the acceleration due to gravity for 5 minutes between washings. The cells were resuspended in PBS with 0.1 units per microliter (U/μL) Enzymatics RNase Inhibitor and aliquoted for transposition.

### SHARE-Seq

Following fixation, SHARE-seq was performed as previously described, with the following modifications. To improve transposition, transposition was performed using a pre-assembled transposon reagent (Tn5). To improve RNA capture, polyadenine (polyA) was added to transcripts prior to reverse transcription. To do this, transposed cells (60 μL) were mixed 240 μL of polyA mix (final concentration of 1× Maxima RT buffer, 0.25 U/μL Enzymatics RNase Inhibitor, 0.25 U/μL SUPERase RI, 0.018 U/μL E. coli polyA enzyme, 1 mM rATP). The sample was aliquoted to 50 μL per PCR tube and incubated at 37° C. for 15 minutes.

### Quantification and Sequencing

Both scATAC-seq and scRNA-seq libraries were quantified with the KAPA Library Quantification Kit and pooled for sequencing. Single cell libraries were sequenced on the Nova-seq platform (Illumina) using a 200-cycle kit (Read 1: 50 cycles, Index 1: 99 cycles, Index 2: 8 cycles, Read 2: 50 cycles). Bulk libraries were sequenced on the Nova-seq platform (Illumina) using a 100-cycle kit (Read 1: 50 cycles, Index 1: 8 cycles, Index 2: 8 cycles, Read 2: 50 cycles).

### SHARE-Seq Data Pre-Processing

SHARE-seq data were processed using the SHARE-seqV2 alignment pipeline and aligned to hg38. Open chromatin region peaks were called on individual samples using MACS2 peak caller (2.2.9.1) with the following parameters: -nomodel -nolambda -keep-dup -call-summits. Peaks from all samples were merged, and peaks overlapping with ENCODE blacklisted regions were filtered out. Peak summits were extended by 150 base pairs (bp) on each side and defined as accessible regions (for footprinting analyses, these peaks were later resized to 1000 bp in width). The fragment counts in peaks and TF scores were calculated using chromVAR (1.24.0). Cell barcodes with less than 30% reads in peaks (FRiP) or 250 unique fragments were removed. The aligned reads were then intersected with peak window regions, producing a matrix of chromatin accessibility counts in peaks (rows) by cells (columns). To examine the cell identity, cisTopic (50 topics) was used for dimension reduction, followed by Louvain clustering. The progenitor populations were sub-clustered to obtain finer cell identity. The data were projected into two-dimensional (2D) space by uniform manifold approximation and projection (UMAP). Seurat V3 (5.0.3) was used to scale the DGE matrix by the total unique molecular identifier (UMI) counts multiplied by the mean number of transcripts, and values were log transformed.

### Generation of BAC Naked DNA Data

Twenty-five chromatin regions were selected based on overlap with a manually selected set of transcription factors and differentiation-related genes. The bacterial artificial chromosome (BAC) clones were cultured in Luria broth (LB) for 14 hours. BAC DNA was extracted using ZR BAC DNA Miniprep Kit following the manufacturer's instructions. The purified DNA was quantified. BAC DNA was tagmented similar to the SHARE-seq ATAC-seq experiment. Briefly, 50 nanograms (ng) of BAC DNA from multiple clones was pooled for tagmentation using the SHARE-seq transposition conditions. The tagmented DNA was purified (e.g., using a polymerase chain reaction (PCR) clean-up kit) and then amplified for seven cycles by PCR. To minimize batch effect, five biological replicates were generated, and all materials were pooled for sequencing. The library was sequenced on a Nova platform (Illumina) using a 100-cycle kit (Read 1: 50 cycles, Index 1: 8 cycles, Index 2: 8 cycles, Read 2: 50 cycles). The sequencing data were processed the same way as the SHARE-seq ATAC-seq data.

### Generation of Human Genomic DNA Data

Human genomic DNA was obtained. The genomic DNA was digested with the restriction enzyme SbfI-HF. For each of two replicates, 25 g of DNA was digested with 200 units of SbfI-HF in a 500 μL reaction at 37° C. overnight. The digested DNA was run on a 1% agarose gel, and fragments corresponding to 2-2.5 kb, 2.5-3 kb, 3-4 kb, and 4-5 kb were excised. All fragments from replicate 1 and the 3-4 kb fragment from replicate 2 were purified using a gel extraction kit. The purified products were concentrated. Finally, tagmentation and library preparation were performed following the above protocol for BAC DNA tagmentation. The same ratio of DNA mass to Tn5 was used when a lower amount of DNA was recovered after size selection.

### In Vitro Footprinting

BAC DNA (e.g., 50 ng) was incubated with recombinant c-MYC/MAX or CEBPA, tagmentation buffer (20 millimolar (mM) Tris, 10 mM MgCl₂, and 20% dimethylformamide (DMF)), and water in a 40 μL volume at room temperature for 1 hour. Then, a master mix comprising 0.15 μL of pre-assembled Tn5, 4.85 μL of dilution buffer (50 mM Tris, 100 mM NaCl, 0.1 mM ethylenediaminetetraacetic acid (EDTA), 1 mM dithiothreitol (DTT), 0.1% NP-40 detergent, and 50% glycerol), and 5 μL of tagmentation buffer was added to the samples for tagmentation in a 50 μL final volume (final TF concentration of 0 nM, 50 nM and 100 nM) for 30 minutes at 37° C. Tagmented DNA was purified using a PCR clean up kit and subsequently amplified for five cycles by PCR. Samples were then pooled and sequenced on a Nova platform (Illumina). Sequencing data were processed in the same way as the bulk ATAC-seq data.

### Aging Multi-Ome Experiment

C57BL/6 mice were housed at a density of 2-5 mice per cage in standard ventilated racks and provided food and water ad libitum in a pathogen-specific free facility accredited by the Association and Accreditation of Laboratory Animal Committee (AALAC). Mouse cages contained Anderson's Bed-o 'Cob bedding, two nestlets (two-by-two inch compressed cotton squares), and a red mouse hut. For hematopoietic stem cell (HSC) isolation and flow cytometry, cells from the bone marrow of long bones (two femurs and two tibias per mouse) from young (n=10; 11 weeks old) and aged (n=5; 24 months old) male C57BL/6 mice were flushed with a 21-gauge needle into staining media (Hanks' Balanced Salt Solution (HBSS)/2% fetal bovine serum), pelleted, and resuspended in ammonium chloride-potassium bicarbonate (ACK) lysis buffer for 5 minutes on ice. The number of mice was determined by the anticipated cell yield and input needs for a single-cell assay; cells from mice were pooled so no blinding or randomization was performed. Cells were then washed with staining media, filtered through a 40 millimeter (mm) cell strainer, pelleted, and incubated with the following cocktail of rat anti-mouse, biotin-conjugated lineage antibodies on ice for 30 minutes: CD3 clone C145-2c11 (1:100 dilution), CD4 clone GK15 (1:400 dilution), CD5 clone 53-7.3 (1:400 dilution), CD8 clone 53-6.7 (1:400 dilution), CD19 clone 6D5 (1:400 dilution), B220 clone RA3-6B2 (1:200 dilution), GR1 (Ly6-G/Ly6-C) clone RB6-8C5 (1:400 dilution), Mac1/CD11b clone M1/70 (1:800 dilution), and Terr119 clone TERR-119 (1:100 dilution). Cells were then washed in staining media, with a small aliquot reserved for each sample to serve as a non-depleted control, and lineage depleted using sheep anti-rat magnetic beads (e.g., Dynabeads) on a magnet. Cells were washed, pelleted, and incubated with the following cocktail of anti-mouse antibodies on ice for 45 minutes to identify hematopoietic stem cells: Pacific Orange streptavidin, phycoerythrin (PE)/Cy7 Sca1(Ly-6a/E) clone D7 (1:200 dilution), APC cKit clone 2B8 (1:200 dilution), fluorescein isothiocyanate (FITC) CD48 clone HM48-1 (1:200 dilution), and PE CD150 clone Tc15-12F12.2 (1:200 dilution). Following incubation, cells were washed and resuspended in staining media, and 7-aminoactinomycin D (7-AAD; 1:50 dilution) was added immediately prior to flow cytometry. Cell sorting of HSCs (Live Lin− Scal+ cKit+ CD48− CD150+) was performed. Cells within the same age group were sorted into the same tube for later sequencing. Data analysis was performed using BD FACS Diva (8.0.2) and FlowJo (10.8.2) software. Data processing was performed using Cell Ranger ARC 2.0.0.

After sorting, nuclei were isolated following 10× Genomics' “Low Cell Input Nuclei Isolation” protocol, which is described in the CG000365 User Guide. Nuclei were then processed using the Chromium Single Cell Multiome ATAC+Gene Expression kit, following the manufacturer's instructions, to obtain between 2,000 and 10,000 cells per sample. Libraries were sequenced on an Illumina Nextseq system using the following sequencing formats: read 1—28, i7 index—10, i5 index—10, Read 2-44 (scRNA-seq), Read 1-30, i7 index—8, i5 index—24, Read 2-30 (scATAC-seq). Data processing was performed using the CellRanger ARC 2.0.0 software from 10× Genomics.

### Tn5 Sequence Bias Modeling

### Getting Tn5 Insertion Counts

The ends of the fragments files were shifted by +4/−4 to obtain the center of the 9 bp staggered end created by Tn5 transposition. The number of insertions at each single base-pair position within each cCRE from each sample was then quantified and stored in a sample-by-cCRE-by-position 3D tensor for fast data retrieval.

### Data Preprocessing

The model took local DNA sequence context as an input and predicted single base-pair resolution Tn5 bias. To this end, the +/−50 bp DNA sequence surrounding each position of interest was encoded by one-hot encoding into a 101-by-4 matrix and used as the model input. For the prediction target, local relative Tn5 bias was used as the target value. More specifically, the raw Tn5 insertion count at each position was divided by the average Tn5 insertion count within a +/−50 bp window. Positions with low local coverage (<20 insertions per bp) were removed to improve the quality of training data. To facilitate model training, the resulting observed Tn5 bias values were log 10-transformed and rescaled. For dataset partition, all the BACs were randomly split into 80%, 10%, and 10% for training, validation, and test sets, respectively. In other words, all data originating from the same BAC belonged to the same partition. This was to prevent overlapping local sequence contexts ending up in both training and testing datasets, which might lead to overestimation of performance. To provide equal coverage of examples with different bias levels, all training examples were binned into 5 bins based on their Tn5 bias values, and each bin was up-sampled so that all bins ended up with the same number of examples. Additionally, given the symmetric nature of Tn5 insertion, reverse complement sequences of the training examples were generated as data augmentation. The original and reverse complement data were combined, shuffled, and then used for model training.

### Model Architecture

The convolutional network included three convolution and max-pooling layers and two fully connected layers. Each convolution and max-pooling layer performed a convolution, a ReLU nonlinear activation, and max pooling sequentially. Thirty-two filters of width five were used for each layer, along with “same” padding mode and stride size of one. The two following fully connected layers had output dimensions of 32 and 1, respectively. ReLU activation was used by the first fully connected layer, and linear activation was used by the second layer (i.e., the final output layer).

### Model Training and Evaluation

The model was trained on the training set, and hyperparameters were optimized based on performance on the validation set. Final performance of the frozen model was evaluated on the test set. The model was implemented using Keras, trained with mean square error as a loss function, and optimized using the Adam optimizer with default parameters. Training was performed with a batch size of 64 and early stopping based on model loss on the validation set.

Benchmarking with Other Tn5 Bias Models

Methods including k-mer models (k=3, 5, 7) and PWM methods (single nucleotide and dinucleotide) were included in benchmarking. For k-mer methods, the foreground and background frequencies for all possible k-mer sequences were quantified. The foreground frequency/background frequency ratio was used as the estimated Tn5 bias for the corresponding k-mer. For single nucleotide PWM, foreground and background base frequencies within a +/−10 bp window (total length=21) were calculated, and the PWM of Tn5 insertion was computed. Dinucleotide PWM scores were calculated using TOBIAS with default settings. Custom ChromBPNet bias models were trained on inaccessible chromatin regions for each dataset to represent the Tn5 sequence bias and achieve the highest-quality models. Accordingly, ChromBPNet was trained on HepG2 and K562 ATAC-seq data to evaluate its performance in its recommended setting.

### Genome-Wide Tn5 Bias Reference Tracks

Sequences of reference genomes for Homo sapiens (hg38), Mus musculus (mm10), Drosophila melanogaster (dm6), Saccharomyces cerevisiae (sacCer3), Caenorhabditis elegans (cel1), Danio rerio (danRer11), and Pan troglodytes (panTro6) were downloaded from the UCSC genome browser website. The aforementioned Tn5 bias neural network model was applied to each position in the reference genomes to generate genome-wide Tn5 bias tracks.

### Computing Footprint Scores

To detect DNA-protein interactions at different scales within cCREs, a framework for computing footprint scores for each base pair position in the cCRE was implemented. In short, for each single bp position, a center footprint window and flanking windows on both sides were defined (FIGS. 7A and 7B). Then, the observed ratio of center/(center+flanking) Tn5 insertion counts was calculated. The foreground observed ratio was compared to a background distribution to calculate statistical significance, which was then converted to a footprint score.

### Estimation of Background Dispersion

Given a specific combination of center bias, flanking bias, and local coverage, a certain distribution of center/(center+flanking) insertion ratio was expected when no protein was bound. This was defined as the background distribution. Such background distribution was estimated using BAC naked DNA Tn5 insertion data. To this end, 100,000 positions were randomly sampled from the BAC dataset, and their local coverage (defined as the total insertion number in center and flanking areas), center bias, as well as flanking bias were retrieved. For each sampled position A, 500 nearest neighbor (NN) positions NN1-NN500 were identified in the three-dimensional space of (center bias, flanking bias, local coverage). To ensure each dimension was weighed equally, the values of each dimension were first normalized to zero mean and unit variance. The 500 nearest neighbor observations were considered as background observations with nearly identical bias and coverage, and the center/(center+flanking) ratio of NN1-NN500 formed the background distribution of position A. Therefore, for each of the 100,000 sampled positions, the mean and standard deviation of its background ratio distribution were calculated. This allowed training of a background dispersion model that takes the tuple (center bias, flanking bias, local coverage) as input and predicts the mean and standard deviation of the background distribution efficiently. To ensure the model was exposed to training examples with a wide range of local coverage, the BAC dataset was down-sampled to 50%, 20%, 10%, 5%, and 1% of the original sequencing depth. Finally, a neural network with a single hidden layer (32 nodes, ReLU activation) and linear output layer activation was trained. The dataset was randomly split into 80% training, 10% validation, and 10% test. The model was implemented using Keras and trained on the training dataset with mean squared error loss using the Adam optimizer. Early stopping was determined using loss on the validation set, and performance of the final model was evaluated on the test set. Additionally, separate models were trained for each footprint radius due to the drastic differences in total center or flank bias when footprint radius varies.

### Calculating Footprint Scores

For each position in the cCRE, a center footprint window and flanking windows on both sides were defined. First, the foreground observed center/(center+flanking) ratio of Tn5 insertion counts was calculated. Then, the pre-trained background dispersion model was applied to calculate the mean and standard deviation of its background distribution. Next, a lower-tailed z-test was used to calculate the p-value for footprinting. If the observed ratio was significantly lower than the background distribution, then this position was likely to be bound by a protein. More specifically, to avoid calling footprints at positions where only one flanking side showed higher Tn5 insertion than the center window but not the other, center-versus-left and center-versus-right tests were performed separately, and the larger p-value was kept, as further explained below. The −log 10(p-values) were smoothed by running-max and running-mean smoothing and then used as the final footprint scores.

### Framework for Statistical Testing

For each position in the CRE, a center footprint region and a flanking region were defined (FIG. 10). A footprinting score was then calculated using statistical testing. The test statistic 2 was the ratio of total Tn5 insertions in the footprint region divided by the total Tn5 insertions in the footprint and flanking regions combined, defined as:

$$λ = \frac{\text{?} {x}_{i}}{\text{?} {x}_{i} + \text{?} {x}_{i}}$$

$$\text{?} \text{indicates text missing or illegible when filed}$$

where xᵢ is the number of Tn5 insertions at position i. A_{flank} and A_{footprint} are the sets of position indices in the flanking and footprint regions, respectively. The goal was to estimate the background distribution of 2 when no protein is bound and then compare the observed value of 2 to its background distribution. If the position of interest was protected from Tn5 insertion by a protein, the observed 2 should be significantly lower than the background distribution. Hence, a p-value was calculated to represent the significance of such deviation.

Two tests were performed on each side (i.e., center-vs-left and center-vs-right), and then the less significant p-value was kept as the result. More specifically, λ_{left} and λ_{right} were calculated.

$${λ}_{left} = \frac{\text{?} {x}_{i}}{\text{?} {x}_{i} + \text{?} {x}_{i}} , {λ}_{right} = \frac{\text{?} {x}_{i}}{\text{?} {x}_{i} + \text{?} {x}_{i}}$$

$$\text{?} \text{indicates text missing or illegible when filed}$$

A_{flankL} and A_{flankR} are the sets of position indices in the left and right flanking regions, respectively. The reason behind testing each side was to reduce false positive results. The case where one accessible CRE is flanked by two nucleosomes was considered.

For positions at the edge of the accessible region, Tn5 insertion was low in the center footprint region as well as one of the two flanking regions. High Tn5 insertion was observed in the other flanking region. Comparing the footprint region with both flanks combined could lead to the detection of false positive signals since 2 could still be lower than expected. However, performing two tests on each side and retaining the less significant result resolved such issues.

### Modeling the Background Distribution

BAC DNA Tn5 insertion data was utilized to estimate the background distribution of λ. It was reasoned that for each flanking side, the background distribution of λ should be determined by center and flanking Tn5 bias, as well as the total number of reads in center and flank. Assuming b is the vector of predicted Tn5 bias (i.e., predicted bias at position i is bᵢ), the following was established:

$${b}_{left} = {\sum }_{i ∈ {A}_{flankL}} {b}_{i} , {b}_{center} = {\sum }_{i ∈ {A}_{center}} {b}_{i} , {b}_{left} = {\sum }_{i ∈ {A}_{flankR}} {b}_{i}$$

$${c}_{left} = {\sum }_{i ∈ {A}_{flankL} ⋃ {A}_{center}} {x}_{i} , {c}_{right} = {\sum }_{i ∈ {A}_{flankL} ⋃ {A}_{center}} {x}_{i}$$

where b_{left}, b_{center}, and bright are total biases in the left flanking, center footprint, and right flanking regions, respectively; and c_{left} and c_{right} are coverage for the left and right side testing, respectively. This enabled the following distributions to be modeled:

$${λ}_{left} ∼ {F}_{left} ( {b}_{left} , {b}_{center} , {c}_{left} )$$

$${λ}_{right} ∼ {F}_{right} ( {b}_{right} , {b}_{center} , {c}_{right} )$$

where F_{left} and F_{right} are the distribution functions for λ_{left} and λ_{right}, respectively.

The most straightforward approach would be to use the BAC naked DNA data as a lookup table. To compute the footprint score for a specific position (referred to as “foreground”) in an ATAC-Seq dataset, the center bias, flanking bias, and coverage for the foreground observation were computed. Next, the BAC naked DNA data was searched to find the k=500 nearest neighbor (KNN) observations in the (center bias, flanking bias, coverage) 3-dimensional space. The λ for these background observations was computed and denoted as λ_{bg}. The distribution of λ_{bg} was then used as the background distribution for the foreground observed ratio λ_{obs}, and a p-value was computed using z-test. To ensure the KNN matching weighed the three features equally, coverage values were first log 10-transformed. and then all three features were standardized before KNN matching. Additionally, to cover a wide range of coverage values, the BAC dataset was down-sampled to 100%, 50%, 20%, 10%, 5%, 2%, and 1% and then pooled before KNN matching.

In practice, performing KNN matching for each foreground observation is extremely time-consuming. Therefore, a neural network dispersion model was instead trained to learn the following relationship:

$$( {μ}_{left} , {SD}_{left} , {μ}_{right} , {SD}_{right} ) ∼ F ( {b}_{left} , {b}_{right} , {b}_{center} , {c}_{left} , {c}_{right} )$$

A number of observations (e.g., 100,000) were randomly sampled from the BAC dataset. For each of them, 500 nearest neighbor observations in the BAC dataset were matched, and the distribution of λ_{b}g was computed. Then, these 100,000 observations along with their background distribution were used to train the dispersion model.

### Aggregate Footprinting

To calculate aggregate footprints, Tn5 insertions surrounding TF or nucleosome binding sites across the genome were first aggregated and then used to calculate footprint scores. For TFs, sites with a matched TF motif using motifmatchr (p.cutoff=1e-5) and overlapping with a ChIP-seq peak of the corresponding TF were selected. For motif matches on the reverse strand, the Tn5 insertion profile surrounding the motif was inverted so the insertions for different sites were aligned in the same direction.

### Footprint-to-TF Prediction

While seq2PRINT was chosen as the primary TF binding predictor, this light-weight Footprint-to-TF prediction model is still provided for its speed advantages. For comparison between Footprint-to-TF prediction and seq2PRINT-based TF binding prediction, see the “Multiple Methods to Predict TF Binding from Multi-Scale Footprinting” section below.

### Input Data

To predict the landscape of TF binding, a binary classifier was trained that predicts whether any TF motif site is bound by the corresponding TF. Motif sites were identified by the matchMotifs function in the motifmatchr package. All sites with a matching p-value below 5e-5 were kept. For any TF motif site, multi-scale (20 bp, 40 bp, 60 bp, 100 bp, 160 bp, 200 bp in diameter) footprints within a +/−100 bp local area centered around the motif and a motif match score were used as input to the model. The motif match score returned by the matchMotifs function was quantile-transformed to a uniform distribution. As a result, by combining the 201-dimensional footprint vectors from six different scales with a single motif match score, a 1207-dimensional vector was obtained as the final model input. The first of the 1206 dimensions of footprint scores were standardized individually to zero mean and unit variance. For the prediction target, a label of 1 was assigned to all sites overlapping with a ChIP peak of the same TF, and a label of 0 to sites not overlapping with ChIP. Some TFs were found to have a very low percentage of motif sites overlapping with ChIP (≤10%), potentially due to low quality of the motif or the ChIP dataset and were removed from model training and testing. Additional negative examples as well as reverse-complement examples were added for data augmentation. TFs with >10% bound motifs were kept.

For data partition, HepG2 data were used as training data, and GM12878 data (previously published in the original SHARE-seq paper) were used as validation data. After fixing model hyperparameters, HepG2 and GM12878 data were combined into a larger training dataset to train a final footprint-to-TF model. The final model was tested on K562 single cell ATAC data as well as three cell types (naive B cells, CD14 monocytes, and late-erythroid cells) in the human BMMC SHARE-seq dataset.

### Model Architecture and Training

The TF binding prediction model is a neural network model with two hidden layers (128+32 nodes). ReLU activation was used by both hidden layers, and sigmoid activation was used by the final output layer. The model was implemented using Keras. The model was trained on the training dataset with a batch size of 128 using the Adam optimizer. Binary cross entropy was used as the loss function. Early stopping was used based on model loss on the validation set.

ChIP Validation and Benchmarking with Previous Methods

To evaluate model performance, ChIP-seq was used as ground truth data, and predicted binding events were validated. HepG2 and GM12878 data were downloaded from Unibind for model training. ChIP-seq for BMMC cell types were downloaded from cistromeDB. For benchmarking with previous methods and to ensure only high quality TF binding sites were included, K562 ChIP-based TF binding data were downloaded from Unibind. For cistromeDB datasets, QC filters as specified on the cistromeDB website were applied. More specifically, the following filters were included: FRiP≥0.01, FastQC≥0.25, uniquely mapped ratio ≥0.6, peaks with fold change above 10 and ≥500, peaks union DHS ratio ≥0.7, and PBC≥0.8. Datasets with the following cell type labels were included: “Monocyte,” “B Lymphocyte,” “Erythroid cell,” “Erythroid Progenitor Cell,” and “Erythroid progenitor.” If there was more than one dataset for the same TF, the intersection of all datasets for the same TF was kept as the final list of high confidence binding sites for model training.

The K562 datasets from Unibind were used for benchmarking with previous methods, including DNase I footprinting, TOBIAS, and sequence attribution scores obtained from ChromBPNet. In short, the same ATAC-seq data were used as input to all ATAC footprinting methods and ChromBPNet, while DNase I footprinting results in K562 were obtained from the ENCODE datasets ENCLB253REF, ENCLB843GMH, and ENCLB096YUZ. To guarantee fair comparison, the same set of motif match positions as used previously were used as candidate binding sites, and the predicted scores of each method were mapped to these sites for comparison. For TFs with multiple Unibind datasets, their intersection was used for benchmarking. Then, for each method, the candidate sites were ranked by predicted binding score, and precision of prediction was evaluated using the top 10% of sites. Visualization of predicted and ground truth binding sites was done with the Gviz package (1.46.1). Furthermore, to evaluate the false positive rate of each model, all three ATAC-based models were also tested on the BAC naked DNA data. The same data were used as input to each model, and the number of predicted binding events were used to represent the false positive predictions.

### Footprint-to-Nucleosome Prediction

### Input Data

To predict nucleosome occupancy, a regression neural network model was trained. For any genomic position, multi-scale (20 bp, 40 bp, 60 bp, 100 bp, 160 bp in diameter) footprints within a +/−100 bp local area were used as input to the model. The 200 bp scale was not included to prevent the model from learning co-occupancy of adjacent nucleosomes. To train this model, previously published chemically mapped nucleosome occupancy data in S. cerevisiae was used as training labels, and multi-scale footprints were computed using previously published S. cerevisiae ATAC-seq data as training input. Observations in regions with local ATAC-seq coverage >10 were kept, and the 5% and 95% percentile of nucleosome occupancy values were rescaled to 0 and 1, respectively. For data partition, all data were randomly split by chromosomes into training (chrVII, chrXI, chrIX, chrI, chrV, chrX, chrVIII, chrXII), validation (chrIV, chrIl), and test (chrVI, chrXVI, chrXIII, chrIII, chrXIV, chrXV) sets.

### Model Architecture and Training

The nucleosome prediction model is a neural network model with two hidden layers (64+16 nodes). ReLU activation was used by both hidden layers, and linear activation was used by the final output layer. The model was implemented using Keras. The model was trained on the training dataset with a batch size of 128 using the Adam optimizer. Mean squared error (MSE) was used as the loss function. Early stopping was used based on model loss on the validation set.

### Performance Evaluation

Model performance was evaluated using data of the test yeast chromosomes mentioned above. In total, 859 regions with length of 5 kb each on the test yeast chromosomes (chrVI, chrXVI, chrXIII, chrIII, chrXIV, chrXV) were used for testing. Summits of predicted nucleosome signal and ground truth nucleosome occupancy were detected as predicted and observed nucleosomes, respectively. Precision was calculated as the percentage of predicted nucleosomes having a ground truth nucleosome within a certain distance cutoff (50 or 75 bp in this study). Recall was calculated as the percentage of ground truth nucleosomes having a predicted nucleosome within the same distance cutoff.

### Seq2PRINT

### Model Architecture and Training

The seq2PRINT model is a convolutional neural network that takes one-hot encoded DNA sequences (a DNA sequence of length L encoded into an L×4 matrix, where each row has one element set to 1 representing the specific nucleotide) as input and predicts the corresponding multi-scale footprints at base-pair resolution. The architecture is depicted in FIG. 16. To facilitate articulating the architecture, the seq2PRINT model was divided into three parts. While the architecture of seq2PRINT can be flexible depending on the available computational resources and the depths and scale of the training data, for all results in this work, the model was built and hyperparameters were chosen as follows.

The first convolutional layer included 1,024 filters of width 21 bp with Gaussian Error Linear Units (GELUs) activation, aiming to capture informative sequence patterns from the input DNA sequences (i.e., sequence motifs). The output was subsequently passed to eight layers of convolutional blocks with residual connection. Each convolutional block included one grouped dilated convolutional layer (n_filters=1,024, width=3, groups=8, dilation=2\hat{ }i, i=1, . . . , 8) followed by a position-wise feed forward layer (implemented as a convolutional layer with n_filters=1,024, width=1). Batch normalization layers with GELU activations were inserted between these convolutional blocks. The increased dilation rate resulted in an expanding receptive field for the neural network, capturing the relationship of the sequence patterns and their context. With eight layers of the convolutional block, seq2PRINT had a receptive field of 420 bp for each cCRE. The use of grouped convolutional layers enabled the construction of wider models that captured richer information with reduced parameters, providing a regularization effect and reduced computational complexity. Finally, the output of the stacks of convolutional blocks was passed to the output layers.

In this Example Application, two output layers were designed: a multi-scale footprint layer (a convolutional layer of filter width 1) that outputs the multi-scale footprints, and an accessibility layer (a global average pooling layer followed by a fully connected layer) to predict the number of Tn5 insertions in a specific cCRE.

To facilitate the training of the seq2PRINT model, batch-efficient multi-scale footprint calculation on GPU was implemented, which follows the same mathematical models as the described footprint calculation with the only difference being that it outputs the z-statistics instead of the p-value calculated from the z-test.

During training, the model weights were updated to minimize the following loss function:

$$Loss = {Loss}_{footprint} + {Loss}_{accessibility}$$

$${Loss}_{footprint} = MSE ( {footprint}_{PRINT} , {footprint}_{pred} )$$

$${Loss}_{accessibility} = MSE ( log ( 1 + {n}_{obs} ) , log ( 1 + {n}_{pred} )$$

where MSE represents the mean-squared error MSE(x,y)=Σ(x−y)², footprint_{PRINT} and footprint_{pred} represent the multi-scale footprints calculated by the PRINT framework and the seq2PRINT model, respectively, and n_{obs} and n_{pred} represent the observed and predicted Tn5 insertions in this region, respectively.

Notably, the gradient back-propagation for the accessibility layer is broken before the convolutional blocks. In other words, the sequence patterns and relationships among them learned by the preceding layers of the seq2PRINT model are driven solely by the multi-scale footprint objective. The accessibility output layer and corresponding loss function only reweight these learned sequence features for interpretation purposes, as further described below. This design alleviates the need to choose weights between the footprint loss and accessibility loss and also makes seq2PRINT a footprint-driven sequence model, differentiating it from previous accessibility-driven models (e.g., Basset).

The seq2PRINT model was optimized with the Adam optimizer with a learning rate of 3e-4 and employs exponential moving averages to stabilize and improve the convergence of the model. In this study, chromosome based 5-fold cross validation was used, and the outputs across folds were averaged to use as the final predictions.

### Deriving Sequence Attribution Scores

The DeepLIFT method was used to calculate the sequence attribution scores, which represents how each base pair in a given input DNA sequence contributes to a specific scalar output from the trained seq2PRINT model. The output of the accessibility layer is a scalar for each region, making it naturally suitable as the target for DeepLIFT to calculate the attribution scores. However, the multi-scale footprint layer is not a scalar but a matrix of size L times the number of scales. Therefore, two strategies were designed to summarize the output footprints into a scalar value.

Both strategies involve converting the predicted z-statistics to log p-value footprint scores as the PRINT framework. The first strategy involves manual inspection, as demonstrated in FIG. 17, where the region×scale of interest from the observed and predicted multi-scale footprints is located, the footprint scores within the region are summed, and the sequence attribution score corresponding to it is calculated. The second strategy sums the footprint scores within the whole peak region and is more suitable for genome-wide calculations. Without further specification, the sequence attribution scores calculated from the accessibility layer are referred to as the count sequence attribution scores, and the ones calculated from the footprint layer as the footprint sequence attribution scores.

For the results described herein, 20 dinucleotide-shuffled input sequences were used as the reference sequences for the DeepLIFT algorithm. The implementation of DeepLIFT algorithm from the DeepSHAP package was adapted to accommodate the custom nonlinear functions used in this framework.

### De Novo Motif Identification Based on Sequence Attribution Scores

TF-MoDISco (tfmodisco-lite v2.2.1) was utilized to infer de novo motifs based on the sequence attribution scores. Briefly, TF-MoDISco identifies local regions in input sequences with high sequence attribution scores (seqlets), then aligns and clusters similar seqlets into groups of de novo motifs. The number of seglets was set as 1,000,000, and the rest parameters were set as default. The de novo discovered motifs were assigned to known motifs using TomTom (meme suite v5.5.7). To infer the matching of de novo motifs at CREs, the software finemo (commit number 830d7f3) was used which takes both the de novo motifs and sequence attribution scores.

### Fine-Tuned TF Binding Prediction Using Attribution Scores

The calculated sequence attribution scores highlight TF binding sites affecting footprints and accessibility. A binary classifier was thus trained that is similar to the footprint-to-TF model, but instead of multi-scale footprints, it took the count and footprint sequence attribution scores.

The training and validation TF binding sites remained the same as the previous footprint-to-TF model. For each motif site, the features included the count and footprint sequence attribution scores within a +/−100 bp area centered around the motif, the motif matching score, and the Pearson correlation between the motif of interest and the sequence attribution score at the motif matching site. In total, this produced a 405-dimensional vector.

The fine-tuned TF binding model included three hidden layers (256, 128, 64 neurons), with GELU activations and 0.25 dropout rates in between. The model was trained with the Adam optimizer, with binary cross entropy as the objective function.

This fine-tuned TF binding model was the final TF binding prediction model used due to its superior performance. A comparison between Footprint-to-TF prediction and seq2PRINT-based TF binding prediction can be found in the “Multiple Methods to Predict TF Binding from Multi-Scale Footprinting” section below.

LoRA Enables Efficient Sequence Modeling for scATAC-Seq Data

To make the sequence model much more scalable on scATAC-seq with diverse cell types or cell states, the LoRA (low rank adaptation) technique was employed for parameter efficient finetuning of neural network models. Specifically, for a given scATAC-seq data, a seq2PRINT model (subsequently referred to as the pretrained model) was first trained by aggregating Tn5 insertions over all cells in the dataset. Next, for each pseudo-bulk aggregating cells over specific cell states, the LoRA fine tuning technique was used to derive a pseudo-bulk specific seq2PRINT model. The fine tuning process is described as follows.

For any neural network layer parameterized by W₀∈ [symbol] ^{d}^{ₚ}, the LoRA model learned an updating parameter ΔW∈ [symbol] ^{d}^{ₚ}, where dₚ is the number of learnable parameters of the layer, and the sum of these two parameters W=W₀+ΔW was used as the parameter for the fine-tuned neural network model. For the residual grouped convolutional layer used in seq2PRINT,

$${d}_{p} = \frac{n\_filter}{group} \times n\_filter \times kernel\_size$$

was used.

If the model is fine-tuned individually for n pseudobulks of interest, this results in total of n×dₚ parameters to be learned.

Motivated by the LoRA model, a low-rank decomposition of this updating parameter was instead learned, guided by the intrinsic low-rank of cell states that the cell embeddings capture. Specifically, two sets of weights

$$A ∈ {ℝ}^{{d}_{e} \times r + r \times \frac{n \_ filter}{group}}$$

and B∈ [symbol] ^{d}^{ₑ}^{×r+r×n_filter×kernal_size} were learned, where dₚ, is the dimension of the single cell embeddings, and r is the hyperparameter referred to as the rank. This design reduces the amount of parameters to learn from

$$n \times {d}_{p} to r \times ( {d}_{p}^{*} + {d}_{e} ) ,$$

where

$${d}_{p}^{*} = \frac{n\_filter}{group} + n\_filter \times kernel\_size < \frac{n\_filter}{group} \times n\_filter \times kernel\_size .$$

In this study, r=32 was chosen, which is much smaller than the number of pseudo-bulks studied.

### Predicted Impact of TF Motifs on Footprints

To probe the relationships between multi-scale footprints and DNA sequence the seq2PRINT model learns, the marginalized prediction from the seq2PRINT was generated given a known or de novo discovered motif. For a de novo discovered motif of interest, the consensus sequence was first identified by taking the nucleotide with highest probability at each position. Then, 25,000 CREs were randomly selected from the dataset. The consensus sequence was planted at the center, and the model predictions around the sampled CREs were averaged. For a known motif, its motif matching positions in cCREs were gathered, the motif matching positions were scrambled and the model predictions were averaged. In both approaches, the marginalized prediction was calculated as the difference when the given motif is present and absent.

### Multi-Scale Footprinting

There were two main motivations behind using multi-scale footprinting for object detection. The first motivation was that some DNA-binding factors did not leave footprints on their own, potentially due to weak or transient binding. Therefore, the binding of such factors could only be inferred through the binding and positioning of nearby objects such as nucleosomes. In these cases, using only footprints detected at the scale corresponding to the size of the object itself led to false negative results (e.g., only using the 40 bp scale footprint to detect YY1 binding).

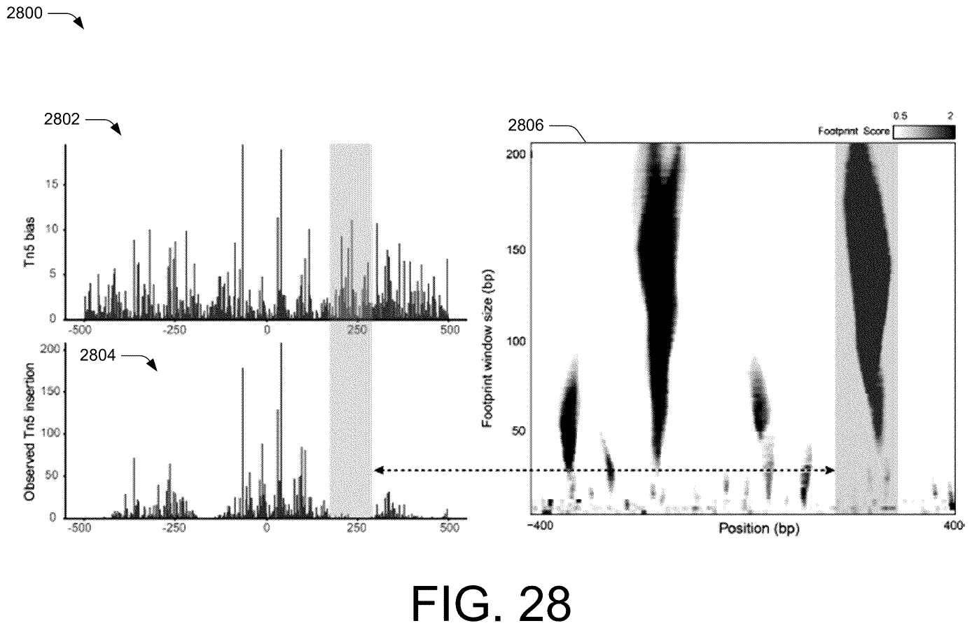

The second motivation was to filter signal bleed through across scales. It was observed that the footprint signal of an object could bleed through into lower scales, potentially due to non-linear impact on Tn5 insertion by object binding. For instance, the example depicted in FIG. 28 was examined.

FIG. 28 shows an example 2800 of Tn5 bias and observed Tn5 insertion for a genomic region that is footprinted. A Tn5 bias plot 2802 shows insertion bias measurements across a genomic region, with peaks of varying heights indicating different levels of Tn5 insertion bias. A footprint score plot 2804 presents footprint scores across the same genomic coordinates. A footprint window size plot 2806 shows footprint patterns at different scales, with darker regions indicating stronger footprint signals and a scale bar ranging from 0.5 to 2. Highlighted boxes represent the same genomic region, which shows footprint signals from around 40 bp up to 200 bp scales, representing a bound nucleosome. Although the nucleosome-bound region was around 140 bp in diameter, the footprint signal extends to smaller spatial scales. This occurs because nucleosome binding does not affect Tn5 insertion equally at all positions. Positions with very high Tn5 bias, shown as “spikes” in the Tn5 bias plot 2802, are affected most dramatically. In the highlighted region, several positions have Tn5 bias spikes, which the model expects to have a high center/(center+flank) ratio of Tn5 insertion. However, in the observed insertion track, due to nucleosome binding, Tn5 insertion at these locations is suppressed to a low baseline level, similar to flanking regions. Consequently, the observed center/(center+flank) ratio is lower than expected, resulting in significant footprint signals at such positions. The example 2800 demonstrates how the unequal impact of bound objects on Tn5 insertion affects the footprint analysis performed by the DNA-protein interaction analysis module 124. In other words, if insertion at all base pairs in the bound regions were reduced by the same scaling factor, including positions with Tn5 bias spikes, then most of the bleed-through would no longer appear.

As a result, if TF binding was detected only by examining footprint signals at the scale matching the size of TFs, such bleed-through resulted in false positive signals. The bleed-through effect was theoretically not specific to this footprinting method. For any method that defined a footprint window and calculated deviation of observed Tn5 insertion in the footprint window from the expected level, this could be a potential source of false positive signal. Positions with Tn5 bias spikes but bound by nucleosomes tended to have high expected but low observed cutting and could show up as a false positive footprint at the scale of TFs.

Given the above, it was realized that trying to detect objects using footprint scores at a single scale and a single location was under-powered. Instead, footprint signals across scales and positions were leveraged for accurate object detection.

Multiple Methods to Predict TF Binding from Multi-Scale Footprinting

Multiple methods to predict TF binding were explored due to the differing strengths of each method. Overall, they fell into two categories: (1) Footprint-to-TF prediction, which used local multi-scale footprints as model input, and predicted whether a motif site was bound by the TF or not; and (2) seq2PRINT, which first trained a model that used DNA sequence to predict multiscale footprints. The sequence attribution scores derived from the model were then used to predict TF binding.

In terms of predictive power, seq2PRINT was superior in both accuracy and resolution (as it could score each single base pair on their contribution to footprint prediction). Hence, seq2PRINT is recommended when there are adequate computational resources. However, seq2PRINT is more resource-intensive due to training a new convolutional neural network for each new dataset and computing sequence attribution scores. Footprint-to-TF prediction used a single light-weight model for all datasets and ran significantly faster at the cost of moderate precision loss. Therefore, the footprint-to-TF model and/or seq2PRINT can be used depending on use case.

### Multi-Scale Footprinting Versus V-Plot Analysis

V-plot analysis is an alternative method for representing DNA-protein interactions across spatial scales that had been applied to DNAse-, MNase-, and ATAC-seq. However, there are differences between multi-scale footprinting and V-plot methods. V-plot methods mostly examine the position-fragment size relationship and often did not account for sequence biases. For assays such as ATAC-seq where sequence bias is very strong, the V-plot is heavily confounded, especially for the detection of TFs.

Even with accurate bias correction, background dispersion of Tn5 insertion is modeled due to random noise. When data is sparse and noisy such as in pseudo-bulked scATAC-seq data, it is important to distinguish background noise and events that are statistically significant, particularly when evaluating single loci (as opposed to aggregating across motifs).

Multi-scale footprinting provides a feature map that is amenable to downstream statistical or deep learning analysis, as it separates the footprints of different objects along the x and y axes. By comparison, in V-plots, the footprints of nearby objects could be convoluted by overlapping V patterns. Discrete footprints enable analyses where seq2PRINT attribution scores for individual footprints are examined. It also allows sub-sampling of the multi-scale pattern at representative scales to enable signal filtering and reduce the computational load for machine learning tasks.

A particular strength of V-plot analysis is the use of fragment size to further define regions protected from nuclease/transposase activity. During the initial development of PRINT, using this feature was explored, and it did not enhance the accuracy of footprint detection. However, it can be used as a parameter in seq2PRINT-like deep learning.

### Tracking TF Binding Dynamics Across Human Hematopoiesis

### Generation of Pseudo-Bulks

Single cells in the human BMMC dataset were first embedded into lower dimensional space using cisTopic and then grouped into 1000 pseudo-bulks based on their spatial proximity in the cisTopic space. More specifically, 1000 cells were first sampled as pseudo-bulk centers, and then k-nearest neighbors (KNN, k=5000) of each center cell in the cisTopic space were identified as other members of the same pseudo-bulk. It was reasoned that sampling center cells with low local connectivity can help increase coverage of the state space by preventing over-sampling of densely connected local neighborhoods. Therefore, 10,000 scaffold cells were first randomly sampled and used to construct a KNN graph (k=10). Then, the 1000 cells with the lowest in-degree in the KNN graph were selected as pseudo-bulk centers.

### Computing Pseudo-Time

Pseudo-time along human hematopoietic lineages was computed using the Palantir package (1.0.0). To reduce computing time, 100,000 cells were randomly sampled from the human BMMC dataset as scaffold cells. The cisTopic embedding of the scaffold cells as well as pseudo-bulk center cells were used as input to Palantir.

### Footprinting Repressor TFs

As BHLHE40 was extensively studied for its role in T cells, BHLHE40 footprinting results were obtained using CD8+ T cells from the human bone marrow dataset.

Tracking the Dynamics of TF Binding in Erythroid and Lymphoid Cell cCREs

For erythroid and lymphoid development trajectories, relevant genes were first identified by selecting genes with a correlation between RNA level and pseudo-time >0.5 respectively. Then, CREs that were within +/−50 kb from the transcription start sites (TSSs) of related genes and the correlation between CRE accessibility and pseudo-time >0.5 were filtered. For all CREs, candidate TF binding sites activated during the development were located by keeping sites where the correlation between seq2PRINT TF binding score and pseudo-time >0. The remaining TF binding sites were used for dynamic quantifications. To quantify the inferred timing of binding, the TF binding score of each binding site was rescaled to [0,1], and the area-under-curve (AUC) was calculated. Higher AUC values represent earlier rising of binding signals and vice versa.

Principal Component Analysis Measures the Complexity of TF Binding Patterns at Each cCRE

To reveal the complexity of the TF binding patterns within each cCRE across diverse cell population in the human BMMC dataset, a principal component analysis (PCA)-based method was used. To reduce the computational complexity, the 1000 LoRA fine-tuned seq2PRINT models (corresponding to 1000 pseudo-bulks) were first collapsed into 20 models that correspond to 20 cell types. Model weights of pseudo-bulks corresponding to the same weight were averaged during this process. Then, cCRE-wide TF binding scores for these 20 cell types were generated. Each cCRE was tiled with 10 bp windows, and the TF binding scores within each window were averaged. PCA was then used as a dimension reduction method on this 20×10 bp window matrix for each CRE, and the minimum number of PCs that explains 98% of the variance was used to quantify the complexity of the TF binding patterns.

Characterizing Age-Related cCRE Reorganizations

### Data Preprocessing

Cells with a fraction of reads in peaks (FRIP)<0.3 and a depth<300 were removed. Additionally, ArchR was used to calculate doublet scores for each single cell, and cells with the top 5% doublet scores were removed. The remaining cells were then processed with the Seurat V3 package (5.0.3). Cells were embedded into lower dimensional space using latent semantic indexing (LSI) and then clustered. Seurat clusters corresponding to HSCs were selected for pseudo-bulking and downstream differential testing. Cells with the “LinNeg” FACS sort label were excluded for HSC-specific analyses. To identify representative cell states, SEACells was used to identify 100 representative cell states across HSCs. The representative cells were used as centers to form pseudo-bulks. Each pseudo-bulk was generated by serially including nearest neighbor cells from the center cell in an order of increasing distance until a total of 5 million fragments was reached.

### Defining HSC Subpopulations

The single cell RNA expression data obtained from 10× multi-ome was first filtered by removing the top 5% most highly expressed genes, as well as ribosomal genes and mitochondrial genes. Cells with lower than 100 RNA counts were removed. SCTransform (0.4.1) was then run with 5000 variable features to normalize the data. The normalized values were used as input to Spectra. Spectra was next run initialized with default HSC and global pathways, and additional gene sets were obtained from published literature. Gene sets with fewer than five genes covered were removed. The expression of each Spectra program in each pseudo-bulk was scored. To this end, DESeq2 was used to normalize the pseudo-bulk-by-gene RNA count matrix, and the values were rescaled per-gene. For any specific Spectra program, 100 background programs including genes with matched overall expression levels were generated. The average gene expression in the Spectra program-of-interest was then compared to the average gene expression in sampled background programs to derive a z-score. Finally, the pseudo-bulk-by-program z-score matrix was used to cluster pseudo-bulks into HSC subpopulations.

### Differential Testing

Differential RNA and cCRE accessibility testing was performed using DESeq2 (1.42.1). For each pseudo-bulk, total RNA read counts for each gene and Tn5 insertion counts in each peak (resized to 1 kb) in each pseudo-bulk were quantified, and DESeq2 was used to identify significant differential genes and cCREs with age as the covariate.

### Predicting Ets/Runx Dimer Structures Using Alphafold3

For each de novo identified motif representing the Ets/Runx composite motif, the consensus DNA sequence was generated. The same segments of the amino acid sequences of Ets and Runx as those in Protein Database (PDB): 4L0Z (Runx: 54-212, Ets: 332-432) were then input into Alphafold3 along with the consensus DNA sequence and its reverse complements. For the structure corresponding to the validated dimer structure, the AF3 predicted structure was aligned to the PDB structure using PyMol (2.6), and the root mean square deviation (RMSD) was calculated. All structures were visualized using ChimeraX v1.8. Solvent-inaccessible bases were identified using ChimeraX interface function with default parameters.

### HSC Subtypes

For the mouse HSC aging analysis, it was examined whether the previously reported HSC subtypes could be recapitulated by the presently developed dataset. Representative cell states were first defined using the ATAC data. To do this, SEACells was used, which uses a graph embedding to define maximally distinctive states. SEACells was designed to achieve a good balance between signal detection and cell state resolution. One hundred meta-cells were chosen as it sampled the diversity of the data set without creating overwhelming computational complexity.

Meta-cells were next labeled by taking each meta-cell and aggregating the transcriptome of similar cells to compute a pseudo-bulk. Gene signatures curated using Spectra and published HSC signatures were obtained and meta-cells were scored using the average expression of these gene signatures. Using these gene signatures as features, hierarchical clustering was then used and 5 meta-cell clusters reflecting lineage bias and age were identified. Each pseudo-bulk was labeled based on their cluster identity, but the independent pseudo-bulks were also retained for unbiased seq2PRINT analysis.

Previous studies reported the existence of two major HSC subtypes: (i) one with low lineage output, more megakaryocyte-biased, and expressing higher levels of quiescence and self-renewal markers (i.e., low-output or Mk-biased subtype), and (ii) one with higher multilineage output (i.e., multilineage subtype). The mouse aging HSC dataset was scored with gene signatures obtained from these studies and the existence of similar HSC subtypes was confirmed. A general increase in megakaryocyte bias during aging was also observed.

### AlphaFold3 Prediction of Runx1/Ets1 Interactions

AlphaFold3 predictions were generated for the Ets dimer predictions. The predicted structure and crystal structure were aligned in Pymol and visualized in Mol Viewer, which showed high overlap (RMSD=0.787 Å).

AlphaFold3 predictions were also generated for all de novo identified composite motifs with Runx and Ets predictions. Motif 10 matched the Runx1/Ets1 binding configuration resolved in PDB 4L0Z. An overlap of the predicted structure and the crystal structure for de novo motif #10 with the Runx1/Ets1 binding configuration was also determined. The alignment was performed in Pymol and visualized in Mol Viewer (RMSD=0.825 Å).

### CONCLUSION

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

1. A method for predicting DNA-protein interactions, comprising:
receiving chromatin accessibility data generated for at least a portion of a genome;
generating multi-scale footprint scores for window sizes ranging from 4 base pairs to 200 base pairs based on the chromatin accessibility data, the multi-scale footprint scores indicating protein binding to positions of the portion of the genome at different protein size scales;
training a deep learning model to generate predicted multi-scale footprint scores for a DNA sequence of interest using the multi-scale footprint scores as ground truth outputs and DNA sequences corresponding to the portion of the genome as inputs to the deep learning model during training; and
predicting the DNA-protein interactions for the DNA sequence of interest using the trained deep learning model, the predicting including:
receiving the DNA sequence of interest;
generating, by the trained deep learning model, the predicted multi-scale footprint scores for the DNA sequence of interest;
generating, by an attribution algorithm, sequence attribution scores for the DNA sequence of interest, the sequence attribution scores quantifying contributions of different DNA sequence features of the DNA sequence of interest to the predicted multi-scale footprint scores; and
predicting, by a transcription factor binding prediction model, transcription factor binding sites of the DNA sequence of interest for a plurality of different transcription factors based on the sequence attribution scores.

2. The method of claim 1, wherein the chromatin accessibility data comprises an assay for transposase-accessible chromatin using sequencing (ATAC-seq) data, and wherein generating the multi-scale footprint scores comprises combining a plurality of footprint scores generated for each base pair position of the portion of the genome, the plurality of footprint scores generated for the window sizes ranging from 4 base pairs to 200 base pairs by:
defining a window size for each footprint score of the plurality of footprint scores;
defining, for each footprint score, a center footprint region having a length equal to the window size and centered on a given base pair position of the portion of the genome;
defining, for each footprint score, two flanking regions each having a flanking region length equal to the window size, wherein a first of the two flanking regions is positioned left of the center footprint region and a second of the two flanking regions is positioned right of the center footprint region;
calculating, for each footprint score, a ratio of transposase insertions in the center footprint region to total insertions in both the center footprint region and the two flanking regions based on the ATAC-seq data corresponding to the center footprint region and the two flanking regions; and
determining a value for each footprint score by comparing the calculated ratio to a background distribution defining an expected distribution of transposon insertion ratios when no protein is bound at the given base pair position.

3. The method of claim 1, wherein the multi-scale footprint scores are represented as a matrix of elements at base-pair resolution, the matrix having a size L representing a sequence length of the portion of the genome times a number of scales in the multi-scale footprint scores.

4. The method of claim 1, wherein training the deep learning model comprises:
encoding the DNA sequences corresponding to the portion of the genome into one-hot encoded matrices for a plurality of training instances, wherein a given DNA sequence provides an input portion of a training instance of the plurality of training instances and corresponding multi-scale footprint scores generated from the chromatin accessibility data for the given DNA sequence provide an expected output portion of the training instance;
inputting the one-hot encoded matrices into the deep learning model;
generating, by the deep learning model for each training instance of the plurality of training instances, the predicted multi-scale footprint scores for a respective input one-hot encoded matrix based on parameters of the deep learning model; and
for each training instance of the plurality of training instances, adjusting the parameters of the deep learning model to minimize a difference between the predicted multi-scale footprint scores and the corresponding multi-scale footprint scores generated from the chromatin accessibility data.

5. The method of claim 4, wherein the deep learning model includes a convolutional neural network, and adjusting the parameters of the deep learning model includes adjusting weights and biases of the convolutional neural network.

6. The method of claim 1, further comprising:
identifying de novo motifs for the transcription factor binding sites based on the sequence attribution scores by clustering and aligning regions of the DNA sequence of interest having high sequence attribution scores.

7. The method of claim 1, wherein predicting, by the transcription factor binding prediction model, the transcription factor binding sites of the DNA sequence of interest for the plurality of different transcription factors based on the sequence attribution scores comprises:
inputting the sequence attribution scores into the transcription factor binding prediction model;
generating, by the transcription factor binding prediction model, transcription factor binding scores for regions of the DNA sequence of interest based on the sequence attribution scores corresponding to the regions, wherein the transcription factor binding scores include quantified likelihoods of transcription factor binding for the plurality of different transcription factors at the regions of the DNA sequence of interest;
identifying motif sites for a transcription factor of interest in the DNA sequence of interest via motif matching; and
indicating whether the transcription factor of interest is predicted to bind an identified motif site of the DNA sequence of interest based on a corresponding transcription factor binding score of the identified motif site.

8. The method of claim 1, further comprising:
receiving a training sample comprising chromatin immunoprecipitation followed by sequencing (ChIP-seq) data and associated DNA sequences, wherein the ChIP-seq data provide experimentally validated transcription factor binding sites for the associated DNA sequences;
generating, by the trained deep learning model in combination with the attribution algorithm, the sequence attribution scores for the associated DNA sequences of the training sample; and
training the transcription factor binding prediction model using the sequence attribution scores generated for the associated DNA sequences as inputs to the transcription factor binding prediction model and the ChIP-seq data as expected outputs of the transcription factor binding prediction model.

9. The method of claim 1, further comprising:
receiving single-cell ATAC-seq data generated for a plurality of cells having a plurality of different cell states;
aggregating the single-cell ATAC-seq data into pseudo-bulks each representing one of the plurality of different cell states; and
generating a low-rank adaptation (LoRA) model for predicting cell state-specific multi-scale footprint scores for a specific cell state of the plurality of different cell states by:
fine-tuning a subset of parameters of the trained deep learning model using a corresponding pseudo-bulk of the single-cell ATAC-seq data, the corresponding pseudo-bulk corresponding to the specific cell state; and
representing the fine-tuned subset of parameters as a low-rank decomposition.

10. The method of claim 9, further comprising:
generating multiple LoRA models for the plurality of different cell states represented in the pseudo-bulks, wherein each of the multiple LoRA models corresponds to a different one of the plurality of different cell states;
generating the cell state-specific predicted multi-scale footprint scores for the plurality of different cell states using the multiple LoRA models; and
predicting, by the transcription factor binding prediction model, cell state-specific transcription factor binding for each of the plurality of different cell states based on respective cell state-specific predicted multi-scale footprint scores.

11. A system for predicting DNA-protein interactions, comprising:
a processor;
a non-transitory computer-readable storage medium; and
a DNA-protein interaction analysis module implemented as instructions stored in the non-transitory computer-readable storage medium that, when executed by the processor, cause the processor to perform operations comprising:
receiving a DNA sequence of interest;
generating, using a deep learning model that has been trained with multi-scale footprint data for window sizes ranging from 4 base pairs to 200 base pairs, predicted multi-scale footprint scores for the DNA sequence of interest, wherein the predicted multi-scale footprint scores indicate predicted protein binding to positions of the DNA sequence of interest at different protein size scales;
generating, by an attribution algorithm, sequence attribution scores for the DNA sequence of interest based on the predicted multi-scale footprint scores, the sequence attribution scores including numerical values quantifying contributions of different DNA sequence features of the DNA sequence of interest to the predicted multi-scale footprint scores;
predicting, by a transcription factor binding prediction model, transcription factor binding sites of the DNA sequence of interest for a plurality of different transcription factors based on the sequence attribution scores; and
predicting, by a de novo motif discovery algorithm, de novo motifs for the transcription factor binding sites based on the sequence attribution scores.

12. The system of claim 11, wherein the multi-scale footprint data comprise a plurality of footprint scores for the window sizes ranging from 4 base pairs to 200 base pairs, and the operations further comprise:
generating, via a footprinting model of the DNA-protein interaction analysis module, the plurality of footprint scores by:
receiving an assay for transposase-accessible chromatin using sequencing (ATAC-seq) data generated for at least a portion of a genome;
defining, for each footprint score of the plurality of footprint scores, a window size for each footprint score of the plurality of footprint scores;
defining, for each footprint score of the plurality of footprint scores, a center footprint region having a length equal to the window size and centered on a given base pair position of the portion of the genome and two flanking regions each having a flanking region length equal to the window size, wherein a first of the two flanking regions is positioned left of the center footprint region and a second of the two flanking regions is positioned right of the center footprint region;
calculating, for each footprint score of the plurality of footprint scores, a ratio of transposase insertions in the center footprint region to total insertions in both the center footprint region and the two flanking regions based on the ATAC-seq data corresponding to the center footprint region and the two flanking regions; and
outputting a value for each footprint score of the plurality of footprint scores by comparing the calculated ratio to a background distribution defining an expected distribution of transposon insertion ratios when no protein is bound at the given base pair position of the portion of the genome.

13. The system of claim 11, wherein the operations further comprise:
training the deep learning model by:
receiving training data comprising a plurality of training instances, each training instance of the plurality of training instances including a DNA sequence as an input portion and an associated multi-scale footprint of the multi-scale footprint data as an expected output portion, wherein the associated multi-scale footprint is a multi-scale footprint generated by a footprinting model of the DNA-protein interaction analysis module from chromatin accessibility data obtained for the DNA sequence; and
for each training instance of the plurality of training instances:
encoding the DNA sequence into a one-hot encoded matrix;
generating, by the deep learning model, a predicted multi-scale footprint based on the one-hot encoded matrix; and
adjusting parameters of the deep learning model based on the predicted multi-scale footprint compared to the associated multi-scale footprint of the multi-scale footprint data.

14. The system of claim 11, wherein predicting the de novo motifs comprises:
clustering, via a de novo motif discovery algorithm of the DNA-protein interaction analysis module, regions of the DNA sequence of interest having high sequence attribution scores;
aligning, via the de novo motif discovery algorithm, the clustered regions; and
outputting, by the de novo motif discovery algorithm, recurring sequence patterns corresponding to previously uncharacterized DNA binding sites for proteins based on the aligned clustered regions.

15. The system of claim 11, wherein the operations further comprise:
receiving single-cell ATAC-seq data generated for a plurality of cells having a plurality of different cell states;
aggregating the single-cell ATAC-seq data into pseudo-bulks each representing one of the plurality of different cell states;
generating, from the deep learning model, a low-rank adaptation (LoRA) model for predicting cell state-specific multi-scale footprint scores for a specific cell state of the plurality of different cells states by:
fine-tuning a subset of parameters of the trained deep learning model using a corresponding pseudo-bulk of the single-cell ATAC-seq data, the corresponding pseudo-bulk corresponding to the specific cell state; and
representing the fine-tuned subset of parameters as a low-rank decomposition; and
predicting DNA-protein interactions for the DNA sequence of interest using the LoRA model.

16. A method for analyzing DNA-protein interactions in single-cell data, comprising:
receiving bulk chromatin accessibility data for at least a portion of a genome, the bulk chromatin data obtained from cells having a plurality of different cell states;
generating multi-scale footprint scores for window sizes ranging from 4 base pairs to 200 base pairs based on the bulk chromatin accessibility data, the multi-scale footprint scores indicating protein binding to positions of the portion of the genome at different protein size scales;
training a deep learning model to generate predicted multi-scale footprint scores for a DNA sequence of interest using the multi-scale footprint scores as ground truth outputs and DNA sequences corresponding to the portion of the genome as inputs to the deep learning model during training;
receiving single-cell chromatin accessibility data;
aggregating the single-cell chromatin accessibility data into pseudo-bulks each representing one of the plurality of different cell states;
generating a plurality of low-rank adaptation (LoRA) models for predicting cell state-specific multi-scale footprint scores for the DNA sequence of interest, each LoRA model of the plurality of LoRA models corresponding to a specific cell state of the plurality of different cell states, by fine-tuning a subset of parameters of the trained deep learning model using a corresponding pseudo-bulk of the single-cell chromatin accessibility data, the corresponding pseudo-bulk corresponding to the specific cell state for a given LoRA model of the plurality of LoRA models; and
predicting the cell state-specific DNA-protein interactions for the DNA sequence of interest via the plurality of LoRA models.

17. The method of claim 16, wherein the fine-tuned subset of parameters are represented as a low-rank decomposition.

18. The method of claim 16, further comprising:
training the deep learning model by:
encoding DNA sequences corresponding to the bulk chromatin accessibility data into one-hot encoded matrices for a plurality of training instances, wherein a given DNA sequence provides an input portion of a training instance of the plurality of training instances and corresponding multi-scale footprint scores generated from the bulk chromatin accessibility data for the given DNA sequence provide an expected output portion of the training instance;
inputting the one-hot encoded matrices into the deep learning model;
generating, by the deep learning model for each training instance of the plurality of training instances, the predicted multi-scale footprint scores for a respective input one-hot encoded matrix based on parameters of the deep learning model; and
for each training instance of the plurality of training instances, adjusting the parameters of the deep learning model to minimize a difference between the predicted multi-scale footprint scores and the corresponding multi-scale footprint score generated from the bulk chromatin accessibility data.

19. The method of claim 16, wherein predicting the cell state-specific DNA-protein interactions for the DNA sequence of interest via the plurality of LoRA models comprises:
generating, by a LoRA model of the plurality of LoRA models corresponding to a cell state of interest, predicted cell state-specific multi-scale footprint scores for the DNA sequence of interest;
generating, by an attribution algorithm, sequence attribution scores for the DNA sequence of interest based on the predicted cell state-specific multi-scale footprint scores, the sequence attribution scores including numerical values quantifying contributions of different DNA sequence features of the DNA sequence of interest to the predicted cell state-specific multi-scale footprint scores; and
predicting, by a transcription factor binding prediction model, transcription factor binding sites of the DNA sequence of interest based on the sequence attribution scores.

20. The method of claim 16, further comprising:
identifying cell state-specific regulatory elements by comparing the cell state-specific DNA-protein interactions predicted using different LoRA models of the plurality of LoRA models; and
tracking changes in the cell state-specific regulatory elements across a cellular differentiation trajectory.
